(19) United States
(12) Patent Application Publication
(10) Pub. No.: US 2022/0395611 A1
(43) Pub. Date: Dec. 15, 2022

(54) BIOMATERIALS FOR BONE TISSUE ENGINEERING

(71) Applicants: Spiderwort Inc., Ottawa (CA); University of Ottawa, Ottawa (CA)

(72) Inventors: Maxime LEBLANC LATOUR, Gatineau (CA); Ryan HICKEY, Ottawa (CA); Charles M. CUERRIER, Ottawa (CA); Andrew E. PELLING, Ottawa (CA); Maryam TARAR, Ottawa (CA); Isabelle CATELAS, Ottawa (CA)

(21) Appl. No.: 17/774,875

(22) PCT Filed: Dec. 18, 2020
(86) PCT No.: PCT/CA2020/051750

§ 371 Date: May 6, 2022

Related U.S. Application Data
(60) Provisional application No. 62/950,544, filed on Dec. 19, 2019.

(51) Int. Cl. A61L 27/36; A61L 27/52; A61L 27/24; A61L 27/20; A61L 27/38; A61L 27/12; A61L 27/04; A61L 27/10

(52) U.S. Cl. CPC A61L 27/3637; A61L 27/52; A61L 27/24; A61L 27/20; A61L 27/3821; A61L 27/12; A61L 27/047; A61L 27/10; A61L 27/3691; A61L 2430/02; A61L 2430/12; A61L 2400/12; A61L 2430/40

(57) ABSTRACT

Provided herein are scaffold biomaterials including a decellularized plant or fungal tissue from which cellular materials and nucleic acids of the tissue are removed, the decellularized plant or fungal tissue having a 3-dimensional porous structure; wherein the decellularized plant or fungal tissue may optionally be at least partially coated or mineralized, wherein the scaffold biomaterial may optionally further include a protein-based hydrogel and/or a polysaccharide-based hydrogel, or both. Also provided herein are methods and uses of such scaffold biomaterials, including methods of manufacture as well as methods and uses for bone tissue engineering, for example.

### CROSS-REFERENCE TO RELATED APPLICATIONS

The present application claims priority to U.S. provisional patent application No. 62/950,544, entitled “Biomaterials for Bone Tissue Engineering”, filed on Dec. 19, 2019, the contents of which are incorporated herein by reference in their entirety.

### FIELD OF INVENTION

The present invention relates generally to scaffold biomaterials. More specifically, the present invention relates to scaffold biomaterials comprising decellularized plant or fungal tissue, for use in bone tissue engineering.

### BACKGROUND

Large bone defects caused by injury or disease often require biomaterial grafts to completely regenerate [1]. Typically, techniques designed to enhance bone tissue regeneration have commonly employed autologous, allogeneic, xenogeneic, or synthetic grafts [2]. Autologous bone grafting, in which the material is derived from the patient, is considered the “gold standard” grafting practice in large bone defect repair, but there are several drawbacks including size and shape limitations, tissue availability, and donor site morbidity [3]. Autologous grafting procedures are prone to infections, subsequent fractures, hematoma formation at the donor or repaired site, and post-operative pain [4]. Bone tissue engineering provides a potential alternative to traditional bone grafting methods [5].

Bone tissue engineering (BTE) combines the use of structural biomaterials and cells to create new functional bone tissue. The biomaterials used for BTE typically aim to provide similar mechanical properties and architecture to the native bone matrix [6]. Previous studies have shown that the optimal pore size for biomaterials used for BTE is approximately 100-200 μm [7], and elastic modulus is 0.1 to 20 GPa depending on the grafting site [8]. Moreover, the porosity and pore interconnectivity are two important factors that may affect cell migration, nutrient diffusion, and angiogenesis [8]. BTE has shown promising results with a diverse set of biomaterials developed as an alternative to bone grafts. These biomaterials include osteoinductive materials, hybrid materials, and advanced hydrogels [8]. Osteoinductive materials induce the surrounding environment to form de novo bone structure. Hybrid materials are made of synthetic and/or natural polymers [8]. Advanced hydrogels mimic the ECM and deliver the required bioactive agents to promote bone tissue integration [8]. Hydroxyapatite, a calcium apatite, is a material which may be used for BTE due to its biocompatibility, composition, and its role in the mineral structure in native bones [9]. Another type of biomaterial for BTE is bioactive glass, which stimulates specific cell responses to activate genes for osteogenesis [10], [11]. Biodegradable polymers such as poly (glycolic acid) and Poly (lactic acid) are also used for BTE [12]. Natural (or naturally derived) polymers such as chitosan, chitin and bacterial cellulose have been tested for BTE as well [13]. Although these polymers, either natural or synthetic, may show some potential in BTE, extensive, difficult, and/or costly protocols are employed to obtain a functional biomaterial and/or macrostructure, and each have respective limitations.

Alternative, additional, and/or improved biomaterials for bone tissue engineering (BTE) and/or methods for the preparation thereof are desirable.

### SUMMARY OF INVENTION

Provided herein are materials (biomaterials) that may be used in bone tissue engineering applications, such as in the repair and/or regeneration of damaged, degraded, defective, and/or missing bone structures. The present inventors have now developed scaffold biomaterials comprising decellularized plant or fungal tissue, wherein the decellularized plant or fungal tissue may optionally be at least partially coated or mineralized, wherein the scaffold biomaterial may optionally further include a protein-based hydrogel and/or a polysaccharide-based hydrogel, or both. Experimental studies described herein indicate that such scaffold biomaterials may be biocompatible, and may support growth of pre-osteoblasts, which may be differentiated in the scaffold biomaterials. Accordingly, scaffold biomaterials as described herein may be used for bone tissue engineering, such as in the repair and/or regeneration of damaged, degraded, defective, and/or missing bone structures, for example. Results indicate that protein-based hydrogels, such as collagen hydrogels, may be used in such scaffold biomaterials, and that pre-mineralization of scaffold biomaterials with, for example, hydroxyapatite may be used.

In an embodiment, there is provided herein a scaffold biomaterial comprising:
a decellularized plant or fungal tissue from which cellular materials and nucleic acids of the tissue are removed, the decellularized plant or fungal tissue comprising a 3-dimensional porous structure; and
a protein-based hydrogel, a polysaccharide-based hydrogel, or a combination thereof.

In another embodiment of the above scaffold biomaterial, the protein-based hydrogel may comprise collagen, osteonectin, osteopontin, bone sialoprotein, osteocalcin, fibronectin, laminin, a proteoglycan, bone morphogenetic protein, other matrix protein(s) or any combinations thereof.

In another embodiment of any of the above scaffold biomaterials, the polysaccharide-based hydrogel may comprise agarose, alginate, hyaluronic acid, or another carbohydrate-based hydrogel.

In certain embodiments, the decellularized plant or fungal tissue and/or the protein-based hydrogel and/or polysaccharide-based hydrogel may comprise one or more markers of osteogenic differentiation, such as osteonectin, osteopontin, bone sialoprotein, osteocalcin, fibronectin, laminin, a proteoglycan, or any combinations thereof. In certain embodiments, the decellularized plant or fungal tissue and/or the protein-based hydrogel and/or the polysaccharide-based hydrogel may comprise one or more proteins found in normal bone matrix.

In still another embodiment of any of the above scaffold biomaterial or scaffold biomaterials, the protein-based hydrogel may comprise a collagen hydrogel.

In yet another embodiment of any of the above scaffold biomaterial or scaffold biomaterials, the protein-based hydrogel may comprise collagen I.

In another embodiment of any of the above scaffold biomaterial or scaffold biomaterials, the decellularized plant or fungal tissue may comprise a pore size of about 100 to about 200 μm, or of about 150 to about 200 μm.

In still another embodiment of any of the above scaffold biomaterial or scaffold biomaterials, the decellularized plant or fungal tissue may comprise decellularized apple hypanthium tissue.

In another embodiment of any of the above scaffold biomaterial or scaffold biomaterials, the scaffold biomaterial may comprise one or more bone-relevant cell types such as preosteoblasts, osteoblasts, osteoclasts, and/or mesenchymal stem cells, or any combinations thereof. In another embodiment, the scaffold biomaterial may be pre-seeded with one or more bone-relevant cell types such as preosteoblasts, osteoblasts, osteoclasts, and/or mesenchymal stem cells, or any combination thereof.

In still another embodiment of any of the above scaffold biomaterial or scaffold biomaterials, the scaffold biomaterial may have a Young's moduli between about 20 kPa to about 1 MPa.

In still another embodiment of any of the above scaffold biomaterial or scaffold biomaterials, pore walls of the decellularized plant or fungal tissue may be mineralized by the osteoblasts.

In yet another embodiment of any of the above scaffold biomaterial or scaffold biomaterials, the decellularized plant or fungal tissue may be at least partially coated or mineralized.

In another embodiment of any of the above scaffold biomaterial or scaffold biomaterials, the decellularized plant or fungal tissue may be at least partially coated or mineralized with apatite, osteocalcium phosphate, a biocompatible ceramic, a biocompatible glass, a biocompatible metal nanoparticle, nanocrystalline cellulose, or any combinations thereof.

In yet another embodiment of any of the above scaffold biomaterial or scaffold biomaterials, the decellularized plant or fungal tissue may be at least partially coated or mineralized with apatite.

In still another embodiment of any of the above scaffold biomaterial or scaffold biomaterials, the apatite may comprise hydroxyapatite.

In another embodiment, there is provided herein a scaffold biomaterial comprising:
a decellularized plant or fungal tissue from which cellular materials and nucleic acids of the tissue are removed, the decellularized plant or fungal tissue comprising a 3-dimensional porous structure;
the decellularized plant or fungal tissue being at least partially coated or mineralized.

In another embodiment of the above scaffold biomaterial, the decellularized plant or fungal tissue may be at least partially coated or mineralized with apatite, osteocalcium phosphate, a biocompatible ceramic, a biocompatible glass, a biocompatible metal nanoparticle, nanocrystalline cellulose, or any combinations thereof.

In still another embodiment of any of the above scaffold biomaterial or scaffold biomaterials, the decellularized plant or fungal tissue may be at least partially coated or mineralized with apatite.

In yet another embodiment of any of the above scaffold biomaterial or scaffold biomaterials, the apatite may comprise hydroxyapatite.

In another embodiment of any of the above scaffold biomaterial or scaffold biomaterials, the decellularized plant or fungal tissue may comprise apple.

In still another embodiment of any of the above scaffold biomaterial or scaffold biomaterials, the decellularized plant or fungal tissue may be at least partially coated or mineralized with apatite by alternating exposure to solutions of calcium chloride and disodium phosphate.

In yet another embodiment of any of the above scaffold biomaterial or scaffold biomaterials, the scaffold biomaterial may further comprise a protein-based hydrogel or a polysaccharide-based hydrogel or both.

In still another embodiment of any of the above scaffold biomaterial or scaffold biomaterials, the protein-based hydrogel may comprise collagen, osteonectin, osteopontin, bone sialoprotein, osteocalcin, fibronectin, laminin, a proteoglycan, bone morphogenetic protein, other matrix protein(s), or any combinations thereof.

In another embodiment of any of the above scaffold biomaterial or scaffold biomaterials, the polysaccharide-based hydrogel may comprise agarose, alginate, hyaluronic acid, or another carbohydrate-based hydrogel.

In certain embodiments, the decellularized plant or fungal tissue and/or the protein-based hydrogel and/or the polysaccharide-based hydrogel may comprise one or more markers of osteogenic differentiation, such as osteonectin, osteopontin, bone sialoprotein, osteocalcin, fibronectin, laminin, a proteoglycan, or any combinations thereof. In certain embodiments, the decellularized plant or fungal tissue and/or the protein-based hydrogel and/or the polysaccharide-based hydrogel may comprise one or more proteins found in normal bone matrix.

In yet another embodiment of any of the above scaffold biomaterial or scaffold biomaterials, the protein-based hydrogel may comprise a collagen hydrogel.

In another embodiment of any of the above scaffold biomaterial or scaffold biomaterials, the protein-based hydrogel may comprise collagen I.

In still another embodiment of any of the above scaffold biomaterial or scaffold biomaterials, the decellularized plant or fungal tissue may be cellulose-based, chitin-based, chitosan-based, lignin-based, hemicellulose-based, or pectin-based, or any combination thereof.

In another embodiment of any of the above scaffold biomaterial or scaffold biomaterials, the plant or fungal tissue may comprise a tissue from apple hypanthium (Malus pumila) tissue, a fern (Monilophytes) tissue, a turnip (Brassica rapa) root tissue, a gingko branch tissue, a horsetail (equisetum) tissue, a hermocallis hybrid leaf tissue, a kale (Brassica oleracea) stem tissue, a conifers Douglas Fir (Pseudotsuga menziesii) tissue, a cactus fruit (pitaya) flesh tissue, a Maculata Vinca tissue, an Aquatic Lotus (Nelumbo nucifera) tissue, a Tulip (Tulipa gesneriana) petal tissue, a Plantain (Musa paradisiaca) tissue, a broccoli (Brassica oleracea) stem tissue, a maple leaf (Acer psuedoplatanus) stem tissue, a beet (Beta vulgaris) primary root tissue, a green onion (Allium cepa) tissue, a orchid (Orchidaceae) tissue, turnip (Brassica rapa) stem tissue, a leek (Allium ampeloprasum) tissue, a maple (Acer) tree branch tissue, a celery (Apium graveolens) tissue, a green onion (Allium cepa) stem tissue, a pine tissue, an aloe vera tissue, a watermelon (Citrullus lanatus var. lanatus) tissue, a Creeping Jenny (Lysimachia nummularia) tissue, a cactae tissue, a Lychnis Alpina tissue, a rhubarb (Rheum rhabarbarum) tissue, a pumpkin flesh (Cucurbita pepo) tissue, a Dracena (Asparagaceae) stem tissue, a Spiderwort (Tradescantia virginiana) stem tissue, an Asparagus (Asparagus officinalis) stem tissue, a mushroom (Fungi) tissue, a fennel (Foeniculum vulgare) tissue, a rose (Rosa) tissue, a carrot (Daucus carota) tissue, or a pear (Pomaceous) tissue, or a genetically altered tissue produced via direct genome modification or through selective breeding, or any combinations thereof.

In still another embodiment of any of the above scaffold biomaterial or scaffold biomaterials, the scaffold biomaterial may further comprise living cells, in particular non-native cells, on and/or within the decellularized plant or fungal tissue.

In still another embodiment of any of the above scaffold biomaterial or scaffold biomaterials, the living cells may be animal cells.

In still another embodiment of any of the above scaffold biomaterial or scaffold biomaterials, the living cells may be mammalian cells.

In still another embodiment of any of the above scaffold biomaterial or scaffold biomaterials, the living cells may be human cells.

In still another embodiment of any of the above scaffold biomaterial or scaffold biomaterials, the scaffold biomaterial may comprise two or more subunits which are glued, cross-linked, or interlocked together.

In another embodiment of any of the above scaffold biomaterial or scaffold biomaterials, the decellularized plant or fungal tissue may comprise two or more different decellularized plant or fungal tissues derived from different tissues or different sources.

In yet another embodiment of any of the above scaffold biomaterial or scaffold biomaterials, the two or more different decellularized plant or fungal tissues may be glued, cross-linked, or interlocked together.

In another embodiment of any of the above scaffold biomaterial or scaffold biomaterials, the scaffold biomaterial may be for use in bone tissue engineering (BTE).

In another embodiment, there is provided herein a bone graft comprising any of the scaffold biomaterial or biomaterials as described herein.

In another embodiment, there is provided herein a use of any of the scaffold biomaterial or scaffold biomaterials as described herein for bone tissue engineering (BTE), for bone grafting, for repair or regeneration of bone, or any combination thereof.

In another embodiment, there is provided herein a use of any of the scaffold biomaterial or scaffold biomaterials as described herein for any one or more of: craniofacial reconstructive surgery; dental and/or maxillofacial reconstructive surgery; major bone defect and/or trauma reconstruction; bone filler applications; implant stabilization; and/or drug delivery; or any combinations thereof.

In another embodiment, there is provided herein a use of any of the scaffold biomaterial or scaffold biomaterials as described herein in a dental bone filler application.

In another embodiment, there is provided herein a use of any of the scaffold biomaterial or scaffold biomaterials as described herein as stress shielding reducers for large implants.

In yet another embodiment, there is provided herein a use of any of the scaffold biomaterial or scaffold biomaterials as described herein for promoting active osteogenesis; for implanting to repair critical and/or non-critical size defects; to provide mechanical support during bone repair; to substitute in loss or injury of long bones, calvarial bones, maxillofacial bones, dental, and/or jaw bones; for orthodontal and/or peri dental grafts, such as alveolar ridge augmentation, tooth loss, tooth implants and/or reconstructive surgery; for grafting at specific site(s) to augment bone volume due to loss from osteoporosis, bone loss due to age, previous implant, and/or injuries; or to improve bone-implant tissue integration; or any combinations thereof.

In another embodiment, there is provided herein a method for engineering bone tissue; for bone grafting; for repair or regeneration of bone; for craniofacial reconstructive surgery; for dental and/or maxillofacial reconstructive surgery; for major bone defect and/or trauma reconstruction; for dental or other bone filler application; for implant stabilization; for stress shielding of a large implant; for promoting active osteogenesis; for repairing critical and/or non-critical size defects; for provide mechanical support during bone repair; for substituting for loss or injury of long bones, calvarial bones, maxillofacial bones, dental, and/or jaw bones; for orthodontal and/or peri dental grafting such as alveolar ridge augmentation, tooth loss, tooth implants and/or reconstructive surgery; for grafting at a specific site to augment bone volume due to loss from osteoporosis, bone loss due to age, previous implant, and/or injuries; for improving bone-implant tissue integration; or for drug delivery; or for any combinations thereof; said method comprising:
providing any of the scaffold biomaterial or scaffold biomaterials as described herein; and
implanting the scaffold biomaterial into a subject in need thereof at a site or region in need thereof.

In another embodiment, there is provided herein a method for producing a scaffold biomaterial, said method comprising:
providing a decellularized plant or fungal tissue from which cellular materials and nucleic acids of the tissue are removed, the decellularized plant or fungal tissue comprising a 3-dimensional porous structure; and
introducing a protein-based hydrogel, a polysaccharide-based hydrogel, or both, into the decellularized plant or fungal tissue.

In another embodiment of the above method, the protein-based hydrogel may comprise collagen, osteonectin, osteopontin, bone sialoprotein, osteocalcin, fibronectin, laminin, a proteoglycan, bone morphogenetic protein, other matrix protein(s), or any combinations thereof.

In another embodiment of any of the above method or methods, the polysaccharide-based hydrogel may comprise agarose, alginate, hyaluronic acid, or another carbohydrate-based hydrogel.

In certain embodiments, the decellularized plant or fungal tissue and/or the protein-based hydrogel and/or the polysaccharide-based hydrogel may comprise one or more markers of osteogenic differentiation, such as osteonectin, osteopontin, bone sialoprotein, osteocalcin, fibronectin, laminin, a proteoglycan, or any combinations thereof. In certain embodiments, the decellularized plant or fungal tissue and/or the protein-based hydrogel and/or the polysaccharide-based hydrogel may comprise one or more proteins found in normal bone matrix.

In another embodiment of the above method or methods, the protein-based hydrogel may comprise a collagen hydrogel.

In still another embodiment of any of the above method or methods, the protein-based hydrogel may comprise collagen I.

In another embodiment, there is provided herein a method for producing a scaffold biomaterial, said method comprising:
providing a decellularized plant or fungal tissue from which cellular materials and nucleic acids of the tissue are removed, the decellularized plant or fungal tissue comprising a 3-dimensional porous structure; and
at least partially coating or mineralizing the decellularized plant or fungal tissue.

In another embodiment of the above method, the decellularized plant or fungal tissue may be at least partially coated or mineralized with apatite, osteocalcium phosphate, a biocompatible ceramic, a biocompatible glass, a biocompatible metal nanoparticle, nanocrystalline cellulose, or any combinations thereof.

In still another embodiment of any of the above method or methods, the decellularized plant or fungal tissue may be at least partially coated or mineralized with apatite.

In yet another embodiment of any of the above method or methods, the apatite may comprise hydroxyapatite.

In another embodiment of any of the above method or methods, the step of coating or mineralizing the decellularized plant or fungal tissue may comprise subjecting the decellularized plant or fungal tissue to alternating exposures to solutions of calcium chloride and disodium phosphate.

In still another embodiment of any of the above method or methods, the method may further comprise introducing a protein-based hydrogel and/or a polysaccharide-based hydrogel to the scaffold biomaterial.

In another embodiment of any of the above method or methods, the protein-based hydrogel may comprise collagen, osteonectin, osteopontin, bone sialoprotein, osteocalcin, fibronectin, laminin, a proteoglycan, bone morphogenetic protein, other matrix protein(s), or any combinations thereof.

In another embodiment of any of the above method or methods, the polysaccharide-based hydrogel may comprise agarose, alginate, hyaluronic acid, or another carbohydrate-based hydrogel.

In certain embodiments, the decellularized plant or fungal tissue and/or the protein-based hydrogel and/or the polysaccharide-based hydrogel may comprise one or more markers of osteogenic differentiation, such as osteonectin, osteopontin, bone sialoprotein, osteocalcin, fibronectin, laminin, a proteoglycan, or any combinations thereof. In certain embodiments, the decellularized plant or fungal tissue and/or the protein-based hydrogel and/or the polysaccharide-based hydrogel may comprise one or more proteins found in normal bone matrix.

In yet another embodiment of any of the above method or methods, the protein-based hydrogel may comprise a collagen hydrogel.

In still another embodiment of any of the above method or methods, the protein-based hydrogel may comprise collagen I.

In yet another embodiment of any of the above method or methods, the method may further comprise a step of introducing living cells, in particular non-native cells, on and/or within the decellularized plant or fungal tissue.

In another embodiment of any of the above method or methods, the living cells may be animal cells.

In yet another embodiment of any of the above method or methods, the living cells may be mammalian cells.

In still another embodiment of any of the above method or methods, the living cells may be human cells.

In another embodiment of any of the above method or methods, the cells may be one or more bone-relevant cell types such as preosteoblasts, osteoblasts, osteoclasts, and/or mesenchymal cells, or any combinations thereof. In another embodiment, the method may comprise a step of pre-seeding with one or more bone-relevant cell types such as preosteoblasts, osteoblasts, osteoclasts, and/or mesenchymal stem cells, or any combinations thereof.

In another embodiment, there is provided herein a kit comprising any one or more of:
a decellularized plant or fungal tissue from which cellular materials and nucleic acids of the tissue are removed, the decellularized plant or fungal tissue comprising a 3-dimensional porous structure;
a protein-based hydrogel;
a polysaccharide-based hydrogel;
apatite;
calcium chloride;
disodium phosphate;
osteocalcium phosphate;
a biocompatible ceramic;
a biocompatible glass;
a biocompatible metal nanoparticle;
nanocrystalline cellulose;
mammalian cells, such as one or more bone-relevant cell types such as preosteoblasts, osteoblasts, osteoclasts, and/or mesenchymal stem cells, or any combinations thereof (in certain embodiments, the decellularized plant or fungal tissue and/or the protein-based hydrogel and/or the polysaccharide-based hydrogel may be pre-seeded with one or more of such mammalian cells and/or bone-relevant cell types such as preosteoblasts, osteoblasts, osteoclasts, and/or mesenchymal stem cells, or any combinations thereof);
plant or fungal tissue, decellularization reagents, or both;
a buffer; and/or
instructions for performing any of the method or methods as described herein.

In another embodiment of the above kit, the protein-based hydrogel may comprise collagen, osteonectin, osteopontin, bone sialoprotein, osteocalcin, fibronectin, laminin, a proteoglycan, bone morphogenetic protein, other matrix protein(s), or any combinations thereof.

In another embodiment of any of the above kit or kits, the polysaccharide-based hydrogel may comprise agarose, alginate, hyaluronic acid, or another carbohydrate-based hydrogel.

In certain embodiments, the decellularized plant or fungal tissue and/or the protein-based hydrogel and/or the polysaccharide-based hydrogel may comprise one or more markers of osteogenic differentiation, such as osteonectin, osteopontin, bone sialoprotein, osteocalcin, fibronectin, laminin, a proteoglycan, or any combinations thereof. In certain embodiments, the decellularized plant or fungal tissue and/or the protein-based hydrogel and/or the polysaccharide-based hydrogel may comprise one or more proteins found in normal bone matrix.

In another embodiment of any of the above kit or kits, the protein-based hydrogel may comprise a collagen hydrogel.

In still another embodiment of any of the above kit or kits, the protein-based hydrogel may comprise collagen I.

In still another embodiment of any of the above kit or kits, the apatite may comprise hydroxyapatite.

In another embodiment, there is provided herein a method for differentiating cartilage or bone precursor cells to become cartilage or bone tissue cells, said method comprising:
culturing the cartilage or bone precursor cells on any of the scaffold biomaterial or scaffold biomaterials as described herein in a differentiation media;
wherein the culturing includes exposing the cultured cells to an increased atmospheric pressure above ambient pressure at least once.

In another embodiment, there is provided herein a method for differentiating cartilage or bone precursor cells to become cartilage or bone tissue cells, said method comprising:
culturing the cartilage or bone precursor cells on any of the scaffold biomaterial or scaffold biomaterials as described herein in a differentiation media;
wherein the culturing includes at least one treatment period during which the cultured cells are exposed to an increased atmospheric pressure above ambient pressure for at least part of the treatment period, wherein the treatment period is at least about 10 minutes in duration and is performed at least once per week;

thereby differentiating the cartilage or bone precursor cells into cartilage or bone tissue cells.

In yet another embodiment of any of the above method or methods, the cultured cells may be returned to a low or ambient pressure condition after each exposure to the increased atmospheric pressure.

In yet another embodiment of any of the above method or methods, the treatment period may comprise alternating the cultured cells between a low or ambient pressure condition, and an increased atmospheric pressure condition.

In another embodiment of any of the above method or methods, the treatment period may comprise oscillating atmospheric pressure to which the cells are exposed between a low or ambient pressure and an increased atmospheric pressure.

In yet another embodiment of any of the above method or methods, the treatment period may comprise oscillating atmospheric pressure to which the cells are exposed between a low or ambient pressure and an increased atmospheric pressure at a frequency of about 1-10 Hz.

In yet another embodiment of any of the above method or methods, the treatment period may comprise oscillating atmospheric pressure to which the cells are exposed between a low or ambient pressure and an increased atmospheric pressure, wherein the low or ambient pressure is ambient pressure (i.e. typically about 101 kPa+about 0 kPa) and the increased atmospheric pressure is about +280 kPa above ambient pressure (i.e. typically about 101 kPa+about 280 kPa=about 381 kPa), and optionally wherein the oscillating is at a frequency of about 1-10 Hz.

In still another embodiment of any of the above method or methods, the treatment period may comprise exposing the cultured cells to increased atmospheric pressure for a sustained duration.

In yet another embodiment of any of the above method or methods, the treatment period may comprise exposing the cultured cells to a substantially constant increased atmospheric pressure for a sustained duration.

In another embodiment of any of the above method or methods, the treatment period may be about 1 hour in duration, or longer.

In still another embodiment of any of the above method or methods, the treatment period may be performed once daily, or more than once daily.

In yet another embodiment of any of the above method or methods, the culturing may be performed for at least about 1 week.

In another embodiment of any of the above method or methods, the culturing may be performed for about 2 weeks, or longer.

In still another embodiment of any of the above method or methods, the increased atmospheric pressure may be applied as hydrostatic pressure.

In yet another embodiment of any of the above method or methods, the increased atmospheric pressure may be applied by modulating the pressure of a gas phase above the cultured cells.

In still another embodiment of any of the above method or methods, the increased atmospheric pressure may be about +280 kPa above ambient pressure (i.e. typically about 101 kPa+about 280 kPa=about 381 kPa).

### BRIEF DESCRIPTION OF DRAWINGS

These and other features will become further understood with regard to the following description and accompanying drawings, wherein:

FIG. 1 shows photographs of an apple-derived cellulose scaffold after removal of the plant cells and surfactant (A) (scale bar=2 mm—also applies to B and C), as well as a bare scaffold (B) and a calcified composite hydrogel scaffold (C) after 4-week in osteogenic differentiation medium. Representative confocal laser scanning microscope images showing seeded cells on a bare scaffold (D) (scale bar=50 μm—also applies to E) and a composite hydrogel scaffold (E). The scaffolds were stained for cellulose (red) and for cell nuclei (blue) using propidium iodide and DAPI staining respectively. Three different scaffolds were analyzed for each experimental condition. FIG. 1A shows an apple-derived cellulose scaffold after removal of the plant cells and surfactant; FIG. 1B shows a MC3T3-E1 seeded scaffold after 4-week in osteogenic differentiation medium, and FIG. 1D shows a representative confocal laser scanning microscope image showing seeded cells in a scaffold;

FIG. 2 shows pore size distribution of decellularized apple-derived cellulose scaffolds, before MC3T3 cell seeding, from maximum projections in the Z axis of confocal images. A total of 54 pores were analyzed in 3 different scaffolds (6 pores in 3 randomly selected areas per scaffold);

FIG. 3 shows Young's modulus of cell-seeded bare and composite hydrogel (with collagen) scaffolds after 4-weeks of culture in either non-differentiation or differentiation medium. Decellularized apple-derived cellulose scaffolds without cells served as a control. Statistical significance was determined using a one-way ANOVA and Tukey post-hoc tests. (N-D) and (D): scaffolds incubated in non-differentiation and differentiation medium, respectively. Data are presented as means±S.E.M. of three replicate samples per condition;

FIG. 4 shows photographs of scaffolds stained with 5-bromo-4-chloro-3′-indolyphosphate and nitro-blue tetrazolium (BCIP/NBT) (A-E) or Alizarin Red S (ARS) (F-J) (scale bar in A=2 mm—applies to all). The alkaline phosphatase (ALP) activity was visualized using BCIP/NBT staining. The control scaffolds (bare scaffolds without cells, “CTRL”) (A) did not stain with BCIP/NBT. Stronger ALP activity was visualized by stronger blue contrast in the bare scaffolds (D) and the composite hydrogel scaffolds (E) containing differentiated cells “D”, compared to their counterparts with non-differentiated cells “N-D” (B and C, respectively). For the ARS staining, the control scaffolds (bare scaffolds without cells) (F), the bare scaffolds with non-differentiated cells (G) and the composite hydrogel scaffolds with non-differentiated cells (H), displayed a light red color. The calcium deposition was highlighted with a strong, dark red color in the bare scaffolds (I) and the composite hydrogel scaffolds (J) containing differentiated cells. Three different scaffolds were analyzed for each experimental condition;

FIG. 5 shows representative images of scaffold histological cross-sections. Paraffin-embedded scaffolds were cut into 5 μm-thick sections and stained with Hematoxylin and Eosin (H&E) to visualize cell invasion (A, B, E and F) or Von Kossa (VK) to visualize mineralization (C, D, G and H) (scale bar in A=1 mm—applies to all). Bare scaffolds and composite hydrogel scaffolds were infiltrated with MC3T3-E1 cells with multiple nuclei and cytoplasm visible at the periphery and throughout the scaffolds (A, B, E and F, blue and pink, respectively). Collagen was also visible in pale pink and more pronounced in the composite hydrogel scaffolds. The pore walls in the bare scaffolds and in the composite hydrogel scaffolds only showed the presence of mineralization at the periphery of the scaffolds when cultured in non-differentiation medium (C, G). The pore walls in the bare scaffolds and in the composite hydrogel scaffolds were entirely stained in black when cultured in differentiation medium (D, H). The bare scaffolds cultured in non-differentiation medium were damaged upon sectioning (A, C). (N-D) and (D): scaffolds incubated in non-differentiation and differentiation medium, respectively. The analysis was performed on one scaffold of each type cultured in non-differentiation medium and on 2 scaffolds of each type cultured in differentiation medium;

FIG. 6 shows representative scanning electron microscopy micrographs (A-C) and energy-dispersive spectra (D-F): Bare scaffold (A) and composite hydrogel scaffold (B) with MC3T3-E1 cells after differentiation, along with non-seeded cellulose scaffold (C), were gold-coated and imaged using a JEOL JSM-7500F FESEM scanning electron microscope at 2.0 kV (scale bar in A=20 m—applies to all). Collagen fibres are visible (B inset, scale bar=3 μm). Energy-dispersive spectroscopy spectra were acquired on aggregates on each scaffold. Phosphorus (2.013 keV) and calcium (3.69 keV) peaks are indicated on each spectrum. Three different scaffolds were analyzed for each experimental condition;

FIG. 7 shows coating of biomaterial (disk shape) with alternate solution of calcium chloride and disodium phosphate. The number of the top left corner indicates the number of incubation cycles;

FIG. 8 shows cylinder-shaped biomaterial. Non-coated graft (A); Pre-coated graft after a 4-week subcutaneous implantation in rat (B) (N=3 implants in 1 rat); Ct scan of graft after a 4-week subcutaneous implantation in rat (C) (N=3 implants in 1 rat);

FIG. 9 shows histological staining of a disk-shaped, pre-coated biomaterial. Hematoxylin and Eosin (A-C), Masson Trichrome (D-F) and Von Kossa/Van Geisson (G-I);

FIG. 10 shows histological staining of a cylindrical-shaped, pre-coated biomaterial (transverse cut). Hematoxylin and Eosin (A-C), Masson Trichrome (D-F), and Von Kossa/Van Geisson (G-I);

FIG. 11 shows a hanging membrane (decellularized orange pith) glued and sandwiched between decellularized apple hypathium tissue;

FIG. 12 shows three-dimensional rendering of an implanted biomaterial (with perforations) in critical-size defects at 4 weeks (A) and 8 weeks (B);

FIG. 13 shows bone volume fraction over total volume inside the defect. The cylindrical region of interest were obtained by fitting a cylinder with approximatively the same dimensions as the defect, in CT scan slices. N=6 defects (3 animals) for the 4 week-time point and N=6 defects (3 animals) for the 8 week-time point;

FIG. 14 shows a dislocation experiment. Typical force vs distance and force-displacement curves obtained during push-out experiments are shown in (A). The dislocation is taken as the approximative maximum force in the force vs distance graph (red arrow). Push-out device with specimen is shown in (B) left and right, providing photographs of uniaxial compression device (Asterix indicates the load cell; Arrow indicates the sample);

FIG. 15 shows representative images of implanted scaffolds histological cross-sections at 8 weeks as described in Example 4. Sections were stained with either hematoxylin and eosin (H&E) or Goldner's Trichrome (GTC). Arrows indicates red blood cells. Presence of collagen is visible at 8 weeks (scale bar=1 mm and 200 μm for the insets);

FIG. 16 shows histological sections aftert 4 weeks after implantation (4WCH2). Hematoxylin and Eosin staining is shown in (A), Von Kossa/Van Gieson staining is shown in (B) and Masson Goldner Trichrome staining is shown in (C). Scale=2 mm for (A), (B) and (C);

FIG. 17 shows histological section at after 8 weeks after implantation (8WCH1). Hematoxylin and Eosin staining is shown in (A), Von Kossa/Van Gieson staining is shown in (B) and Masson Goldner Trichrome staining is shown in (C). Scale=2 mm for (A), (B) and (C);

FIG. 18 shows implantation in a rat critical size calvarial defect model. Perforated 5 mm diameter by 1 mm thickness biomaterial is shown in (A). Implantation of the biomaterial into bilateral defects is shown in (B). On the left, the biomaterial is implanted, empty defect on the right-hand side. Rat ID: 4WME. (A) shows scaffold implants and (B) shows exposed skull with bilateral defects (arrow indicates implanted site);

FIG. 19 shows tissue removal after 8-week implantation. A view prior to the complete resection of the calvaria is shown in (A); the top view of the resected calvaria is shown in (B); and the bottom view of the resected calvaria is shown in (C);

FIG. 20A-D shows interlocked composite of apples and carrots (SCC);

FIG. 21 shows Alizarin Red S staining for calcium deposition in MC3T3 E1 cell-laden composites as described in Example 5. Left to right: hyaluronic acid and decellularized apple (pre-differentiation), alginate and decellularized apple (pre-differentiation), hyaluronic acid and decellularized apple (post-differentiation), alginate and decellularized apple (post-differentiation).

FIG. 22 shows Alkaline phosphatase staining with BCIP NBT SigmaFast™ tablets in MC3T3 E1 cell-laden composites as described in Example 5. Left to right: hyaluronic acid and decellularized apple (pre-differentiation), alginate and decellularized apple (pre-differentiation), hyaluronic acid and decellularized apple (post-differentiation), alginate and decellularized apple (post-differentiation);

FIG. 23 shows (A) Cyclic hydrostatic pressure device schematics as described in Example 6. Hydrostatic pressure was applied by modulating the pressure in the gas phase above the culture wells in a custom-build pressure chamber. Air from incubator atmosphere was compressed using a compressor and injected in the pressure chamber using solenoid valves. (B) shows experimental conditions as described in Example 6. After 1 week of proliferation, cyclic hydrostatic pressure stimulation was applied during 1 hour per day, for up to 2 weeks at a frequency 1 Hz, oscillating between 0 and 280 kPa with respect to ambient pressure. The samples were removed from the pressure chamber after each cycle and kept at ambient pressure between the stimulation phases;

FIG. 24 shows cellular density after 1 week or 2 weeks of stimulation as described in Example 6. Statistical significance (* indicates p<0.05) was determined using a one-way ANOVA and Tukey post-hoc tests. Data are presented as means±S.E.M. of three replicate samples per condition, with three areas per sample. The results reveal that after 2 weeks in culture, there are significantly more cells present on scaffolds which experienced cyclic pressure loading compared to controls;

FIG. 25 shows alkaline phosphatase (ALP) activity after 1 week or 2 weeks of stimulation as described in Example 6. Statistical significance (* indicates p<0.05) was determined using a one-way ANOVA and Tukey post-hoc tests. Data are presented as means±S.E.M. of three replicate samples per condition. The results reveal that after 2 weeks in culture, there is significantly ALP activity (a marker of differentiation) in cells present on scaffolds which experienced cyclic pressure loading compared to controls;

FIG. 26 shows mineral deposit quantification with Alizarin Red S (ARS) staining after 1 week or 2 weeks of stimulation as described in Example 6. Statistical significance (* indicates p<0.05) was determined using a one-way ANOVA and Tukey post-hoc tests. Data are presented as means±S.E.M. of three replicate samples per condition. The results reveal that after 2 weeks in culture, there is significantly more mineralization of the scaffolds which experienced cyclic pressure loading compared to controls;

FIG. 27 shows Young's modulus of decellularized AA with hyaluronic acid (HA) or alginate hydrogels without cells (control) and with cells after differentiation (Diff) as described in Example 5;

FIG. 28 shows representative confocal laser scanning microscope image showing seeded cells scaffolds (scale bar=100 μm—applies to all). The scaffolds were stained for cellulose (red) and for cell nuclei (blue) as described in FIG. 24, and in Example 6; and

FIG. 29 shows Young's modulus of scaffolds after 1 week or 2 weeks of stimulation as described in Example 6. Statistical significance (* indicates p<0.05) was determined using a one-way ANOVA and Tukey post-hoc tests. Data are presented as means±S.E.M. of three replicate samples per condition.

### DETAILED DESCRIPTION

Described herein are scaffold biomaterials, methods for the preparation thereof, as well as methods and uses thereof in a variety of applications including, for example, bone tissue engineering (BTE). It will be appreciated that embodiments and examples are provided for illustrative purposes intended for those skilled in the art, and are not meant to be limiting in any way.

Provided herein are materials (biomaterials) that may be used in BTE applications, such as in the repair and/or regeneration of damaged, degraded, defective, and/or missing bone structures. The present inventors have now developed scaffold biomaterials comprising decellularized plant or fungal tissue, wherein the decellularized plant or fungal tissue may optionally be at least partially coated or mineralized (with, for example, apatite), wherein the scaffold biomaterial may optionally further include a protein-based hydrogel (such as, for example, a collagen hydrogel) and/or a polysaccharide-based hydrogel (such as, for example, an agarose or agarose-based gel/hydrogel, or an alginate or alginate-based gel/hydrogel, or a hyaluronic acid or hyaluronic acid-based gel/hydrogel), or both. Experimental studies described herein indicate that such scaffold biomaterials may be biocompatible, and may support growth of pre-osteoblasts, which may be differentiated in the scaffold biomaterials. Accordingly, scaffold biomaterials as described herein may be used for BTE, such as in the repair and/or regeneration of damaged, degraded, defective, and/or missing bone structures, for example. Results indicate that protein-based hydrogels, such as collagen hydrogels, may be used in such scaffold biomaterials, and that pre-mineralization of scaffold biomaterials with, for example, hydroxyapatite may be used.

Scaffold Biomaterials

In an embodiment, there is provided herein a scaffold biomaterial comprising:
a decellularized plant or fungal tissue from which cellular materials and nucleic acids of the tissue are removed, the decellularized plant or fungal tissue comprising a 3-dimensional porous structure; and
a protein-based hydrogel, a polysaccharide-based hydrogel, or both.

In certain embodiments, the protein-based hydrogel may comprise any suitable hydrogel comprising one or more proteins or derivatives thereof. In certain embodiments, the protein-based hydrogel may comprise collagen, osteonectin, osteopontin, bone sialoprotein, osteocalcin, fibronectin, laminin, a proteoglycan, bone morphogenetic protein, other matrix protein(s), or any combinations thereof. In certain embodiments, the protein-based hydrogel may comprise a collagen hydrogel. In certain embodiments, the protein-based hydrogel may comprise collagen I.

In certain embodiments, the polysaccharide-based hydrogel may comprise any suitable hydrogel comprising one or more carbohydrates or polysaccharides or derivatives thereof. In certain embodiments, the hydrogel may comprise an agarose-based gel/hydrogel, or another carbohydrate-based hydrogel.

In certain embodiments, the decellularized plant or fungal tissue and/or the protein-based hydrogel and/or the polysaccharide-based hydrogel may comprise one or more markers of osteogenic differentiation, such as osteonectin, osteopontin, bone sialoprotein, osteocalcin, fibronectin, laminin, a proteoglycan, or any combinations thereof. In certain embodiments, the decellularized plant or fungal tissue and/or the protein-based hydrogel and/or the polysaccharide-based hydrogel may comprise one or more proteins found in normal bone matrix.

In another embodiment, there is provided herein a scaffold biomaterial comprising:
a decellularized plant or fungal tissue from which cellular materials and nucleic acids of the tissue are removed, the decellularized plant or fungal tissue comprising a 3-dimensional porous structure;
the decellularized plant or fungal tissue being at least partially coated or mineralized.

In certain embodiments, the decellularized plant or fungal tissue may be at least partially coated or mineralized with one or more phosphate minerals. In certain embodiments, the decellularized plant or fungal tissue may be at least partially coated or mineralized with apatite, osteocalcium phosphate, a biocompatible ceramic, a biocompatible glass, a biocompatible metal nanoparticle, nanocrystalline cellulose, or any combinations thereof. In certain embodiments, the decellularized plant or fungal tissue may be at least partially coated or mineralized with apatite. In certain embodiments, the apatite may comprise hydroxyapatite. In certain embodiments, the decellularized plant or fungal tissue may be at least partially coated or mineralized with nanocrystalline cellulose to increase stiffness of the decellularized plant or fungal tissue.

In still another embodiment, there is provided herein a scaffold biomaterial comprising:
a decellularized plant or fungal tissue from which cellular materials and nucleic acids of the tissue are removed, the decellularized plant or fungal tissue comprising a 3-dimensional porous structure, and the decellularized plant or fungal tissue being at least partially coated or mineralized; and
a protein-based hydrogel, a polysaccharide-based hydrogel, or both.

In certain embodiments, the decellularized plant or fungal tissue may be at least partially coated or mineralized with one or more phosphate minerals. In certain embodiments, the decellularized plant or fungal tissue may be at least partially coated or mineralized with apatite, osteocalcium phosphate, a biocompatible ceramic, a biocompatible glass, a biocompatible metal nanoparticle, nanocrystalline cellulose, or any combinations thereof. In certain embodiments, the decellularized plant or fungal tissue may be at least partially coated or mineralized with apatite. In certain embodiments, the apatite may comprise hydroxyapatite. In certain embodiments, the decellularized plant or fungal tissue may be at least partially coated or mineralized with nanocrystalline cellulose to increase stiffness of the decellularized plant or fungal tissue.

In certain embodiments, the protein-based hydrogel may comprise any suitable hydrogel comprising one or more proteins or derivatives thereof. In certain embodiments, the protein-based hydrogel may comprise collagen, osteonectin, osteopontin, bone sialoprotein, osteocalcin, fibronectin, laminin, a proteoglycan, bone morphogenetic protein, other matrix protein(s), or any combinations thereof. In certain embodiments, the protein-based hydrogel may comprise a collagen hydrogel. In certain embodiments, the protein-based hydrogel may comprise collagen I.

In certain embodiments, the polysaccharide-based hydrogel may comprise any suitable hydrogel comprising one or more carbohydrates or polysaccharides or derivatives thereof. In certain embodiments, the hydrogel may comprise an agarose-based hydrogel, or another carbohydrate-based hydrogel.

In certain embodiments, the decellularized plant or fungal tissue and/or the protein-based hydrogel and/or the polysaccharide-based hydrogel may comprise one or more markers of osteogenic differentiation, such as osteonectin, osteopontin, bone sialoprotein, osteocalcin, fibronectin, laminin, a proteoglycan, or any combinations thereof. In certain embodiments, the decellularized plant or fungal tissue and/or the protein-based hydrogel and/or the polysaccharide-based hydrogel may comprise one or more proteins found in normal bone matrix.

In certain embodiments, the biomaterials described herein may be derived from cell wall architectures and/or vascular structures found in the plant and fungus kingdoms to create 3D scaffolds which may promote cell infiltration, cell growth, bone tissue repair, and/or bone reconstruction, etc. As will be understood, biomaterials as described herein may be produced from any suitable part of plant or fungal organisms. Biomaterials may comprise, for example, substances such as cellulose, chitin, lignin, hemicellulose, pectin, and/or any other suitable biochemicals/biopolymers which are naturally found in these organisms.

As will be understood, unless otherwise indicated, the meaning/definition of plant and fungi kingdoms used herein is based on the Cavalier-Smith classification (1998).

In certain embodiments, the plant or fungal tissue may comprise generally any suitable plant or fungal tissue or part containing a suitable scaffold structure appropriate for the particular application.

In certain embodiments of the scaffold material or materials above, the plant or fungal tissue may comprise an apple hypanthium (Malus pumila) tissue, a fern (Monilophytes) tissue, a turnip (Brassica rapa) root tissue, a gingko branch tissue, a horsetail (equisetum) tissue, a hermocallis hybrid leaf tissue, a kale (Brassica oleracea) stem tissue, a conifers Douglas Fir (Pseudotsuga menziesii) tissue, a cactus fruit (pitaya) flesh tissue, a Maculata Vinca tissue, an Aquatic Lotus (Nelumbo nucifera) tissue, a Tulip (Tulipa gesneriana) petal tissue, a Plantain (Musa paradisiaca) tissue, a broccoli (Brassica oleracea) stem tissue, a maple leaf (Acer psuedoplatanus) stem tissue, a beet (Beta vulgaris) primary root tissue, a green onion (Allium cepa) tissue, a orchid (Orchidaceae) tissue, turnip (Brassica rapa) stem tissue, a leek (Allium ampeloprasum) tissue, a maple (Acer) tree branch tissue, a celery (Apium graveolens) tissue, a green onion (Allium cepa) stem tissue, a pine tissue, an aloe vera tissue, a watermelon (Citrullus lanatus var. lanatus) tissue, a Creeping Jenny (Lysimachia nummularia) tissue, a cactae tissue, a Lychnis Alpina tissue, rhubarb (Rheum rhabarbarum) tissue, a pumpkin flesh (Cucurbita pepo) tissue, a Dracena (Asparagaceae) stem tissue, a Spiderwort (Tradescantia virginiana) stem tissue, an Asparagus (Asparagus officinalis) stem tissue, a mushroom (Fungi) tissue, a fennel (Foeniculum vulgare) tissue, a rose (Rosa) tissue, a carrot (Daucus carota) tissue, or a pear (Pomaceous) tissue. Additional examples of plant and fungal tissues are described in Example 18 of WO2017/136950, entitled “Decellularised Cell Wall Structures from Plants and Fungus and Use Thereof as Scaffold Materials”, herein incorporated by reference in its entirety.

As will also be understood, cellular materials and nucleic acids of the plant or fungal tissue may include intracellular contents such as cellular organelles (e.g. chloroplasts, mitochondria), cellular nuclei, cellular nucleic acids, and/or cellular proteins. These may be substantially removed, partially removed, or fully removed from the plant or fungal tissue, and/or from the scaffold biomaterial. It will recognized that trace amounts of such components may still be present in the decellularised plant or fungal tissues described herein. As will also be understood, references to decellularized plant or fungal tissue herein are intended to reflect that such cellular materials found in the plant or fungal source of the tissues have been substantially removed—this does not preclude the possibility that the decellularized plant or fungal tissue may in certain embodiments contain or comprise subsequently introduced, or reintroduced, cells, cellular materials, and/or nucleic acids of generally any kind, such as animal or human cells, such as bone or bone progenitor cells/tissues.

Various methods may be used for producing scaffold biomaterials as described herein. By way of example, in certain embodiments of the scaffold biomaterials above, the decellularised plant or fungal tissue may comprise plant or fungal tissue(s) which have been decellularised by thermal shock, treatment with detergent (e.g. SDS, Triton X, EDA, alkyline treatment, acid, ionic detergent, non-ionic detergents, and zwitterionic detergents), osmotic shock, lyophilisation, physical lysing (e.g. hydrostatic pressure), electrical disruption (e.g. non thermal irreversible electroporation), or enzymatic digestion, or any combination thereof. In certain embodiments, biomaterials as described herein may be obtained from plants and/or fungi by employing decellularization processes which may comprise any of several approaches (either individually or in combination) including, but not limited to, thermal shock (for example, rapid freeze thaw), chemical treatment (for example, detergents), osmotic shock (for example, distilled water), lyophilisation, physical lysing (for example, pressure treatment), electrical disruption and/or enzymatic digestion.

In certain embodiments, the decellularised plant or fungal tissue may comprise plant or fungal tissue which has been decellularised by treatment with a detergent or surfactant. Examples of detergents may include, but are not limited to sodium dodecyl sulphate (SDS), Triton X, EDA, alkyline treatment, acid, ionic detergent, non-ionic detergents, and zwitterionic detergents.

In still further embodiments, the decellularised plant or fungal tissue may comprise plant or fungal tissue which has been decellularised by treatment with SDS. In still another embodiment, residual SDS may be removed from the plant or fungal tissue by washing with an aqueous divalent salt solution. The aqueous divalent salt solution may be used to precipitate/crash a salt residue containing SDS micelles out of the solution/scaffold, and a dH₂O, acetic acid or dimethylsulfoxide (DMSO) treatment, or sonication, may have been used to remove the salt residue or SDS micelles. In certain embodiments, the divalent salt of the aqueous divalent salt solution may comprise, for example, MgCl₂ or CaCl₂.

In another embodiment, the plant or fungal tissue may be decellularised by treatment with an SDS solution of between 0.01 to 10%, for example about 0.1% to about 1%, or, for example, about 0.1% SDS or about 1% SDS, in a solvent such as water, ethanol, or another suitable organic solvent, and the residual SDS may have been removed using an aqueous CaCl₂ solution at a concentration of about 100 mM followed by incubation in dH₂O. In certain embodiments, the SDS solution may be at a higher concentration than 0.1%, which may facilitate decellularisation, and may be accompanied by increased washing to remove residual SDS. In particular embodiments, the plant or fungal tissue may be decellularised by treatment with an SDS solution of about 0.1% SDS in water, and the residual SDS may have been removed using an aqueous CaCl₂ solution at a concentration of about 100 mM followed by incubation in dH2O.

Further examples of decellularization protocols which may be adapted for producing decellularized plant or fungal tissue for scaffold biomaterials as described herein may be found in WO2017/136950, entitled “Decellularised Cell Wall Structures from Plants and Fungus and Use Thereof as Scaffold Materials”, herein incorporated by reference in its entirety.

In certain embodiments, the scaffold biomaterials as described herein may comprise decellularized plant or fungal tissue comprising a pore size of about 100 to about 200 μm, or of about 150 to about 200 μm. In certain embodiments, the scaffold biomaterial may comprise a Young's moduli between about 20 kPa to about 1 MPa. In certain embodiments, the decellularized plant or fungal tissue may comprise decellularized apple, such as decellularized apple hypanthium tissue.

In certain embodiments, the scaffold biomaterials as described herein may comprise a polysaccharide-based hydrogel and/or a protein-based hydrogel, such as a collagen hydrogel, which may be soaked into and/or permeate through the 3D porous structure of the decellularized plant or fungal tissue, may be coated on or surrounding the decellularized plant or fungal tissue, or a combination thereof.

As will be understood, in certain embodiments, a hydrogel as described herein may include any suitable dilute 3D cross-linked system comprising water as a primary component, which may be substantially non-flowable. In certain embodiments, cross-linking may provide shape/mechanical stability to the hydrogel. In certain embodiments, the hydrogel may be reinforced by creating it around scaffold biomaterials and/or decellularized plant or fungal tissue. In certain embodiments, hydrogels as described herein may comprise one or more ECM proteins, hyaluronic acid, or both, for example. Various hydrogels will be known to the person of skill in the art having regard to the teachings herein. In certain embodiments, hydrogel viscoelastic properties may be tuned to create non-newtonian hydrogels which may stiffen under mechanical strain at low frequencies (i.e. strain harden during walking, to mechanically stimulate cells and provide structure for growing bone, for example). In certain embodiments, it is contemplated that hydrogels may be non-cross-linked, and may instead comprise entangled polymers, for example.

In certain embodiments, the collagen hydrogel may comprise collagen I.

In certain embodiments, the scaffold biomaterial may comprise one or more bone-relevant cell types such as preosteoblasts, osteoblasts, osteoclasts, and/or mesenchymal stem cells, or any combinations thereof. In another embodiment, the scaffold biomaterial may be pre-seeded with one or more bone-relevant cell types such as preosteoblasts, osteoblasts, osteoclasts, and/or mesenchymal stem cells, or any combinations thereof. In certain embodiments of the scaffold biomaterials as described herein, pore walls of the decellularized plant or fungal tissue may be mineralized by the osteoblasts.

In certain embodiments, the hydrogel may comprise bone progenitor cells, or bone or bone tissue cells, such as but not limited to pre-osteoblasts and/or osteoblasts, for example. In certain embodiments, stem cells (such as mesenchymal, skeletal, or other stem cells) may be added to the hydrogel and/or otherwise added to the scaffold biomaterials. In certain embodiments, the hydrogel may comprise osteocalcium phosphate, a biocompatible ceramic, a biocompatible glass, a biocompatible metal nanoparticle, nanocrystalline cellulose, or any combinations thereof. In certain embodiments, the hydrogel may comprise apatite, such as hydroxyapatite.

In certain embodiments, the decellularized plant or fungal tissue of the scaffold biomaterials as described herein may be at least partially coated or mineralized. In certain embodiments, the decellularized plant or fungal tissue may be at least partially coated or mineralized with one or more phosphate minerals. In certain embodiments, the decellularized plant or fungal tissue may be at least partially coated or mineralized with apatite, osteocalcium phosphate, a biocompatible ceramic, a biocompatible glass, a biocompatible metal nanoparticle, nanocrystalline cellulose, or any combinations thereof. In certain embodiments, the decellularized plant or fungal tissue may be at least partially coated or mineralized with apatite. In certain embodiments, the apatite may comprise hydroxyapatite. In certain embodiments, the decellularized plant or fungal tissue may be at least partially coated or mineralized with nanocrystalline cellulose to increase stiffness of the decellularized plant or fungal tissue. In certain embodiments, the decellularized plant or fungal tissue may be at least partially coated or mineralized with apatite, such as hydroxyapatite.

In certain embodiments, it is contemplated that the decellularized plant or fungal tissue may be at least partially coated or mineralized via any of a variety of suitable techniques. By way of example, in certain embodiments, the decellularized plant or fungal tissue may be at least partially coated or mineralized with apatite, for example, by alternating exposure to solutions of calcium chloride and disodium phosphate. In certain embodiments, it is contemplated that the decellularized plant or fungal tissue may be at least partially coated or mineralized via immersion in simulated body fluid; thermal spraying; sputter coating; sol-gel deposition; hot isostatic pressing; dip coating; electrospinning; or any combinations thereof. Examples of coating or mineralizing techniques are described in Shin et al., 2017, Biomimetic Mineralization of Biomaterials Using Simulated Body Fluids for Bone Tissue Engineering and Regenerative Medicine, Tissue Engineering Part A, 23:19-20, https://dx.doi.org/10.1089%2Ften.tea.2016.0556, which is herein incorporated by reference in its entirety.

In certain embodiments, the decellularized plant or fungal tissue is cellulose-based, chitin-based, chitosan-based, lignin-based, hemicellulose-based, or pectin-based, or any combination thereof. In certain embodiments, the plant or fungal tissue may comprise a tissue from apple hypanthium (Malus pumila) tissue, a fern (Monilophytes) tissue, a turnip (Brassica rapa) root tissue, a gingko branch tissue, a horsetail (equisetum) tissue, a hermocallis hybrid leaf tissue, a kale (Brassica oleracea) stem tissue, a conifers Douglas Fir (Pseudotsuga menziesii) tissue, a cactus fruit (pitaya) flesh tissue, a Maculata Vinca tissue, an Aquatic Lotus (Nelumbo nucifera) tissue, a Tulip (Tulipa gesneriana) petal tissue, a Plantain (Musa paradisiaca) tissue, a broccoli (Brassica oleracea) stem tissue, a maple leaf (Acer psuedoplatanus) stem tissue, a beet (Beta vulgaris) primary root tissue, a green onion (Allium cepa) tissue, a orchid (Orchidaceae) tissue, turnip (Brassica rapa) stem tissue, a leek (Allium ampeloprasum) tissue, a maple (Acer) tree branch tissue, a celery (Apium graveolens) tissue, a green onion (Allium cepa) stem tissue, a pine tissue, an aloe vera tissue, a watermelon (Citrullus lanatus var. lanatus) tissue, a Creeping Jenny (Lysimachia nummularia) tissue, a cactae tissue, a Lychnis Alpina tissue, a rhubarb (Rheum rhabarbarum) tissue, a pumpkin flesh (Cucurbita pepo) tissue, a Dracena (Asparagaceae) stem tissue, a Spiderwort (Tradescantia virginiana) stem tissue, an Asparagus (Asparagus officinalis) stem tissue, a mushroom (Fungi) tissue, a fennel (Foeniculum vulgare) tissue, a rose (Rosa) tissue, a carrot (Daucus carota) tissue, or a pear (Pomaceous) tissue, or a genetically altered tissue produced via direct genome modification or through selective breeding, or any combinations thereof.

In certain embodiments of the scaffold biomaterials as described herein, the scaffold biomaterials may further comprise living cells, in particular non-native cells, on and/or within the decellularized plant or fungal tissue. In certain embodiments, the living cells may be animal cells. In certain embodiments, the living cells may be mammalian cells. In certain embodiments, the living cells may be human cells.

In certain embodiments, the scaffold biomaterials as described herein may comprise two or more scaffold subunits which are glued, cross-linked, or interlocked together. In certain embodiments of the scaffold biomaterials as described herein, the decellularized plant or fungal tissue may comprise two or more different decellularized plant or fungal tissues derived from different tissues or different sources. In certain embodiments, the two or more different decellularized plant or fungal tissues may be glued, cross-linked, or interlocked together.

In another embodiment, there is provided herein a scaffold biomaterial as described herein for use in bone tissue engineering. In still another embodiment, there is provided herein a bone graft comprising a scaffold biomaterial as described herein. In another embodiment, there is provided herein a BTE implant comprising a scaffold biomaterial as described herein.

In certain embodiments, unlike many commercial biomaterials, plant/fungus derived biomaterials as described herein may be substantially non-resorbable or poorly resorbable (i.e. they will not substantially breakdown and be absorbed by the body). The non-resorbable characteristic of these scaffolds may offer certain benefits. For example, in certain embodiments, biomaterials described herein may be resistant to shape change, and/or may hold their intended geometry over long periods of time. In certain embodiments, since they may have a minimal footprint compared to certain other products, they may be considered effectively invisible to the body, eliciting almost no immune response. In some cases, when some resorbable biomaterials break down, their by-products may illicit an adverse immune response, as well as induce oxidative stress and result in an increase of pH in the recovering tissue, which may be avoided by using a non-resorbable biomaterial.

Indeed, in certain embodiments, the decellularized plant or fungal tissues and/or scaffold biomaterials as described herein may further comprise living cells on and/or within the scaffold biomaterials. In certain embodiments, the living cells may be animal cells, mammalian cells, or human cells. In certain embodiments, the living cells may comprise pre-osteoblasts, osteoblasts, and/or other bone or bone tissue-related cells.

In certain embodiments, the plant or fungal tissue may be genetically altered via direct genome modification or through selective breeding, to create an additional plant or fungal architecture which may be configured to physically mimic a tissue and/or to functionally promote a target tissue effect, particularly bone tissues and bone engineering effects. The skilled person having regard to the teachings herein will be able to select a suitable scaffold biomaterial to suit a particular application. In certain embodiments, a suitable tissue may be selected for a particular application based on, for example, physical characteristics such as size, structure (porous/tubular), stiffness, strength, hardness and/or ductility, which may be measured and matched to a particular application.

Moreover, chemical properties such as reactivity, coordination number, enthalpy of formation, stability, toxicity, and/or types of bonds may also be considered for selection to suit a particular application. Such characteristics (physical and chemical) may also be directly modified before or after decellularization and/or functionalization to respond to the specific application.

In certain embodiments, scaffold biomaterials may be sourced from the same tissue or part of the plant or fungus, or from different parts or tissues of the plant or fungus. In certain embodiments, scaffold biomaterials may be sourced from the same individual plant or fungus, or from multiple plants or fungi of the same species. In certain embodiments, the scaffold biomaterials may be sourced from plants or fungi of different species, such that the scaffold comprises structures from more than one species. In certain embodiments, the scaffold biomaterials may be selected so as to provide particular features. For example, in certain embodiments, scaffold biomaterials having porosity and/or rigidity falling within a certain range may be selected, so as to mimic natural tissues and/or structures involved in bone tissue regeneration, repair, and/or engineering. In certain embodiments, the plant or fungal tissue may comprise apple, or apple hypanthium, tissue, or another plant or fungal tissue having similar porosity and/or rigidity characteristic(s).

In certain embodiments, the scaffold biomaterial may be a scaffold biomaterial configured to physically mimic a tissue of the subject and/or to functionally promote a target tissue effect in the subject. Methods of using such scaffold biomaterials as are described herein may, in certain embodiments, include a step of selecting a scaffold biomaterial as described herein for which the decellularised plant or fungal tissue is configured to physically mimic a tissue of the subject and/or to functionally promote a target tissue effect in the subject. As will be understood, the tissue will typically be a bone-related tissue, and the target tissue effect will typically be a bone regeneration, repair, growth, and/or bone engineering effect. The skilled person having regard to the teachings herein will be able to select a suitable scaffold biomaterial to suit a particular application.

In certain embodiments, the decellularized plant or fungal tissue and/or scaffold biomaterials as described herein may further comprise living cells on and/or within the plant or fungal tissue. In certain embodiments, the living cells may be animal cells, mammalian cells, or human cells. In certain embodiments, the cells may be cells introduced or seeded into and/or onto the scaffold biomaterials and/or decellularized plant or fungal tissue, or may be cells infiltrating into or onto the scaffold biomaterials and/or decellularized plant or fungal tissue following implantation of the scaffold biomaterials and/or decellularized plant or fungal tissue into a living animal or plant subject, for example. In certain embodiments, the living cells may comprise bone tissue cells, or bone progenitor cells. In certain embodiments, the living cells may comprise pre-osteoblasts, or osteoblasts.

In another embodiment, there is provided herein a kit comprising any one or more of:
a decellularized plant or fungal tissue from which cellular materials and nucleic acids of the tissue are removed, the decellularized plant or fungal tissue comprising a 3-dimensional porous structure;
a protein-based hydrogel;
a polysaccharide-based hydrogel;
apatite;
calcium chloride;
disodium phosphate;
osteocalcium phosphate;
a biocompatible ceramic;
a biocompatible glass;
a biocompatible metal nanoparticle;
nanocrystalline cellulose;
mammalian cells, such as preosteoblasts, osteoblasts, differentiated bone and/or calvaria tissue cells, or any combination thereof;
plant or fungal tissue, decellularization reagents, or both;
a buffer; and/or
instructions for performing any of the method or methods as described herein.

In certain embodiments, the protein-based hydrogel may comprise collagen, osteonectin, osteopontin, bone sialoprotein, osteocalcin, fibronectin, laminin, a proteoglycan, bone morphogenetic protein, other matrix protein(s), or any combinations thereof. In certain embodiments, the protein-based hydrogel may comprise a collagen hydrogel. In certain embodiments, the protein-based hydrogel may comprise collagen I. In certain embodiments, the polysaccharide-based hydrogel may comprise an agarose-based gel/hydrogel, alginate-based gel/hydrogel, a hyaluronic acid-based gel/hydrogel, or another carbohydrate-based hydrogel. In certain embodiments, the apatite may comprise hydroxyapatite. In certain embodiments, the decellularized plant or fungal tissue and/or the protein-based hydrogel and/or the polysaccharide-based hydrogel may comprise one or more markers of osteogenic differentiation, such as osteonectin, osteopontin, bone sialoprotein, osteocalcin, fibronectin, laminin, a proteoglycan, or any combinations thereof. In certain embodiments, the decellularized plant or fungal tissue and/or the protein-based hydrogel and/or the polysaccharide-based hydrogel may comprise one or more proteins found in normal bone matrix.

Methods of Production, and Methods and Uses of Scaffold Biomaterials

In another embodiment, there is provided herein a method for producing a scaffold biomaterial, said method comprising:
providing a decellularized plant or fungal tissue from which cellular materials and nucleic acids of the tissue are removed, the decellularized plant or fungal tissue comprising a 3-dimensional porous structure; and
introducing a protein-based hydrogel, a polysaccharide-based hydrogel, or both, into the decellularized plant or fungal tissue.

In certain embodiments, the protein-based hydrogel and/or the polysaccharide-based hydrogel may be introduced into the decellularized plant or fungal tissue by any suitable technique known to the person of skill in the art having regard to the teachings herein. In certain embodiments, the protein-based hydrogel and/or the polysaccharide-based hydrogel may be introduced into the decellularized plant or fungal tissue by immersion, pouring, molding, under an electric field, guided lithography, or electrospinning, for example.

In certain embodiments, the protein-based hydrogel may comprise any suitable hydrogel comprising one or more proteins or derivatives thereof. In certain embodiments, the protein-based hydrogel may comprise collagen, osteonectin, osteopontin, bone sialoprotein, osteocalcin, fibronectin, laminin, a proteoglycan, bone morphogenetic protein, other matrix protein(s), or any combinations thereof. In certain embodiments, the protein-based hydrogel may comprise a collagen hydrogel. In certain embodiments, the protein-based hydrogel may comprise collagen I.

In certain embodiments, the polysaccharide-based hydrogel may comprise any suitable hydrogel comprising one or more carbohydrates or polysaccharides or derivatives thereof. In certain embodiments, the hydrogel may comprise an agarose-based hydrogel, alginate-based hydrogel, hyaluronic acid-based hydrogel, or another carbohydrate-based hydrogel.

In certain embodiments, the decellularized plant or fungal tissue and/or the protein-based hydrogel and/or the polysaccharide-based hydrogel may comprise one or more markers of osteogenic differentiation, such as osteonectin, osteopontin, bone sialoprotein, osteocalcin, fibronectin, laminin, a proteoglycan, or any combinations thereof. In certain embodiments, the decellularized plant or fungal tissue and/or the protein-based hydrogel and/or the polysaccharide-based hydrogel may comprise one or more proteins found in normal bone matrix.

In yet another embodiment, there is provided herein a method for producing a scaffold biomaterial, said method comprising:
providing a decellularized plant or fungal tissue from which cellular materials and nucleic acids of the tissue are removed, the decellularized plant or fungal tissue comprising a 3-dimensional porous structure; and
at least partially coating or mineralizing the decellularized plant or fungal tissue.

In certain embodiments, the decellularized plant or fungal tissue may be at least partially coated or mineralized with one or more phosphate minerals. In certain embodiments, the decellularized plant or fungal tissue may be at least partially coated or mineralized with apatite, osteocalcium phosphate, a biocompatible ceramic, a biocompatible glass, a biocompatible metal nanoparticle, nanocrystalline cellulose, or any combinations thereof. In certain embodiments, the decellularized plant or fungal tissue may be at least partially coated or mineralized with apatite. In certain embodiments, the apatite may comprise hydroxyapatite. In certain embodiments, the decellularized plant or fungal tissue may be at least partially coated or mineralized with nanocrystalline cellulose to increase stiffness of the decellularized plant or fungal tissue. In certain embodiments, the apatite may comprise hydroxyapatite.

In certain embodiments, the step of coating or mineralizing the decellularized plant or fungal tissue comprises subjecting the decellularized plant or fungal tissue to alternating exposures to solutions of calcium chloride and disodium phosphate.

In certain embodiments, it is contemplated that the decellularized plant or fungal tissue may be at least partially coated or mineralized via any of a variety of suitable techniques. By way of example, in certain embodiments, the decellularized plant or fungal tissue may be at least partially coated or mineralized with apatite, for example, by alternating exposure to solutions of calcium chloride and disodium phosphate. In certain embodiments, it is contemplated that the decellularized plant or fungal tissue may be at least partially coated or mineralized via immersion in simulated body fluid; thermal spraying; sputter coating; sol-gel deposition; hot isostatic pressing; dip coating; electrospinning; or any combinations thereof. Examples of coating or mineralizing techniques are described in Shin et al., 2017, Biomimetic Mineralization of Biomaterials Using Simulated Body Fluids for Bone Tissue Engineering and Regenerative Medicine, Tissue Engineering Part A, 23:19-20, https://dx.doi.org/10.1089%2Ften.tea.2016.0556, which is herein incorporated by reference in its entirety.

In certain embodiments, the methods described herein may comprise both introducing a protein-based hydrogel and/or a polysaccharide-based hydrogel to the scaffold biomaterial, and mineralizing the decellularized plant or fungal tissue, providing a pre-mineralized scaffold biomaterial including a hydrogel coated and/or loaded therein.

In still another embodiment, the methods as described herein may further comprise a step of introducing living cells, in particular non-native cells, on and/or within the decellularized plant or fungal tissue. In certain embodiments, the living cells may comprise animal cells. In certain embodiments, the living cells may comprise mammalian cells. In certain embodiments, the living cells may comprise human cells. In certain embodiments, the living cells may comprise preosteoblasts, osteoblasts, differentiated bone and/or calvaria tissue cells, or any combination thereof.

Methods for the isolation and decellularization of plant or fungal tissue, and methods for preparing scaffold biomaterials are described in detail herein. As well, experimental examples of such methods are described in detail in the Examples section below.

Further examples of decellularization protocols which may be adapted for producing decellularized plant or fungal tissues for scaffold biomaterials as described herein may be found in WO2017/136950, entitled “Decellularised Cell Wall Structures from Plants and Fungus and Use Thereof as Scaffold Materials”, herein incorporated by reference in its entirety.

In still another embodiment of any of the above method or methods, the plant or fungal tissue may comprise a tissue from apple hypanthium (Malus pumila) tissue, a fern (Monilophytes) tissue, a turnip (Brassica rapa) root tissue, a gingko branch tissue, a horsetail (equisetum) tissue, a hermocallis hybrid leaf tissue, a kale (Brassica oleracea) stem tissue, a conifers Douglas Fir (Pseudotsuga menziesii) tissue, a cactus fruit (pitaya) flesh tissue, a Maculata Vinca tissue, an Aquatic Lotus (Nelumbo nucifera) tissue, a Tulip (Tulipa gesneriana) petal tissue, a Plantain (Musa paradisiaca) tissue, a broccoli (Brassica oleracea) stem tissue, a maple leaf (Acer psuedoplatanus) stem tissue, a beet (Beta vulgaris) primary root tissue, a green onion (Allium cepa) tissue, a orchid (Orchidaceae) tissue, turnip (Brassica rapa) stem tissue, a leek (Allium ampeloprasum) tissue, a maple (Acer) tree branch tissue, a celery (Apium graveolens) tissue, a green onion (Allium cepa) stem tissue, a pine tissue, an aloe vera tissue, a watermelon (Citrullus lanatus var. lanatus) tissue, a Creeping Jenny (Lysimachia nummularia) tissue, a cactae tissue, a Lychnis Alpina tissue, a rhubarb (Rheum rhabarbarum) tissue, a pumpkin flesh (Cucurbita pepo) tissue, a Dracena (Asparagaceae) stem tissue, a Spiderwort (Tradescantia virginiana) stem tissue, an Asparagus (Asparagus officinalis) stem tissue, a mushroom (Fungi) tissue, a fennel (Foeniculum vulgare) tissue, a rose (Rosa) tissue, a carrot (Daucus carota) tissue, or a pear (Pomaceous) tissue, or a genetically altered tissue produced via direct genome modification or through selective breeding, or any combinations thereof. In another embodiment, the plant or fungal tissue may comprise apple hypanthium. Additional examples of plant and fungal tissues are described in Example 18 of WO2017/136950, entitled “Decellularised Cell Wall Structures from Plants and Fungus and Use Thereof as Scaffold Materials”, herein incorporated by reference in its entirety.

Examples of decellularization protocols which may be adapted for producing decellularized plant or fungal tissues for scaffold biomaterials as described herein may be found in WO2017/136950, entitled “Decellularised Cell Wall Structures from Plants and Fungus and Use Thereof as Scaffold Materials”, herein incorporated by reference in its entirety.

Various methods may be used for decellularization. By way of example, in certain embodiments, decellularization may include decellularization by thermal shock, treatment with detergent (e.g. SDS, Triton X, EDA, alkyline treatment, acid, ionic detergent, non-ionic detergents, and zwitterionic detergents), osmotic shock, lyophilisation, physical lysing (e.g. hydrostatic pressure), electrical disruption (e.g. non thermal irreversible electroporation), or enzymatic digestion, or any combination thereof. In certain embodiments, decellularization processes may comprise any of several approaches (either individually or in combination) including, but not limited to, thermal shock (for example, rapid freeze thaw), chemical treatment (for example, detergents), osmotic shock (for example, distilled water), lyophilisation, physical lysing (for example, pressure treatment), electrical disruption and/or enzymatic digestion.

In certain embodiments, decellularization may comprise treatment with a detergent or surfactant. Examples of detergents may include, but are not limited to sodium dodecyl sulphate (SDS), Triton X, EDA, alkyline treatment, acid, ionic detergent, non-ionic detergents, and zwitterionic detergents.

In still further embodiments, the decellularised plant or fungal tissue may comprise plant or fungal tissue which has been decellularised by treatment with SDS. In still another embodiment, residual SDS may be removed from the plant or fungal tissue by washing with an aqueous divalent salt solution. The aqueous divalent salt solution may be used to precipitate/crash a salt residue containing SDS micelles out of the solution/scaffold, and a dH²O, acetic acid or dimethylsulfoxide (DMSO) treatment, or sonication, may have been used to remove the salt residue or SDS micelles. In certain embodiments, the divalent salt of the aqueous divalent salt solution may comprise, for example, MgCl₂ or CaCl₂.

In another embodiment, the plant or fungal tissue may be decellularised by treatment with an SDS solution of between 0.01 to 10%, for example about 0.1% to about 1%, or, for example, about 0.1% SDS or about 1% SDS, in a solvent such as water, ethanol, or another suitable organic solvent, and the residual SDS may have been removed using an aqueous CaCl₂ solution at a concentration of about 100 mM followed by incubation in dH₂O. In certain embodiments, the SDS solution may be at a higher concentration than 0.1%, which may facilitate decellularisation, and may be accompanied by increased washing to remove residual SDS. In particular embodiments, the plant or fungal tissue may be decellularised by treatment with an SDS solution of about 0.1% SDS in water, and the residual SDS may have been removed using an aqueous CaCl₂ solution at a concentration of about 100 mM followed by incubation in dH₂O.

While certain of the design considerations of the presently described scaffold biomaterials may be related to certain of those described for the scaffold biomaterials of WO2017/136950, entitled “Decellularised Cell Wall Structures from Plants and Fungus and Use Thereof as Scaffold Materials” (herein incorporated by reference in its entirety), the presently described biomaterials and may provide benefit arising from inclusion of one or more hydrogels, and/or inclusion of pre-mineralization, for example. Thus, the presently described biomaterials may be particularly advantageous for applications where bone tissue engineering, repair, regeneration, growth, and/or replacement is desired, for example.

In certain embodiments, biomaterials as described herein may have applications in biomedical laboratory research and/or clinical regenerative medicine in human and/or veterinary applications, for example. Such biomaterials may be effective as scaffolds which may be used as investigative tools for industrial/academic biomedical researchers, for biomedical implants and/or bone grafts, and/or in other suitable applications in which scaffolds may be used. In certain embodiments, scaffold biomaterials as described herein may be used for regeneration of bone. In certain embodiments, scaffold biomaterials as described herein may be used as simple or complex tissues. By way of example, scaffolds may be used to replace/regenerate bone tissues following accident, malformation, injury, or other damage to the bone.

In another embodiment, any of the above method or methods may further comprise a step of introducing living plant or animal cells to the plant or fungal tissue. In another embodiment, any of the above method or methods may further comprise a step of culturing the living plant or animal cells on and/or in the scaffold biomaterial. In an embodiment, the living cells may comprise mammalian cells, such as human cells. In certain embodiments, the cells may comprise one or more bone tissue cells such as, for example, pre-osteoblasts and/or osteoblasts.

In certain embodiments, for BTE and/or repair applications in particular, it is contemplated that patient-derived bone progenitor cells may be added to the scaffolds as described herein to promote repair and/or recovery.

In still another embodiment, there is provided herein a use of any of the scaffold biomaterial or scaffold biomaterials as described herein for BTE, for bone grafting, for repair or regeneration of bone, or any combination thereof. In yet another embodiment, there is provided herein a use of any of the scaffold biomaterial or scaffold biomaterials as described herein for any one or more of: craniofacial reconstructive surgery; dental and/or maxillofacial reconstructive surgery; major bone defect and/or trauma reconstruction; bone filler applications; implant stabilization; and/or drug delivery; or any combinations thereof. In still another embodiment, there is provided herein a use of any of the scaffold biomaterial or scaffold biomaterials as described herein in a dental bone filler application. In another embodiment, there is provided herein a use of any of the scaffold biomaterial or scaffold biomaterials as described herein as stress shielding reducers for large implants.

In yet another embodiment, there is provided herein a use of any of the scaffold biomaterial or scaffold biomaterials as described herein for promoting active osteogenesis; for implanting to repair critical and/or non-critical size defects; to provide mechanical support during bone repair; to substitute in loss or injury of long bones, calvarial bones, maxillofacial bones, dental, and/or jaw bones; for orthodontal and/or peri dental grafts, such as alveolar ridge augmentation, tooth loss, tooth implants and/or reconstructive surgery; for grafting at specific site(s) to augment bone volume due to loss from osteoporosis, bone loss due to age, previous implant, and/or injuries; or to improve bone-implant tissue integration; or any combinations thereof.

In yet another embodiment, there is provided herein a method for engineering bone tissue; for bone grafting; for repair or regeneration of bone; for craniofacial reconstructive surgery; for dental and/or maxillofacial reconstructive surgery; for major bone defect and/or trauma reconstruction; for dental or other bone filler application; for implant stabilization; for stress shielding of a large implant; for promoting active osteogenesis; for repairing critical and/or non-critical size defects; for provide mechanical support during bone repair; for substituting for loss or injury of long bones, calvarial bones, maxillofacial bones, dental, and/or jaw bones; for orthodontal and/or peri dental grafting such as alveolar ridge augmentation, tooth loss, tooth implants and/or reconstructive surgery; for grafting at a specific site to augment bone volume due to loss from osteoporosis, bone loss due to age, previous implant, and/or injuries; for improving bone-implant tissue integration; or for drug delivery; or for any combinations thereof; said method comprising:
providing a scaffold biomaterial as described herein; and
implanting the scaffold biomaterial into a subject in need thereof at a site or region in need thereof.

In certain embodiments, the scaffold biomaterial may be implanted at a site of injury (for example, a fracture, void filler, damaged bone tissue). In certain embodiments, scaffold biomaterials may be cell-free, or pre-seeded with cells which may, optionally, be from the patient (i.e. autologous) or from a donor (i.e. allogenic). In certain embodiments, scaffold biomaterials may be pre-formed, modular, or shaped in situ to match the defect or injury site. In certain embodiments, osteogenic growth factors may be pre-loaded into the scaffold biomaterials prior to implantation, or may be administered post implantation and/or post-op, or both.

In certain embodiments, such as for treating small breaks or cracks, wrapping or injecting of the scaffold biomaterial may be desirable. In certain embodiments, such as for larger defects, insertion of the scaffold biomaterial may be desirable.

In certain embodiments, scaffold biomaterials may be implanted as the site of a bone fracture or break, may be wrapped around bones or inserted into a break or gap, or both. In certain embodiments, bone cells may be pre-seeded into the scaffold biomaterials, or subsequently introduced into the scaffold biomaterials. In certain embodiments, an agent which triggers differentiation of pre-osteoblasts may be present in the scaffold biomaterials or introduced into the scaffold biomaterials. In certain embodiments, scaffold biomaterials for implantation may be configured such that they do not need to be removed, or they may be removed after a period of time, for example.

In certain embodiments, the method may further comprise a step of adding or seeding bone progenitor or bone or bone tissue cells into the scaffold biomaterial prior to implantation. In certain embodiments, the bone progenitor or bone or bone tissue cells may comprise patient-derived cells. In certain embodiments, the cells may comprise preosteoblasts, osteoblasts, differentiated bone and/or calvaria tissue cells, or any combination thereof.

In certain embodiments, it is contemplated that scaffold biomaterials as described herein may be derived from and/or comprise cellulose, hemicellulose, chitin, chitosan, pectin, lignin, or any combinations thereof.

Provided herein are scaffold biomaterials, and uses thereof for BTE. It is contemplated that in certain embodiments, scaffold biomaterials as described herein may be used to provide mineralized surfaces which may be modulated, with various molecular ratios selected to modulate bioactivity, osteoinduction and/or osteointegration as desired.

Scaffold biomaterials as described herein may benefit from the complex geometries, porosities, and/or structures derived from their naturally occurring plant sources. Such scaffold biomaterials, by virtue of their chemical compositions, may also be poorly or non-biodegradable in vivo, which may be beneficial in bone tissue engineering (BTE) applications.

In certain embodiments, the scaffold biomaterials described herein may be substantially or at least partly cellulose-based. Such cellulose scaffolds may beneficially be poorly biodegradable in vivo, and may beneficially be readily coatable and/or pre-mineralizable to provide pre-coated scaffold biomaterials with desirable BTE properties.

In certain embodiments, scaffold biomaterials and/or grafts as described herein may be pre-coated with different molecular ratios (by varying the number of incubation cycles, and/or concentration of reagents, for example), providing tunability. In certain embodiments, the plant tissue source from which the scaffold biomaterials/grafts are derived may be selected to suit the particular application. For example, in certain embodiments, the underlying porosity, and/or pore interconnectivity may be selected for recruitment and/or integration of cells within the scaffold biomaterial/graft. As many macro and microscopic architectures may be found in nature, many options are available and choosing an appropriate source may allow for optimizing the performance of the scaffold biomaterials/grafts for the particular application of interest. For example, in certain embodiments, a non-homogeneous, less porous, compact material may be less efficient or desirable than a homogeneous, porous scaffold with specific pore size and pore interconnectivity for certain applications, and therefor plant tissue source may be selected accordingly.

In certain embodiments, it is contemplated that the scaffold biomaterials/grafts as described herein may be modified to alter the surface chemistry so as to provide for better adhesion of the pre-coating. In certain embodiments, one or more functional groups may be added to the surface for better adhesion of the coating, for example. In certain embodiments, such approaches may be used to add drugs, hormones, metabolites, etc., to scaffold biomaterials as described herein. In certain embodiments, attractants and/or deterrents for certain cell types may be used, and/or local environment (biochemical and/or physics) may be altered to suit particular applications. In certain embodiments, distinct local spatial and/or temporal cues may be provided to cells.

In certain embodiments, it is contemplated that addition of collagen and/or growth factors and/or stem cells (or progenitor cells) and/or other structural or functional proteins may be performed to further adjust and/or tailor the scaffold biomaterials/grafts as described herein for a particular application of interest.

In certain embodiments, scaffold biomaterials/grafts as described herein may be for use in any one or more of: craniofacial reconstructive surgery; dental and/or maxillofacial reconstructive surgery; major bone defect and/or trauma reconstruction; bone filler applications; implant stabilization; and/or drug delivery. In certain embodiments, scaffold biomaterials/grafts as described herein may be for use in dental bone filler applications. In certain embodiments, it is contemplated that scaffold biomaterials/grafts as described herein may be for use as stress shielding reducers for large implants.

In certain embodiments, scaffold biomaterials may be treated for surface, or complete, mineralization of the scaffold biomaterial with stochiometric and/or calcium-deficient hydroxyapatite. In certain embodiments, time-dependent or independent surface mineralization with stochiometric and/or calcium-deficient hydroxyapatite may be performed. In certain embodiments, time-dependent or independent surface charge modification of the material may be performed. In certain embodiments, composite materials of different mechanical properties may be used to modulate stress shielding, (i.e. bone-material response, for example). In certain embodiments, stress shielding may be adjusted such that stiffness of the relevant in vivo environment is substantially matched (i.e. strong enough for function but not overly stiff), so as to avoid or reduce bone degradation in adjacent tissue such as surrounding bone tissue.

In another embodiment, there is provided herein a method for differentiating cartilage or bone precursor cells to become cartilage or bone tissue cells, said method comprising:
culturing the cartilage or bone precursor cells on any of the scaffold biomaterial or scaffold biomaterials as described herein in a differentiation media;
wherein the culturing includes exposing the cultured cells to an increased atmospheric pressure above ambient pressure at least once.

In another embodiment, there is provided herein a method for differentiating cartilage or bone precursor cells to become cartilage or bone tissue cells, said method comprising:
culturing the cartilage or bone precursor cells on any of the scaffold biomaterial or scaffold biomaterials as described herein in a differentiation media;
wherein the culturing includes at least one treatment period during which the cultured cells are exposed to an increased atmospheric pressure above ambient pressure for at least part of the treatment period, wherein the treatment period is at least about 10 minutes in duration and is performed at least once per week;

thereby differentiating the cartilage or bone precursor cells into cartilage or bone tissue cells.

In certain embodiments, the cartilage or bone precursor cells may comprise any one or more of Mesenchymal stem cells; Skeletal stem cells; Induced pluripotent stem cells; Preosteoblast cells; Preosteoclast cells; Osteo-chondro progenitor cells; Perichondral cells; Chondroblast cells; Chondrocyte cells; or Hypertrophic chondrocyte cells; or any combinations thereof.

In certain embodiments, the resultant cartilage or bone tissue cells may comprise fully differentiated cells, or cells that are further differentiated or more mature precursor cells as compared with the initial cartilage or bone precursor cells. Different levels of differentiation may be desired depending on the particular application. In certain embodiments, the resultant cartilage or bone tissue cells may comprise any one or more of Osteoblast cells; Bone lining cells; Osteocyte cells; Osteoclasts; Chondrocyte cells; or Hypertrophic chondrocyte cells; or any combinations thereof.

General principles of bone precursor cell differentiation are described in Rutkovskiy, A., Stensløkken, K. O., & Vaage, I. J. (2016). Osteoblast Differentiation at a Glance. Medical science monitor basic research, 22, 95-106. https://doi.org/10.12659/msmbr.901142, which is herein incorporated by reference in its entirety.

In certain embodiments, the differentiation media may comprise any suitable cell culture media suitable to allow for differentiating of the precursor cells to the desired cartilage or bone tissue cells. The skilled person having regard to the teachings herein will be aware of a variety of cell culture mediums or broths suitable for preparing differentiated cells of a desired type. In certain embodiments, the differentiation media may comprise an osteogenic medium, such as an osteogenic medium containing the following: Dulbecco's Modified Essential Medium Or Minimum Essential Medium α; Fetal bovine Serum; Penicillin-streptomycin; Dexamethasone; Ascorbic Acid; B-glycerophosphate or Inorganic Phosphate. In certain embodiments, the differentiation media may comprise a chondrogenic medium, such as a chondrogenic medium containing the following: Dulbecco's Modified Eagle's Medium, Fetal bovine Serum, Penicillin-streptomycin, Dexamethasone (e.g. Sigma), Ascorbate-2-phosphate, Sodium pyruvate, Transforming growth factor-beta 1 (TGF-β1, e.g. Peprotech, Rocky Hill, N.J.).

In certain embodiments, the increased atmospheric pressure may be any suitable atmospheric pressure that is above the ambient pressure. In certain embodiments, the ambient pressure may comprise a pressure of less than about 1 GPa. In certain embodiments, the increased atmospheric pressure may be selected to simulate a load normally placed on a bone tissue. In certain embodiments, the increased atmospheric pressure may be about 100 to about 1000 kPa above ambient pressure, such as about 200 to about 500 kPa, or about 250 to about 350 kPa, or any integer value within any of these ranges, or any subrange spanning between any two integer values within any of these ranges.

In certain embodiments, the treatment period may be at least about 10 minutes in duration, at least about 30 minutes in duration, at least about 1 hour in duration, or at least about 2 hours in duration, at least about 5 hours in duration, at least about 10 hours in duration, at least about 1 day in duration, at least about 2 days in duration, at least about 1 week in duration, or longer. In certain embodiments, the treatment period may be between about 10 minutes and about 2 weeks in duration, or any integer time value there between, or any subrange spanning between any two such integer time values.

In certain embodiments the treatment period may be performed at least once per week, at least twice per week, at least 3 times per week, at least 4 times per week, at least 5 times per week, at least 6 times per week, at least 7 times per week, at least 14 times per week, or more. In certain embodiments, the treatment period may be performed at a frequency of between once per week and 168 times per week, or any integer value therebetween, or any subrange spanning between any two such integer values. In certain embodiments, the treatment period may be performed at least once daily.

In yet another embodiment of any of the above method or methods, the cultured cells may be returned to a low or ambient pressure condition after each exposure to the increased atmospheric pressure. In certain embodiments, the cultured cells may be returned to a low pressure condition comprising a pressure which is lower than the increased atmospheric pressure, typically a low pressure that is close to ambient pressure. In certain embodiments, the cultured cells may be returned to an ambient pressure condition which is or is close to ambient pressure (typically about 101 kPa, for example).

In yet another embodiment of any of the above method or methods, the treatment period may comprise alternating the cultured cells between a low or ambient pressure condition, and an increased atmospheric pressure condition. In certain embodiments, the alternation may be slow, such that low/ambient and increased pressure phases are of longer duration, or the alternation may be fast such that low/ambient and increased pressure phases are short duration and alternate quickly. In certain embodiments, the transition from low/ambient pressure to increased pressure may be slow or fast. In certain embodiments, the transition from increased pressure to low/ambient pressure may be slow or fast. In certain embodiments, the rate of transition may be substantially linear, or may be non-linear.

In another embodiment of any of the above method or methods, the treatment period may comprise oscillating atmospheric pressure to which the cells are exposed between a low or ambient pressure and an increased atmospheric pressure. In yet another embodiment of any of the above method or methods, the treatment period may comprise oscillating atmospheric pressure to which the cells are exposed between a low or ambient pressure and an increased atmospheric pressure at a frequency of about 1-10 Hz, or any value there between, or any subrange therebetween.

In yet another embodiment of any of the above method or methods, the treatment period may comprise oscillating atmospheric pressure to which the cells are exposed between a low or ambient pressure and an increased atmospheric pressure, wherein the low or ambient pressure is ambient pressure (i.e. typically about 101 kPa+about 0 kPa) and the increased atmospheric pressure is about +280 kPa above ambient pressure (i.e. typically about 101 kPa+about 280 kPa=about 381 kPa), and optionally wherein the oscillating is at a frequency of about 1-10 Hz.

In still another embodiment of any of the above method or methods, the treatment period may comprise exposing the cultured cells to increased atmospheric pressure for a sustained duration. In yet another embodiment of any of the above method or methods, the treatment period may comprise exposing the cultured cells to a substantially constant increased atmospheric pressure for a sustained duration. In certain embodiments, the sustained duration may be at least about 10 minutes. In certain embodiments, the sustained duration may be about 10 minutes to about 3 weeks, or any time value therebetween, or any subrange therebetween.

In another embodiment of any of the above method or methods, the treatment period may be about 1 hour in duration, or longer.

In still another embodiment of any of the above method or methods, the treatment period may be performed once daily, or more than once daily.

In yet another embodiment of any of the above method or methods, the culturing may be performed for at least about 1 week.

In another embodiment of any of the above method or methods, the culturing may be performed for about 2 weeks, or longer.

In still another embodiment of any of the above method or methods, the increased atmospheric pressure may be applied as hydrostatic pressure.

In yet another embodiment of any of the above method or methods, the increased atmospheric pressure may be applied by modulating the pressure of a gas phase above the cultured cells.

In still another embodiment of any of the above method or methods, the increased atmospheric pressure may be about +280 kPa above ambient pressure (i.e. typically about 101 kPa+about 280 kPa=about 381 kPa).

### EXAMPLE 1

### Plant-Derived Biomaterials for Bone Tissue Engineering—Biomechanical Characterization of Cellulose Scaffolds for Bone Tissue Engineering In Vivo and In Vitro

Native macroscopic cellulose structures may be derived from various plants. It has been demonstrated that cellulose-based scaffolds derived from plants, using a surfactant treatment, may be used as a material for various tissue reconstructions by taking advantage of the native structure of the plant [14]. These biomaterials may be used for in vitro mammalian cell culture [14] and are biocompatible, and may become spontaneously vascularized subcutaneously [14]-[16]. Biomaterials may be sourced from specific plants according to the intended application [14]-[18]. For instance, vascular structure from plant stems and leaves display similar vascular structures to structures found in animal tissue [18]. Plant-derived cellulose scaffolds may also easily be carved into specific shapes and treated to alter their surface biochemistry [16]. A salt buffer may be included in the decellularization process, which may result in an increase in cell attachment, both in vitro and in vivo [16]. Plant-derived cellulose may be used in composite biomaterial by casting hydrogels onto the scaffold surface. Scaffolds may be biocompatible in animal, and may become spontaneously vascularized subcutaneously [15], [16]. Apple hypanthium tissue may provide a bone-like architecture, with interconnected pores ranging from 100 to 200 μm in diameter [14].

While other studies have shown promising results using bacterial cellulose for BTE [19], plant-derived cellulose has not been previously employed for this particular application in the present manner. Importantly, hypanthium tissue possesses a microstructure with geometric characteristics similar to trabecular bone [7]. In the following studies, it is demonstrated that apple-derived cellulose scaffolds may act as suitable biomaterial for BTE. Scaffolds derived from apple hypanthium tissue were prepared in two formulations via decellularization (see [14]-[16]).

In the following studies, MC3T3-E1 pre-osteoblast cells were seeded on bare cellulose scaffolds or composite scaffold biomaterials composed of a protein-based hydrogel (collagen hydrogel) embedded in cellulose scaffolds. Both scaffold preparations supported extensive cellular invasion and proliferation, at which point the scaffolds containing cells were placed in osteoinductive medium. After cell osteogenic differentiation, both scaffold types depicted a higher young's modulus, alkaline phosphatase activity, as well as calcium deposition and mineralization. Results support the suitability of low cost, sustainable, and renewable plant-derived scaffolds for BTE applications.

Naturally derived cellulose scaffolds may possess structural features of relevance to several tissues, support mammalian cell invasion and proliferation, as well as a high degree of in vivo biocompatibility. Decellularized apple hypanthium tissue may possess a pore size and properties similar to trabecular bone. As described herein, scaffolds as described herein may host osteoblastic differentiation. In this study, the potential of apple-derived cellulose scaffolds were examined as biomaterials for bone tissue engineering (BTE). The related mechanical properties in vitro and in vivo were also examined. To examine their in vitro mineralization potential, MC3T3-E1 pre-osteoblast cells were seeded on either bare cellulose scaffolds or on composite scaffolds composed of cellulose and collagen I. Following chemically induced differentiation, scaffolds were mechanically tested and evaluated for mineralization. The Young's moduli were found to increase after differentiation under both conditions. Alizarin Red and alkaline phosphatase staining further highlighted the osteogenic potential of the scaffolds and the mineralization on the scaffolds. Histological sectioning of the scaffold constructs reveal complete invasion by the cells and that mineralization occurred throughout the entire constructs. Finally, scanning electron microscopy and energy-dispersive spectroscopy demonstrated the presence of mineral aggregates deposited on the scaffolds after differentiation, and confirmed the presence of phosphate and calcium. Acellular scaffolds were implanted in rat calvarial defects and assessed for dislocation force and histology. Mechanical assessment revealed that dislocation force was of similar amount that native calvarial bone and other types of acellular implants. In summary, these results support that plant-derived cellulose may be employed for bone tissue engineering (BTE) applications.

Materials and Methods

Scaffold Preparation:

Samples were prepared following established methods [16]. Briefly, McIntosh apples (Canada Fancy) were cut in 8 mm-thick slices with a mandolin slider. The hypanthium tissue of the apple slices was cut into squares of 5 mm by 5 mm. Square tissues were decellularized in 0.1% sodium dodecyl sulfate (SDS, Fisher Scientific, Fair Lawn, N.J.) for two days. Decellularized samples were then washed in deionized water, followed by an overnight incubation in 100 mM in CaCl₂ to remove the remaining surfactant (see WO2017/136950, entitled “Decellularised Cell Wall Structures from Plants and Fungus and Use Thereof as Scaffold Materials”, herein incorporated by reference in its entirety, for further details). The samples were subsequently sterilized with 70% ethanol for 30 min, washed with deionized water, and placed in a 24-well culture plate prior to cell seeding. The scaffolds (8-mm thick) were either left untreated (bare scaffolds) or coated with a collagen gel solution (composite hydrogel scaffolds), as explained below.

Cell Culture and Scaffold Seeding:

MC3T3-E1 Subclone 4 cells (ATCC® CRL-2593™, Manassa, Va.) were used in this study, and were maintained at 37° C. in a humidified atmosphere of 95% air and 5% CO₂. The cells were cultured in Minimum Essential Medium (α-MEM, Gibco, ThermoFisher, Waltham, Mass.), supplemented with 10% Fetal Bovine Serum (FBS Hyclone Laboratories Inc., Logan, Utah) and 1% Penicillin/Streptomycin (Hyclone Laboratories Inc) and were allowed to grow to 80% confluency before being tryspinized. There were then resuspended resuspended at 10⁵ cells/mL in either α-MEM or a 1.5 g/L collagen solution, as follows, for the preparation of the bare scaffolds or the scaffolds coated with a collagen solution, respectively. Briefly, the collagen solution was prepared by mixing 50% (v/v) of 3 mg/mL type 1 collagen (ThermoFisher) with 2.5% of 1 N NaOH, 1% FBS, 10% of 10× phosphate-buffered saline (PBS, ThermoFisher), and 36.5% of sterile deionized water at 4° C. A 40 μL aliquot of cell suspension, in either α-MEM or a 1.5 g/L collagen solution, was pipetted on the scaffolds. The cells were left to adhere for 1 hour in cell culture conditions (i.e. at 37° C. in a humidified atmosphere of 95% air and 5% CO₂). Subsequently, 2 mL of culture medium was added to each culture well. Culture media was changed every 2-3 days, for 14 days. After these 14 days of incubation, differentiation of MC3T3-E1 was induced by adding 50 μg/mL of ascorbic acid and 4 mM sodium phosphate to the culture media (differentiation media). Differentiation medium was changed every 3-4 days for 4 weeks. Scaffolds in non-differentiation culture medium (without the supplements to induce differentiation) were incubated for the same period of time, with the same medium change frequency, and served as a negative control. All subsequent analyses were conducted at the end of this 4-week incubation period. Finally, the decellularized apple scaffolds as well as the cell-seeded bare and composite scaffolds were imaged after the 4-week incubation using a 12 megapixel digital camera.

Pore Size Measurements and Cell Distribution Analysis using Confocal Laser Scanning Microscopy:

To measure the scaffold pore size, decellularized apple scaffolds (prior to collagen treatment and MC3T3-E1 cell seeding) were thoroughly washed with PBS and stained with 1 mL of 10% (v/v) Calcofluor White solution (Sigma-Aldrich, St. Louis, Mo.) for 25 min in the dark and at room temperature. Subsequently, scaffolds (n=3) were washed with PBS and were imaged with a high-speed resonant confocal laser scanning microscope (Nikon Ti-E A1-R; Nikon, Mississauga, ON). ImageJ software [20] was used to process and analyze the confocal images. Briefly, maximum projections in the Z axis were created and the Find Edges function was used to highlight the edge of the pores. A total of 54 pores were analyzed (6 pores in 3 randomly selected area per scaffold, with n=3 scaffolds). Pores were manually traced using the freehand selection tool in ImageJ. The selections were fit as an ellipse to output the major axis length.

To analyze MC3T3-E1 cell distribution in the scaffolds, bare and composite cell-seeded scaffolds (n=3 for each experimental condition) were thoroughly washed with PBS and fixed with 4% paraformaldehyde for 10 min. They were then extensively washed with deionized water before permeabilizing the cells with a Triton-X 100 solution (ThermoFisher) for 5 min, and washed again with PBS. Staining of the scaffolds was carried out as previously described [14], [16]. Briefly, the scaffolds were incubated in 1% periodic acid (Sigma-Aldrich) for 40 min. After rinsing with deionized water, they were incubated in 100 mM sodium metabisulphite (Sigma-Aldrich) and 0.15 M hydrochloric acid (ThermoFisher), supplemented with 100 μg/mL propidium iodide (Invitrogen, Carlsbad, Calif.) for 2 h in the dark and at room temperature. Finally, they were washed in PBS, stained with 5 mg/mL DAPI (ThermoFisher) for 10 min in the dark, washed again, and stored in PBS prior to imaging. The cell-seeded surfaces of the scaffolds were imaged with a high-speed resonant confocal laser scanning microscope (Nikon Ti-E A1-R). ImageJ software [20] was used to process the confocal images and create a maximum projection in the Z axis for image analysis.

Young's Modulus Measurements:

Young's modulus measurements of the scaffolds (n=3 for each experimental condition) with non-differentiated and differentiated cells were obtained using a custom-built uniaxial compression apparatus. Decellularized apple-derived cellulose scaffolds without cells were used as a control. The force and position was recorded with a 150 g load cell (Honeywell) and an optical ruler. The force-displacement curves were obtained by compressing the samples at a constant rate of 3 mm min' and a maximum strain of 10%. The Young's modulus was obtained by fitting the linear portion of the stress-strain curve.

Alkaline Phosphatase and Alizarin Red S Staining:

Before staining with either 5-bromo-4-chloro-3′-indolyphosphate and nitro-blue tetrazolium (BCIP/NBT, ThermoFisher) or Alizarin Red S (ARS, Sigma-Aldrich), scaffolds were washed three times with PBS (without Ca²⁺ and Mg²⁺, Hyclone Laboratories Inc.) and fixed with 10% neutral buffered formalin for 30 min.

BCIP/NBT was used to assess the alkaline phosphatase (ALP) activity of cell-seeded scaffolds. BCIP/NBT staining solution was prepared by dissolving one BCIP/NBT tablet (Sigma-Aldrich) in 10 mL of deionized water. After fixation, the scaffolds (n=3 for each experimental condition) were washed with a 0.05% Tween solution and stained with BCIP/NBT for 20 min at room temperature. Finally, they were washed with 0.05% Tween and stored in PBS (without Ca²⁺ and Mg²⁺) prior to imaging.

ARS was used to assess calcium deposition and mineralization of the scaffolds. After fixation, the scaffolds (n=3 for each experimental condition) were washed with deionized water and exposed to 2% (w/v) ARS for 1 h at room temperature. They were then washed with deionized water to remove the excess ARS staining solution and stored in PBS (without Ca²⁺ and Mg²⁺) prior to imaging.

Finally, all scaffolds were imaged using a 12 megapixel digital camera.

Mineralization Analysis using Scanning Electron Microscopy and Energy-Dispersive Spectroscopy:

Scaffolds (n=3 for each experimental condition) were fixed in 4% para-formaldehyde for 48 h, followed by serial dehydration in increasing concentrations of ethanol (from 50% to 100%), as previously described [32]. Samples where then dried using a critical point dryer. Dried samples were gold-coated to a final coating thickness of 5 nm. Scanning electron microscopy (SEM) images were acquired with a JEOL JSM-7500F FESEM scanning electron microscope (JEOL, Peabody, Mass.) at 2 kV. Energy-dispersive spectroscopy (EDS) was performed on scaffolds seeded with MC3T3-E1 cells or non-seeded scaffolds. Three different areas of each scaffold surface were analyzed for mineral aggregates.

Rat Calvarial Defect Model

Bilateral craniotomy were performed following established protocol [33]. Male Sprague-Dawley rats (n=5) were anaesthetised with 3% isoflurane until unconscious and maintained under 2-3% isoflurane throughout the procedure. A 1.5 cm to expose the underlying cranium. Using a dental drill equipped with a 5 mm diameter trephine, defects were created in both parietal bones, on each side of the sagittal suture with constant irrigation of 0.9% NaCl. Surrounding bone was gently cleaned with 0.9% NaCl to remove any bone fragments. In this case, decellularized scaffolds were prepared exactly as above, however they were made into circular disks with a biopsy punch to match the 5 mm defect size. Control animals did not receive scaffolds. Overlying skin was closed with sutures. Rats were given unlimited access to food and water and were daily monitored by certified animal technicians at the Animal Care and Use Committee of the University of Ottawa. Rats were euthanized with CO₂ inhalation and thoracic perforation, as secondary euthanasia measure, after eight weeks post-implantation. Skin covering the skull was removed using a scalpel blade, exposing the cranium. Using a dental drill, the skull was cut at the frontal and occipital bones and side of both parietal bones, completely removing the top section of the skull. The samples were either placed in cold PBS and immediately assessed for mechanical assessment, or fixed with 10% formalin (Sigma-Aldrich, St. Louis, Mo.) for 72 hours. After fixation, the skulls were stored in 70% ethanol (Sigma-Aldrich, St. Louis, Mo.) and processed for histology.

Push-Out Test

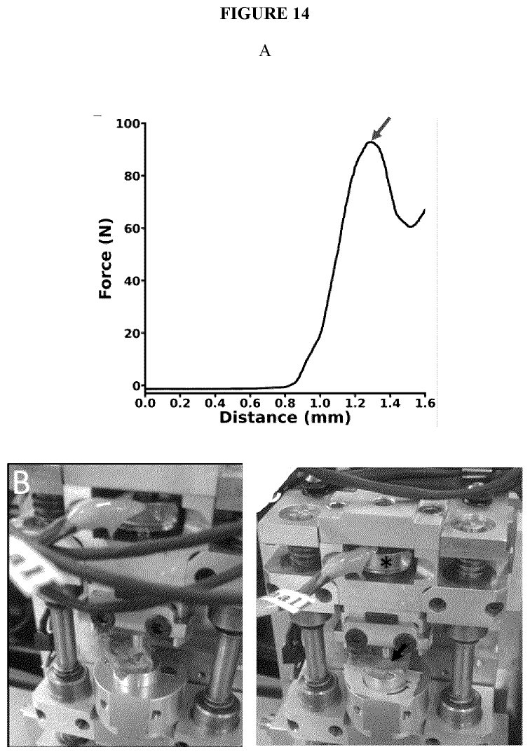

To assess the amount of force required to remove the implants from the surrounding bone, dislocation push-out tests were carried out after 8 weeks of implantation using a uniaxial compression device (MTI Instruments, Albany, N.Y.) and a 500 lbs load cell (Omega Engineering, Norwalk, Conn.)). After removal, the samples (n=7 implants; 4 animals) were placed with the dorsal side of the bone facing up on the sample holder (FIG. 14). The plunger was slowly lower until slightly touching one of the defects. The force vs distance curves were recorded until pass the full dislocation of the implants at 0.5 mm/min. Max force was recorded at break point in the force vs distance curve.

Histological Analysis:

In vitro scaffolds (n=1 for scaffolds in non-differentiation medium and n=2 for scaffolds in differentiation medium) were fixed in 4% para-formaldehyde for 48 h, and stored in 70% ethanol before paraffin embedding. Embedding, sectioning, and staining were performed by the PALM Histology Core Facility of the University of Ottawa. Briefly, 5 μm-thick serial sections were stained with hematoxylin and eosin (H&E; ThermoFisher) or Von Kossa (VK; ThermoFisher), starting 1 mm inside the scaffolds. Slides (n=2 per scaffold) were imaged using a Zeiss AXIOVERT 40 CFL microscope (Zeiss, Toronto, ON) to evaluate cell infiltration (H&E) and mineralization (VK). Image analysis was performed using ImageJ software. In vivo scaffolds were fixed as above, however all subsequent embedding, sectioning and staining was performed by AccelLAB Inc. (Boisbriand, QC). Embedded occurred in methyl methacrylate samples were serially cut in 6 μm sections, at three different levels, from the edge of the defects, towards the center of the implant. The sections contained both lateral defects. Sections were stained with either hematoxylin and eosin (H&E) or Goldner's Trichrome (GTC). Histological slides were imaged using a Zeiss AXIOVERT 40 CFL microscope to evaluate cell infiltration (H&E) and collagen deposition (MTC) of the implants. Images were analysed using ImageJ software.

Mineralization analysis using scanning electron microscopy (SEM) and energy-dispersive spectroscopy (EDS) Scaffolds (n=3 for each experimental condition) were fixed in 4% para-formaldehyde for 48 h, followed by serial dehydration in increasing concentrations of ethanol (from 50% to 100%), as previously described [21]. Samples where then dried using a critical point dryer. Dried samples were gold-coated to a final coating thickness of 5 nm. SEM images were acquired with a JEOL JSM-7500F FESEM scanning electron microscope (JEOL, Peabody, Mass.) at 2 kV. EDS was performed on bare scaffolds and composite hydrogel scaffolds seeded with MC3T3-E1 cells. Three different areas of each scaffold surface were analyzed for mineral aggregates.

Statistical Analysis:

All data are reported as mean±standard error of the mean (S.E.M.). The data were assumed to be normally distributed. Statistical analysis was performed using a one-way ANOVA followed by Tukey post-hoc tests for Young's moduli mean comparison. Student's T-test was performed for bone volume density comparison. A value of p<0.05 was considered to be statistically significant.

Results

The present studies investigated the mechanical properties of these scaffolds in vitro and in vivo. The present results show that scaffolds with differentiated osteoblasts had a Young's modulus of 193.8±16.4 kPa, which is much higher than scaffolds with non-differentiated cells (23.9±1.2 kPa) and acellular scaffolds (24.4±0.9 kPa). Moreover, after implantation for 8 weeks in a rodent calvarial defect model, cells are able to integrate the scaffolds into the surrounding bone, leading to a measured dislocation force of 114±18 N, similar to previous reports of cortical bone displacement [24].

Scaffold Imaging and Pore Size Measurements

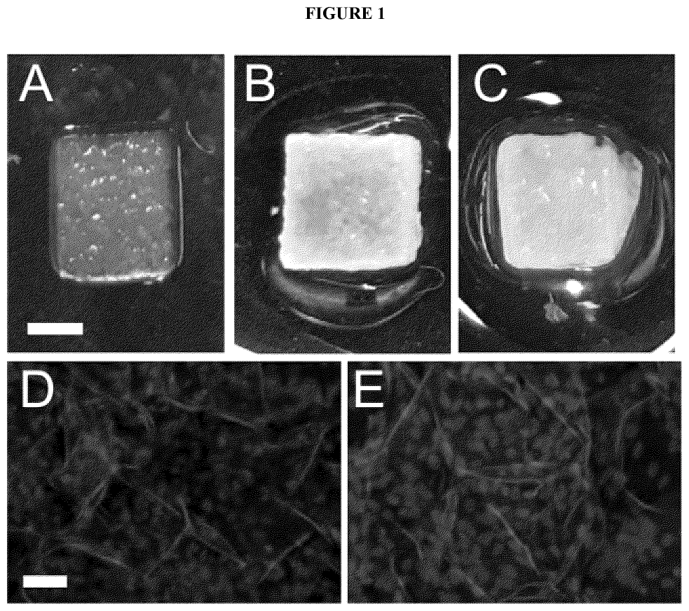

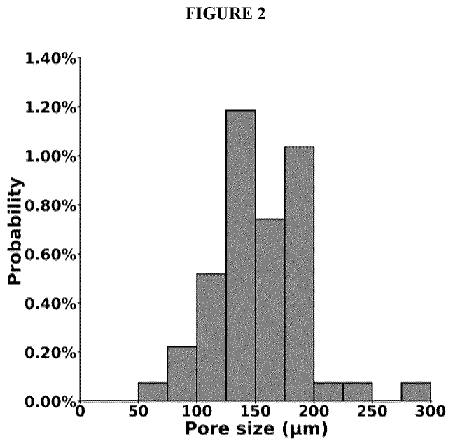

Complete removal of native cellular components of the apple tissue was achieved after SDS and CaCl₂ treatments (FIG. 1A, 1B, 1D). This process has been described in detail herein, and results in a three-dimensional (3D) scaffold that supports the infiltration and proliferation of many cell types. After seeding the scaffold with MC-3T3 pre-osteoblasts, they were grown to confluence and maintained in differentiation medium for up to four weeks (FIG. 2). At this point white mineral deposits were observed throughout the scaffolds as expected for successful differentiation of the cells. White calcium deposits were observed throughout the bare and composite hydrogel scaffolds cultured with differentiation medium for 4 weeks (FIGS. 1B and C, respectively). Both types of scaffolds with differentiated cells had a distinct opaque white colour that was absent in the control scaffolds without cells (FIG. 1A).

Confocal laser scanning microscopy showed that cells were homogeneously distributed in the bare scaffolds as well as the composite hydrogel scaffolds (FIGS. 1D and E, respectively, and 4B). The highly porous nature of the scaffolds is easily observed in the confocal images. Image quantification reveal that the decellularized apple-derived cellulose scaffolds (prior to collagen treatment and before MC3T3 cell seeding) displayed an average pore size of 154±40 μm. The pore size distribution ranged from 73 μm to 288 μm, with the majority of the pores being between 100 and 200 μm (FIG. 2).

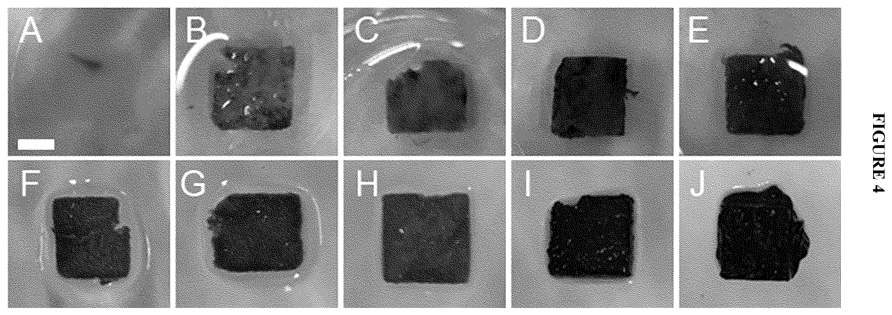

To analyze alkaline phosphatase (ALP) activity and mineralization, the scaffolds were stained with BCIP/NBT and ARS, respectively (FIGS. 4A-E and F-J, respectively). The BCIP/NBT staining results reveal that ALP activity increases significantly (as indicated by the strong purple colour) compared to scaffolds incubated in without cells, or with cells that were not maintained in differentiation media. Likewise, cells in scaffolds cultured in differentiation medium displayed a stronger red color after ARS staining indicating a higher degree of calcium mineralization than control scaffolds (no cells) or scaffolds with cells cultured in non-differentiation medium. However, some background staining is clearly visible in the controls and we speculate this may be due to the use of CaCl₂ in the decellularization protocol.

Mechanical Properties

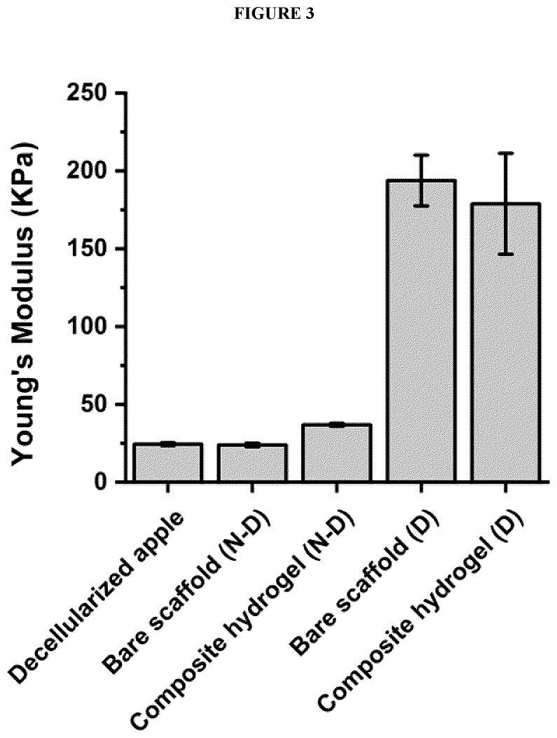

To investigate the mechanical properties of the scaffolds, the Young's modulus of the scaffolds was determined after being maintained in culture. The Young's moduli of both scaffold types (bare and composite hydrogel) as well as control scaffolds (without cells) were measured after the 4 weeks of incubation in either non-differentiation or differentiation medium (FIG. 3).

Results showed no significant difference in the Young's modulus between the control scaffolds (scaffolds without cells) (24.4±0.9 kPa) and the bare scaffolds as well as the composite hydrogel scaffolds cultured in non-differentiation medium (23.9±1.2 kPa p=0.9 and 36.9±1.0 kPa, respectively) (FIG. 3). On the other hand, a significant difference was observed between the control scaffolds (24.4±0.9 kPa) and the bare scaffolds as well as the composite hydrogel scaffolds cultured in differentiation medium (193.8±16.4 kPa and 178.9±32.4 kPa, respectively; p<0.001 in both cases). Furthermore, the Young's moduli of the scaffolds cultured in non-differentiation and differentiation media were significantly different for both the bare and the composite hydrogel scaffolds (p<0.001 in both cases). However, there was no significant difference between the Young's moduli of the bare and the composite hydrogel scaffolds cultured in either non-differentiation or differentiation medium. Alkaline phosphatase and Alizarin Red S staining To analyze ALP activity and mineralization, the scaffolds were stained with BCIP/NBT and ARS, respectively (FIG. 4).

BCIP/NBT staining (reflecting ALP activity) was much stronger in the bare scaffolds and the composite hydrogel scaffolds with differentiated cells (FIGS. 4D and E, respectively) than in the scaffolds (both types) with non-differentiated cells (FIGS. 4B and C, respectively). The control scaffolds (scaffolds without cells) did not show any staining (FIG. 4A). In addition, no difference in staining was observed between the bare scaffolds and the composite hydrogel scaffolds cultured in either non-differentiation (FIGS. 4B and C) or differentiation medium (FIGS. 4D and E).

Cells in both the bare and the composite hydrogel scaffolds cultured in differentiation medium displayed a stronger red color after ARS staining (FIG. 4I, J) than cells in the scaffolds (both types) cultured in non-differentiation medium (FIGS. 4G, H). Control scaffolds (without cells) and scaffolds with cells cultured in non-differentiation medium displayed a non-specific staining, but this coloration was much lighter (FIG. 4F-H).

Histology Analysis

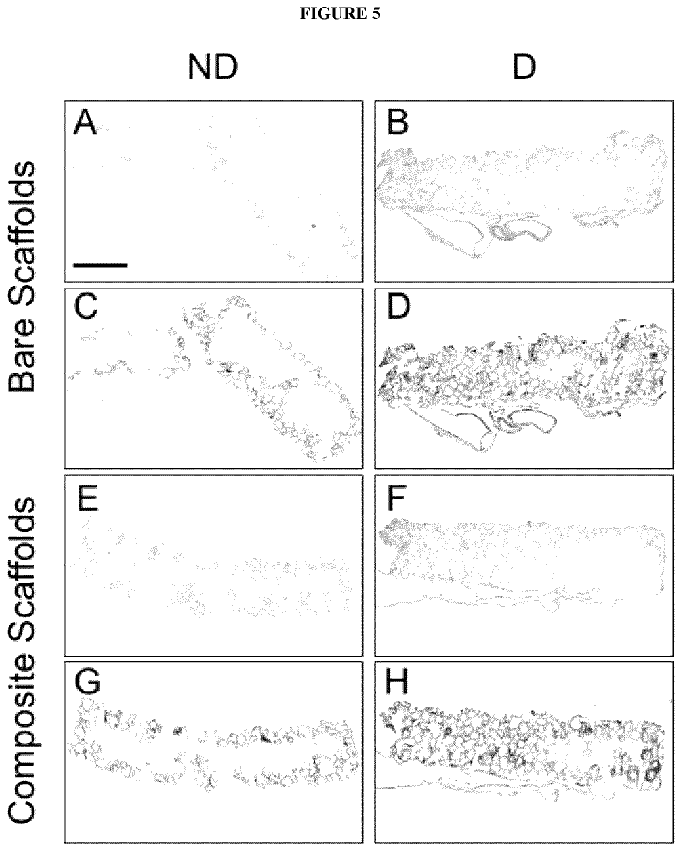

To further examine the contribution of CaCl₂ and osteoblasts in the deposition of calcium on the scaffold surfaces, histological staining, scanning electron microscopy (SEM) and energy-dispersive spectroscopy (EDS) were employed. Histological analysis was used to evaluate cell infiltration and scaffold mineralization. The scaffolds were fixed, embedded in paraffin, and stained with H&E or VK. Cell infiltration was demonstrated using H&E (FIG. 5A, B, E, F) and scaffold mineralization was analyzed using VK staining (FIG. 5C, D, G, H).

Bare scaffolds and composite hydrogel scaffolds were completely infiltrated with MC3T3-E1 cells (FIG. 5). Cell infiltration as demonstrated with H&E (FIG. 5) shows that both the non-differentiated and differentiated scaffolds displayed good infiltration with MC3T3-E1 cells. Multiple nuclei and cytoplasm were visible in the periphery and through the constructs (FIG. 5 A, B, E, F, blue and pink, respectively). Collagen was also visible in pale pink and more pronounced in the composite hydrogel scaffolds. The pore walls in the bare scaffolds and composite hydrogel scaffolds were entirely stained in black after the 4-weeks of culture in differentiation medium (FIGS. 5G and H, respectively). The pore walls of the bare scaffolds and the composite hydrogel scaffolds cultured in non-differentiation medium only showed the presence of mineralization on the outside periphery of the constructs (FIGS. 5C and D, respectively).

Mineralization analysis using scanning electron microscopy and energy-dispersive spectroscopy Samples were fixed and imaged using SEM for mineral aggregates. EDS was performed to analyze the chemical composition of the aggregates.

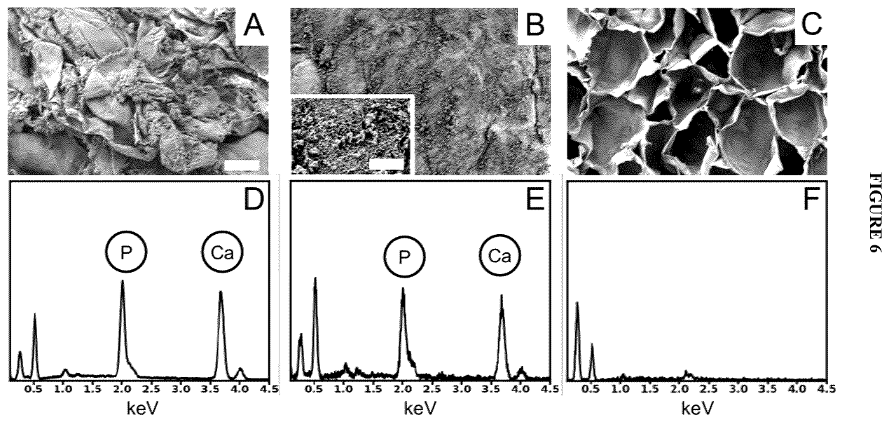

Localized mineralization was visible in the bare scaffolds and the composite hydrogel scaffolds seeded with cells after 4 weeks of culture in differentiation medium (FIGS. 6A and B, respectively). Mineral deposits appeared as globular aggregates on the edge of the pores for both types of scaffolds. No mineral aggregates were visible on the bare scaffolds without cells (FIG. 6C). EDS spectra were acquired on selected regions of interest, namely on the mineral aggerates for the cell-seeded scaffolds (FIGS. 6D and E) and on pore walls for the non-seeded scaffolds used as a control (FIG. 6F). The spectra displayed stronger signal of phosphorous (P) and calcium (Ca) in both types of scaffolds cultured in differentiation medium, compared to the non-seeded scaffolds.

VK staining revealed that the pore walls of the scaffolds were entirely stained in black after the 4-weeks of culture in differentiation medium. The pore walls of the scaffolds cultured in non-differentiation medium only showed the presence of mineralization on the outside periphery of the constructs and it is contemplated (without wishing to be bound by theory) that this may be largely due to the absorption of calcium from the decellularization treatments. Samples were also fixed and imaged using SEM to analyze the chemical composition the mineral deposits on the undifferentiated and differentiated scaffolds (FIGS. 6A and D showing Mineralized, FIGS. 6C and F showing Control). Localized mineralization was visible in the scaffolds seeded with cells after 4 weeks of culture in differentiation medium. Mineral deposits appeared as globular aggregates on the edge of the pores. No mineral aggregates were visible on control scaffolds. EDS spectra were acquired on selected regions of interest, namely on the mineral aggerates for the cell-seeded scaffolds (FIG. 6) and on pore walls of the controls control. The spectra clearly displayed distinct characteristic signals corresponding to the deposition of phosphorous (P) and calcium (Ca) in scaffolds cultured in differentiation medium, compared to the non-seeded scaffolds.

Discussion

Plant-derived cellulose biomaterials have potential in various fields of regenerative medicine. In vitro and in vivo studies have shown the biocompatibility of plant-derived cellulose and their potential use for tissue engineering [14]-[18]. An aim of the presently described study (and that of Example 4) was to investigate the potential of plant-derived cellulose to be used as a material for BTE using two approaches: in vitro and in vivo. This was accomplished by further investigating the change in Young' s moduli of the scaffolds in vitro and measuring the dislocation force of the implants in vivo. The present studies support plant-derived scaffold biomaterials for use in BTE.

After removing the native cells from the apple tissue, pre-osteoblast cells (MC3T3-E1) were seeded in either bare scaffolds or composite hydrogel scaffolds (scaffolds coated with a collagen solution). The cells were let to proliferate and infiltrate the scaffold constructs for 14 days before inducing osteogenic differentiation by using differentiation medium for 4 weeks (scaffolds cultured in non-differentiation medium served as a control).

Using confocal microscopy, compression measurements, mineralization staining, histology, SEM and EDS, these studies show that the cells were able to proliferate and differentiate within the scaffolds, thereby supporting the use of plant-derived cellulose scaffolds to support bone formation. Confocal laser scanning microscopy confirmed that the cells adhered to the bare cellulose scaffolds and the composite hydrogel scaffolds (FIGS. 1D and E, respectively). Interestingly, calcium deposits were observed in the scaffolds (FIGS. 1B and C), and more specifically on the edge of the pores. The shape (globular) of these aggregates for both types of scaffolds was noted. In addition, a large number of cell nuclei was observed around the cellulose pores as well as inside the scaffold pores (FIGS. 1D and E). Moreover, it was observed that the diameter of the scaffold individual pores was about 154 μm, with the majority of the pores being between 100 and 200 μm (FIG. 2). This is in line with the optimum pore size for bone growth, which has been shown to be in the range of 100-200 μm [7].

Furthermore, a significant change (about 3 to 8-fold increase) in the Young's modulus of both the bare scaffolds and the composite hydrogel scaffolds was demonstrated after culture in differentiation medium (FIG. 3, 5, 6). On the other hand, the addition of cells in the bare or the composite hydrogel scaffolds cultured in non-differentiation medium did not significantly affect the Young's modulus of the constructs, and the modulus was similar to that of the control scaffolds (without cells). Interestingly, no significant differences were observed between the bare scaffolds and the composite hydrogel scaffolds cultured in either non-differentiation or differentiation medium. Overall, these results indicate that the mineralization in either type of scaffolds cultured in differentiation medium resulted in an increase of the Young's modulus, but the presence of type 1 collagen gel in the composite scaffolds did not further increase the Young's modulus. It should be noted that despite the increase in the Young's modulus of both types of scaffolds when cultured in differentiation medium, the moduli was lower than that of bone (0.1 to 2 GPa for trabecular bone and 15 to 20 GPa for cortical bone [8]) and so the particular scaffolds of this example may be more desirable for non-load bearing applications (e.g., fractures in hand and wrist) as compared with load-bearing applications.

Staining results revealed a higher expression of ALP (FIGS. 4D and E) and the presence of more calcium deposits (FIGS. 4D and E) within both types of scaffolds after 4 weeks of culture in differentiation medium (FIGS. 4I and J) than in the control scaffolds (FIGS. 4A and F) and in both types of scaffolds cultured in non-differentiation medium (FIGS. 4B, C and G, H, respectively). Histological analysis showed invasion and proliferation of MC3T3-E1 cells in both types of scaffolds (FIG. 5A, B, E, F), with also a similar cell distribution. The pore walls of the constructs were mineralized by the osteoblasts after the 4-week differentiation period (FIGS. 5D and H) in both types of scaffolds. Of note is that the periphery of the constructs with non-differentiated cells was also stained with VK. This non-specific staining may have been due to residual CaCl₂ in the scaffolds after the decellularization process. Visual confirmation of mineralization was further assessed by qualitative analyses of SEM pictures. After the 4-week period in differentiation medium, both cell-seeded scaffold types displayed signs of ECM mineralization. Indeed, aggregates of minerals were visible on the scaffold constructs, specifically on the edges of the pores (FIG. 6), which agrees with a study by Addison et al. [35] using MC3T3-E1 extracellular matrix. These aggregates were not visible on the bare scaffolds without cells. EDS analysis of the aggregates revealed high level of P and Ca, thereby suggesting the presence of apatite on the scaffold constructs.

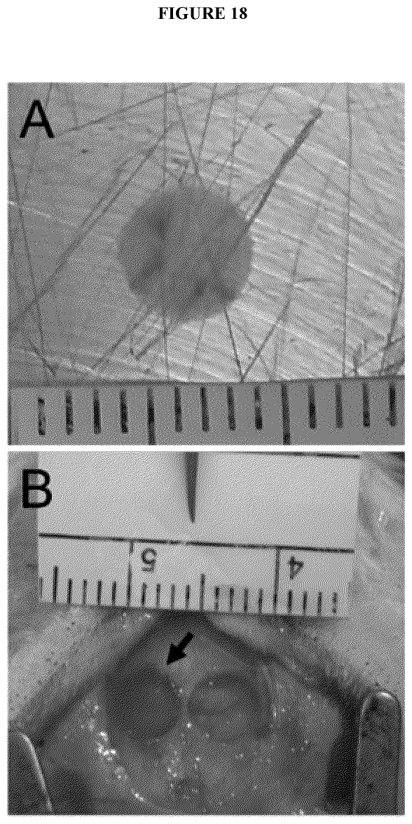

Decellularized apple scaffolds were implanted in 5 mm critical-sized cranial defects in rats. Implants were removed after 8 weeks for mechanical assessment or to be processed for histology. Mechanical assessment of the dislocation force indicated an average value of 114±18 N. The amount of force required to dislocate the implants from the surrounding bone is similar to the amount of force required to displaced intact calvarial bone (FIG. 14A), as reported by Zhao et al., 2012 (127.06±9.58 N) [36]. Thus, indicating that the implants are attached to the surrounding bone and connective tissues. Moreover, the dislocation force is similar to what has been reported after 8 weeks implantation using calcium-deficient hydroxyapatite scaffolds loaded with bone-morphogenic protein 2 (119.12±17.82 N) [36]. Histological analysis revealed the presence of cells within the scaffolds and punctured canals (FIGS. 14, 18), at 4 and 8 weeks revealed by H&E staining. Blood vessels were also visible within the scaffolds (FIGS. 14, 18). Furthermore, type 1 collagen was observed within the scaffold at 4 and 8 weeks by MTC staining.

In these studies it is demonstrated that pre-osteoblast cells can adhere and proliferate within apple-derived cellulose scaffold constructs, either untreated or coated with a collagen solution. Mineralization occurred within both types of scaffolds after chemically inducing osteogenic differentiation of pre-seeded pre-osteoblasts, which resulted in an increase in the Young's modulus of the constructs. Interestingly, these apple-derived scaffolds had a suitable pore size for BTE applications. Implanted plant-derived cellulose scaffolds required similar amount of force to be dislocated from the implant site as calvarial bone and other type of scaffolds used for BTE. Cells infiltrated the implant and deposited type 1 collagen. Overall, results support plant-derived cellulose as biomaterial for BTE applications.

### EXAMPLE 2

### Plant-Derived Biomaterials Pre-Coated (Pre-Mineralized) with Apatite for Bone Tissue Engineering

Custom three-dimensional scaffolds, matrices, grafts and/or artificial tissues for bone tissue engineering applications are desirable. To construct such material, the native source (i.e. plant) was decellularized and features of interest (porous structures, micro and macro channels, semi-permeable membrane) were extracted and subsequently pre-coated with alternate solution of Calcium Chloride and Disodium phosphate.

When bone tissues are severely damaged, either by traumatic injury or various diseases, a graft or bone substitute may be desirable. Such bone graft may promote active osteogenesis. It may be implanted to repair critical and/or non-critical size defects. Such bone graft may provide mechanical support during bone repair. For example, such graft can be used to substitute in loss or injury of long bones, calvarial bones, maxillofacial bones, dental, and/or jaw bones. Such grafts may also be used for orthodontal and peri dental grafts, such as alveolar ridge augmentation, tooth loss, tooth implants and/or reconstructive surgery. It may also be grafted at specific site(s) to augment bone volume due to osteoporosis, bone loss due to age, previous implant, and/or injuries. Such graft may also be used to improve bone-implant tissue integration, for example.

For fabricating the grafts/scaffold biomaterials of these studies, apples were cut into slices (size and thickness depending on the size of the desired graft). Samples were carved, shaped, and extracted from apple slices. Then, samples were washed with phosphate buffered solution (PBS) and were decellularized with a 0.1% SDS solution, under agitation at room temperature for 48 hours. Furthermore, the samples were thoroughly washed with distilled water and were submerged in a 100 mM calcium chloride solution, under agitation at room temperature for 24 hours. Samples were thoroughly washed with distilled water and were sterilized with a 70% ethanol solution for 1 h, before being thoroughly washed with distilled water. Finally, the samples were stored in either 0.9% irrigation saline or sterile PBS at 4C until coating. See WO2017/136950, entitled “Decellularised Cell Wall Structures from Plants and Fungus and Use Thereof as Scaffold Materials”, herein incorporated by reference in its entirety, for further details on decellularization.

To coat the grafts, the grafts were submerged in a sterile 50 mM calcium chloride solution, under agitation at room temperature for 24 hours. The grafts were gently washed with sterile distilled water and submerged in a sterile 100 mM disodium phosphate, under agitation at room temperature for 24 hours. The grafts were gently washed with sterile distilled water and the alternating immersion cycle of calcium chloride-disodium phosphate was repeated until the desired thickness of the graft was achieved (thickness was visually assessed, see FIG. 6). The grafts were stored in either 0.9% irrigation saline or sterile PBS at 4C until use.

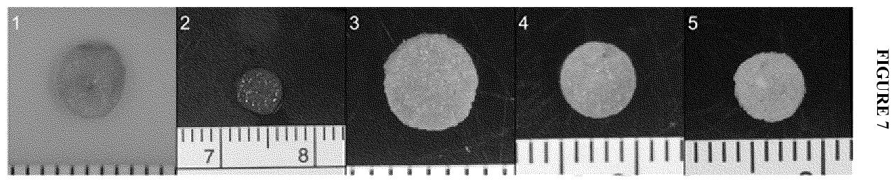

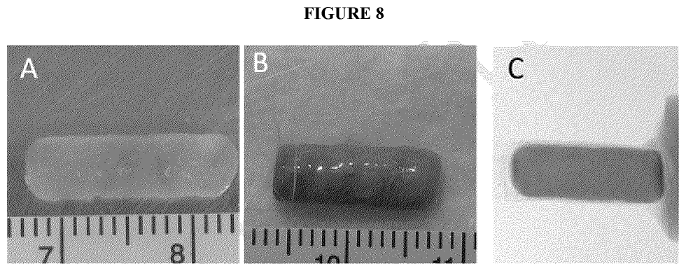

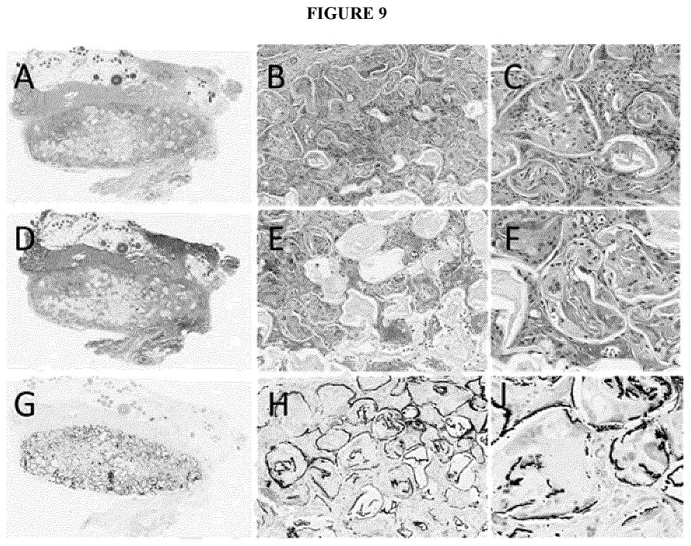

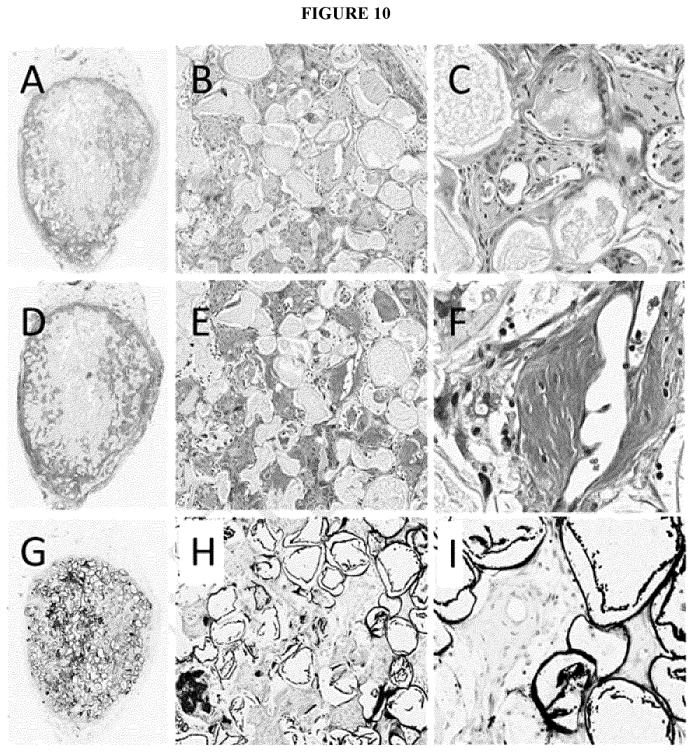

Two graft shapes were created: 5 mm by 1.5 cm cylinder and 5 mm by 1 mm disk (see FIGS. 7 and 8, and accompanying Figure legends). Both shapes were subcutaneously implanted in rat (N=1 per shape) in three different regions. The implants were retrieved after 4 weeks and processed for histological slicing and staining. Histological staining is shown in FIGS. 9 and 10.

FIG. 7 shows time-evolution of the coating. FIG. 8 shows rod-shaped material before implantation, after implantation, and x-ray after implantation. FIG. 9 shows histological staining of disk-shaped material after implantation. FIG. 10 shows histological staining of rod-shaped material after implantation.

### EXAMPLE 3

### Composite Biomaterials

In these studies, it was sought to develop composite biomaterials, combining 2 or more scaffold biomaterials and/or grafts as described herein, so as to provide even further tunability to scaffold biomaterials and/or grafts as described herein. As will be understood, such composite biomaterials may be desirable not only in BTE applications as described herein, but also in a wide variety of other applications in which scaffold biomaterials may be used, and adjustability of scaffold structural and/or chemical properties is desirable.

In this study, different biomaterial subunits were combined via gluing. Although many glues may be possible, this study used gelatin crosslinked with glutaraldehyde glue (reduced with sodium borohydride). First, the starting material was carved into its desired shape. The desired shape was then removed from the bulk material by slicing on a Mandolin slicer. The thickness of the Mandolin slice sets the z thickness of the material. Subsequently, the material was decellularized and sterilized as per the examples above and WO2017/136950, entitled “Decellularised Cell Wall Structures from Plants and Fungus and Use Thereof as Scaffold Materials”, herein incorporated by reference in its entirety.

The material was then ready for cell culture/implantation and was readily assembled into the final unit by gluing. The glue rapidly solidified and was stronger than fibrin glue. The strength may be modified by adjusting the ratio of the gelatin and gluteraldhyde. The gelatin was prepared by autoclaving the gelatin powder in media or water. It was then heated to 37° C. and the glutaraldehyde was introduced (a typical ratio consist of 1 mL of 10% gelatin with 5 μL of glutaraldehyde). The solution was mixed quickly, and then pipetted onto the adhesion site.

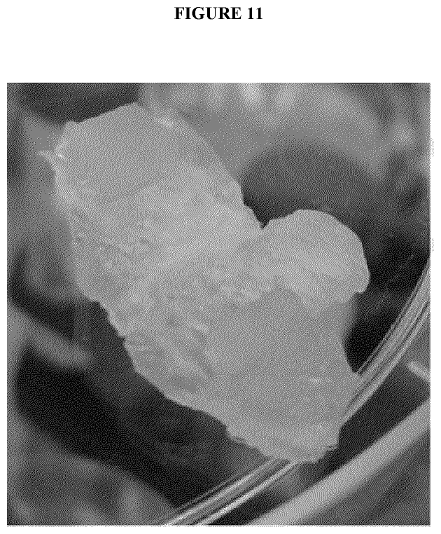

FIG. 11 shows an image of a hanging membrane (decellularized orange pith) glued and sandwiched between decellularized apple hypathium tissue, prepared as described above.

Results indicate that gluing in such manner may provide benefit in terms of overcoming size limitations of starting materials by assembling two or more subunits to provide a larger size; overcoming lengthy decellularization of large materials by using smaller materials and then assembling together; overcoming diffusion difficulties of large constructs; allowing for designing of certain structures and/or features that are not normally found in nature while exploiting the natural complexity of the scaffold biomaterial in the individual subunits; allowing for more complicated physical and/or mechanical properties to be produced (i.e. stress shielding and/or site specific moduli, channels, pores, etc.); and/or allowing for the combination of different cell types in different regions; or any combinations thereof.

In certain embodiments, it is contemplated that approaches as described herein may be amenable to a variety of modifications such as gluing, gel casting, chemical functionalization, loading (i.e. drugs, signalling molecules, growth factors, metabolites, etc.), any or all of which may vastly expand and/or provide adjustability of functionality of the materials.

In certain embodiments, it is contemplated that the approaches herein may allow for drugs, signalling molecules, growth factors, metabolites, ECM proteins, and/or other components to be added to the scaffold biomaterials and/or grafts as modifications. In certain embodiments, the approaches herein may allow for customization in terms of hydrogel casting, gluing, chemical modifications, and/or crosslinking, for example.

In certain embodiments, it is contemplated that scaffold biomaterials as described herein may be derived from and/or comprise cellulose, hemicellulose, chitin, chitosan, pectin, lignin, or any combinations thereof. In certain embodiments, it is contemplated that biochemical, biophysical, and/or mechanical properties of cellulose, hemicellulose, chitin, chitosan, pectin, and/or lignan scaffolds may be tunable.

In certain embodiments, it is contemplated that time dependent/independent release of drugs, signalling molecules, growth factors, metabolites, ECM proteins and/or other components may be provided by scaffold biomaterials and/or grafts as described herein.

In certain embodiments, it is contemplated that shapes and/or structures of scaffold biomaterials and/or grafts as described herein may be customizable through composites, glues, coatings, gels, and/or pastes selection and/or manipulation.

In certain embodiments, it is contemplated that large macro objects may be created with varying degrees of flexibility and/or articulation. In certain embodiments, it is contemplated that two or more subunits may be combined to make larger macrostructures, for example. In certain embodiments, it is contemplated that geometry may be used to hold subunits together, rather than, or in addition to, glue. In certain embodiments, it is contemplated that such approaches may be of use for bone tissue engineering due to the different structures which may be involved (for example, spongy versus cortical bone, etc. . . . ).

In certain embodiments, it is contemplated that the present composite materials and gluing methods may be for use in any one or more of: custom in vitro 3D cell culture devices; in vivo research; medical devices; bone, connective tissue, skin, muscle, nerve and/or interfaces; complex tissue repair and/or replacements; membranes and/or filters (i.e. artificial kidneys and/or simple biochemistry separation columns); vectors for site specific and/or time specific drug delivery; increased biocompatiblilty of existing medical devices through coating or creating composites with the present scaffold biomaterials; vectors for primary cell culture; cosmetic procedures (i.e. implants, and/or subdermal topographies); stents and/or shunts; non medical applications such as articulating parts for synthetic biorobotics; electrical circuitry integration; or any combinations thereof. In certain embodiments, it is contemplated that the present composite materials and gluing methods may be for use in complex tissue design and/or biomaterial implants for tissue repair/regeneration.

### EXAMPLE 4

### In Vivo Critical Size Calvarial Defect Model

The present study was conducted to evaluate the potential of scaffold biomaterials as described herein for bone regeneration applications, in a rat critical-size, bilateral defect model. The biomaterials (non-treated) were implanted in rats for periods of 4 and 8 weeks. 5 mm bilateral, circular defects were created on rats calvarium. Once the bone defects were excised, the biomaterials (5 mm diameter by 1 mm thickness) were placed within the defect. Overlying skin was sutured, and the rats were let to recovers for a period of 4 to 8 weeks. Specimens were collected at each time points and computational tomography (CT scan), implant dislocation mechanical testing and histology were performed.

This study was performed in 2 waves. In the first one (from CD0 to CD20), both apple and carrot where used as a source of decellularized plant tissue, however only the apple source implant were further tested for histology, CT scans and implant dislocation (see below). In the second wave (from 4WCH1 to 8WME3, see Label section), apple sourced biomaterials were perforated with a 200 μm needle, separated by 500 μm, in a grid-like pattern, to enhance diffusion and cell migration throughout the scaffold. Data shown here is obtained from animals in wave #2.

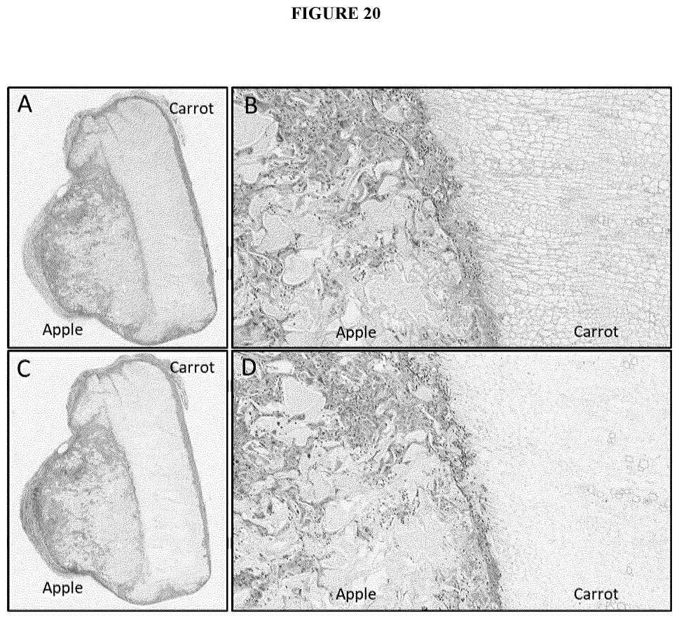

Due to the possible lack or reduction of cell infiltration of the carrot source under the conditions tested, carrot was not chosen as an optimal candidate in the present bone-related application. As shown in FIG. 20, cell invasion was rather poor when compared to the apple counter-part (interlocked composite of apples and carrot (SSC), implanted subcutaneously in rat for a 4-week period). It seems that the microstructure of the scaffold (pore size, pore interconnectivity and pore geometry) might plays a role in cell infiltration, with apple having more favourable characteristics. For instance, apple hypanthium tissue may provide a micro-architecture that resemble trabecular bone. Therefore, tissues with similar architecture may be excellent candidates for bone regeneration applications. Namely, plant-derived scaffolds with interconnected pores and pore sizes in the approximate range of about 100-200 μm may be optimal for such applications.

Labels:

| | | | | |
|---|---|---|---|---|
| Rat ID | Long name | Implant source | Time point | Comments |

Wave #1

| | | | | |
|---|---|---|---|---|
| CD0 | Non-perforated | Carrot | 8 Weeks | |
| | implant, Rat #0 | | | |
| CD1 | Non-perforated | Apple | 8 Weeks | Was processed in histology |
| | implant, Rat #1 | | | |
| CD2 | Non-perforated | Apple | 8 Weeks | |
| | implant, Rat #2 | | | |
| CD3 | Non-perforated | | | Non-survival |
| | implant, Rat #3 | | | |
| CD4 | Non-perforated | Apple | 8 Weeks | |
| | implant, Rat #4 | | | |
| CD5 | Non-perforated | Apple | 4 Weeks | |
| | implant, Rat #5 | | | |
| CD6 | Non-perforated | Apple | 8 Weeks | |
| | implant, Rat #6 | | | |
| CD7 | Non-perforated | Apple | 8 Weeks | |
| | implant, Rat #7 | | | |
| CD8 | Non-perforated | Apple | 4 Weeks | |
| | implant, Rat #8 | | | |
| CD9 | Non-perforated | | | Non-survival |
| | implant, Rat #9 | | | |
| CD10 | Non-perforated | Carrot | 8 Weeks | |
| | implant, Rat #10 | | | |
| CD11 | Non-perforated | Carrot | 8 Weeks | |
| | implant, Rat #11 | | | |
| CD12 | Non-perforated | Carrot | 8 Weeks | |
| | implant, Rat #12 | | | |
| CD13 | Non-perforated | Carrot | 8 Weeks | |
| | implant, Rat #13 | | | |
| CD14 | Non-perforated | | | Non-survival |
| | implant, Rat #14 | | | |
| CD15 | Non-perforated | Carrot | 4 Weeks | |
| | implant, Rat #15 | | | |
| CD16 | Non-perforated | Carrot | 4 Weeks | |
| | implant, Rat #16 | | | |
| CD17 | Non-perforated | Apple | 4 Weeks | |
| | implant, Rat #17 | | | |
| CD18 | Non-perforated | Apple | 4 Weeks | |
| | implant, Rat #18 | | | |
| CD19 | Non-perforated | Apple | 4 Weeks | |
| | implant, Rat #19 | | | |
| CD20 | Non-perforated | Carrot | 8 Weeks | |
| | implant, Rat #20 | | | |

Wave #2

| | | | | |
|---|---|---|---|---|
| 4WCH1 | Perforated implant, | Apple | 4 Weeks | Was imaged and used for CT scan |
| | Rat #1 | | | data. |
| 4WCH2 | Perforated implant, | Apple | 4 Weeks | Was processed in histology. Was |
| | Rat #2 | | | imaged and used for CT scan data. |
| 4WCH3 | Perforated implant, | Apple | 4 Weeks | Was processed in histology. Was |
| | Rat #3 | | | imaged and used for CT scan data. |
| 4WCH4 | Perforated implant, | Apple | 4 Weeks | Was imaged and used for CT scan |
| | Rat #4 | | | data. |
| 8WCH1 | Perforated implant, | Apple | 8 Weeks | Was processed in histology. Was |
| | Rat #5 | | | imaged and used for CT scan data. |
| 8WCH2 | Perforated implant, | Apple | 8 Weeks | Was imaged and used for CT scan |
| | Rat #6 | | | data. |
| 8WCH3 | Perforated implant, | Apple | 8 Weeks | Was imaged and used for CT scan |
| | Rat #7 | | | data. |
| 8WCH4 | Perforated implant, | Apple | 8 Weeks | Was processed in histology |
| | Rat #8 | | | Calvarial bone not completely |
| | | | | removed in both defects. (Not |
| | | | | counted in CT scan volume data). |
| | | | | Was imaged with CT scan. |
| 4WME1 | Perforated implant, | Apple | 8 Weeks | Was updated to an 8-week time |
| | Rat #9 | | | point. Left and right implant used for |
| | | | | push-out mechanical data. |
| 4WME2 | Perforated implant, | Apple | 8 Weeks | Was updated to an 8-week time |
| | Rat #10 | | | point. Upon removing the calvarium, |
| | | | | a small portion of the sagittal suture |
| | | | | split, close to the bregma. In push-out |
| | | | | test, poor connection between |
| | | | | implant and surrounding bone was |
| | | | | detected in the right implant. Not |
| | | | | used for mechanical data. |
| 8WME1 | Perforated implant, | Apple | 8 Weeks | Left and right implant used for push- |
| | Rat #11 | | | out mechanical data. |
| 8WME2 | Perforated implant, | Apple | 8 Weeks | Left and right implant used for push- |
| | Rat #12 | | | out mechanical data. |
| 8WME3 | Perforated implant, | Apple | 8 Weeks | When retrieving the implant in the |
| | Rat #13 | | | right defect, the implant had shifted |
| | | | | from the original defect position and |
| | | | | was not integrated to the |
| | | | | surrounding bone. Left implant used |
| | | | | for push-out mechanical data. |

Results:

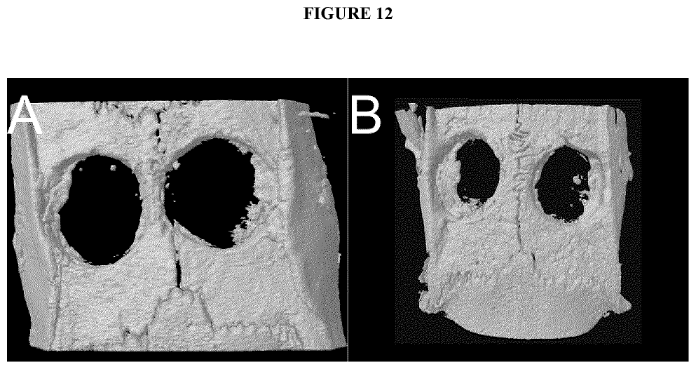

FIG. 12 shows three-dimensional rendering of implanted (biomaterial with perforations) critical size defects at 4 weeks (A) and 8 weeks (B).

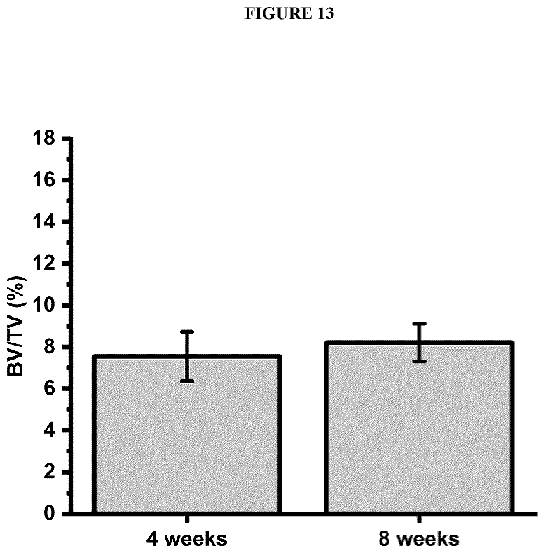

FIG. 13 shows bone volume fraction over total volume inside the defect. The Cylindrical volumetric ROI were obtained by fitting a cylinder with approximatively the same dimensions as the defect, in CT scan slices. N=6 defects (3 animals) for the 4 weeks-time point and N=6 defects (3 animals) for the 8 weeks-time point.

FIG. 14 shows a dislocation experiment. Typical force vs distance curve is shown in (A). The dislocation is taken as the approximative maximum force in the force vs distance graph (red arrow). Push-out device with specimen is shown in (B).

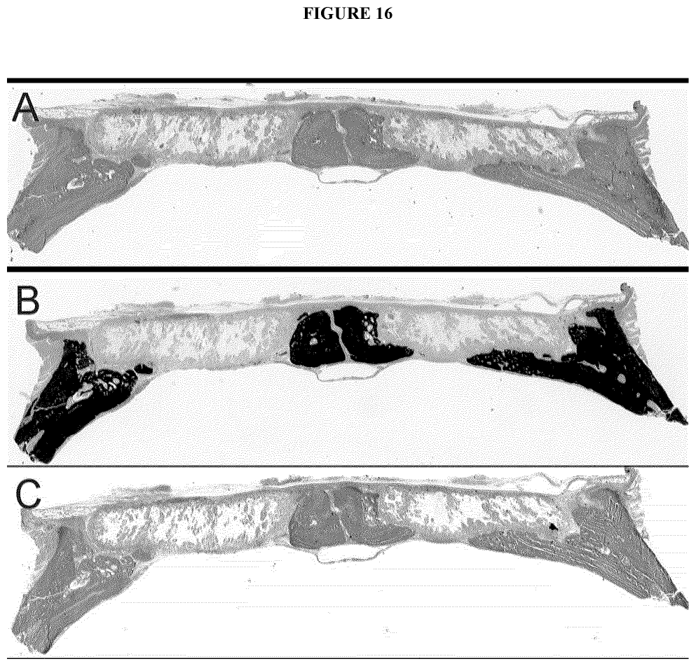

FIG. 16 shows histological section at 4 weeks after implantation (4WCH2). Hematoxylin and Eosin is shown in (A), Von Kossa/Van Gieson is shown in (B) and Masson Goldner Trichrome is shown in (C). Scale=2 mm for (A), (B) and (C).

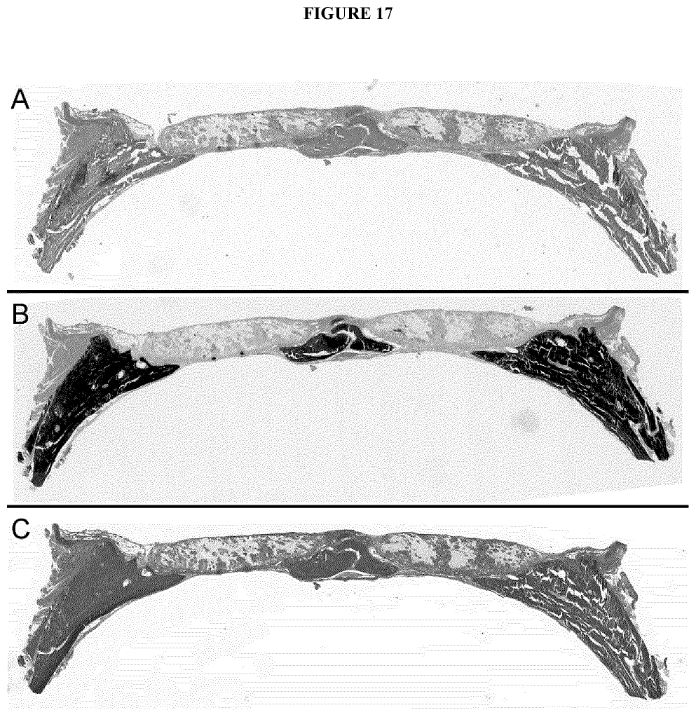

FIG. 17 shows histological section at 8 weeks after implantation (8WCH1). Hematoxylin and Eosin is shown in (A), Von Kossa/Van Gieson is shown in (B) and Masson Goldner Trichrome is shown in (C). Scale=2 mm for (A), (B) and (C).

FIG. 18 shows implantation in rat critical size calvarial defect model. Perforated 5 mm diameter by 1 mm thickness biomaterial is shown in (A). Implantation of the biomaterial into bilateral defects is shown in (B). On the left, the biomaterial is implanted, empty defect on the right-hand side.

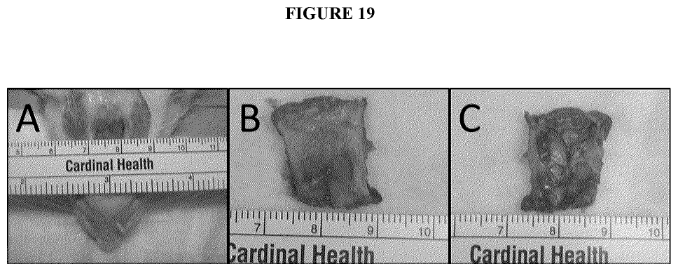

FIG. 19 shows tissue removal after 8-week implantation. Before complete resection of the calvarium is shown in (A). Top view of the resected calvarium is shown in (B). Bottom view of the resected calvarium is shown in (C).

FIG. 20A-D shows interlocked composite of apples and carrots (SCC).

After characterizing the structural and mechanical properties, as well as the in vitro performance of apple-derived scaffolds in supporting the differentiation of pre-osteoblasts (see Example 1 for further detail), this study was performed to investigate how such scaffolds perform in vivo [33]. The common rat calvarial defect model was employed to study how well the scaffolds would integrate into bone. Craniotomies were performed on Sprague-Dawley rats. Bilateral, 5 mm defects were created in both parietal bones and bare, acellular, apple-derived cellulose scaffolds were implanted in the defects (FIG. 18A, B). The implants were left for eight weeks and after euthanasia the top section of the skull was retrieved and processed for either histology or mechanical assessment.

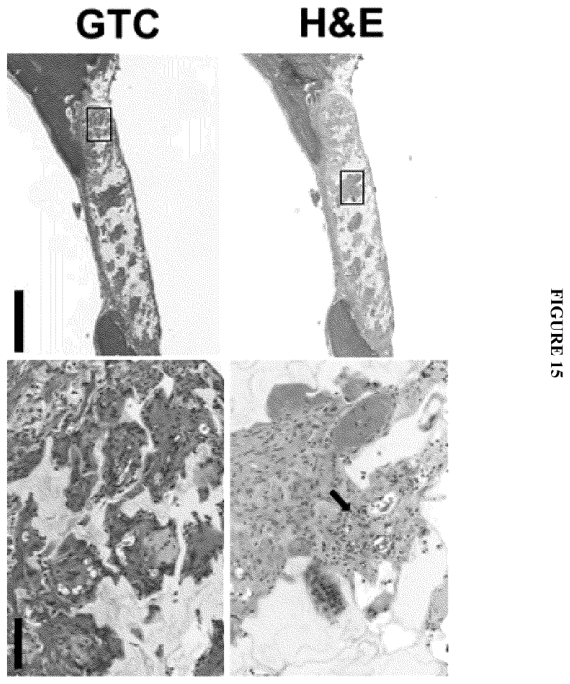

After eight weeks, upon visual inspection, the scaffolds appeared to have been well infiltrated with surrounding tissues from the skull. Therefore, to quantitatively measure how well the scaffolds had integrated with the bone tissues, mechanical push out tests were performed. Measurements of the grafted implants were immediately assessed following euthanasia of the animal using a uniaxial compression device (FIG. 14B). Briefly, a plunger is pushed against the scaffold and a load cell allows for the measurement of the force required to dislodge the implant. Results reveal that the average force required to dislocate the implant from the surrounding bone in this study is 114±28 N. Finally, histological sectioning and staining were used to evaluate cell infiltration and extracellular matrix deposition within the implanted grafts after eight weeks in the animals (FIG. 15). H&E staining showed infiltration of cells within the pores of the implants. There is also morphological evidence of vascularization within the scaffold, consistent with our previous animal studies [15], [16]. GTC staining showed a significant presence of type 1 collagen within the implants. Taken together the results support use of these scaffolds for use in bone tissue engineering applications.

Methods:

Scaffold production for the scaffolds shown in FIG. 20 was performed generally as already described in the examples hereinabove, and shapes were cut out with a CNC milling machine. Briefly, McIntosh Red apples (Canada Fancy) were cut to create two flat parallel faces. The apple was cut into peg (5 mm×5 mm×2 mm with a 2 mm peg extending from the centre) and hole (5 mm×5 mm×2 mm with a 2 mm diameter hole in the centre) Lego pieces with a Carbide 3D Shapeoko 3 CNC machine and the Chilipeppr jpadie software. The scaffolds were cut at a speed of 1 mm/s with a 0.8 mm diameter drill bit and an angle of 180°. The subunits were designed using Inkscape and were converted into the gcode using Jscut. The samples were removed from the bulk apple tissue by inverting and slicing on a Mandolin slicer set the to appropriate thickness (4 mm for the pegs and 2 mm for the holes). The samples were transferred to a 0.1% SDS solution and decellularized for 72 h while being shaken at 180 RPM. After decellularization, the samples were washed three times with dH₂O. Next, the subunits were incubated in 100 mM CaCl₂ for 24 h at room temperature to remove any surfactant residue. The samples were washed three times with dH₂O to remove the salt residues, and then they were incubated with 70% ethanol for sterilization. After the removal of the ethanol, three washes with dH₂O were performed to yield sterile scaffolds, free of contaminants. For the stress shielding experiments, carrots were cut into the hole subunit shapes as described above.

Scaffolds were subcutaneously implanted in rat (N=1 rat) in three different regions on the back. The implants were retrieved after 4 weeks and processed for histological slicing and staining. Histological staining is shown in FIG. 19.

### EXAMPLE 5

### Composite Scaffolds of Hyaluronic Acid and Alginate Cast Around Decellularized Apple for Osteoblast Differentiation

The present study shows that decellularized apple scaffolds combined with hyaluronic acid gels or alginate gels are suitable biomaterials for osteoblast culture. The differentiation of MC3T3 E1 subclone 4 pre-osteoblasts was accomplished. Calcium deposition and alkaline phosphatase activity were detected.

Biomaterial Formulations

For this study, composite scaffolds for cell culture were made using decellularized AA (apple) material as described herein. The decellularization process began with slicing and peeling McIntosh apples to 1 mm thick slices; these slices were then incubated in 0.1% sodium dodecyl sulfate (SDS) for 3 days, with incubation solutions being changed daily to fresh SDS. After the third day of SDS incubation, AA slices were washed with distilled water 3 times and incubated in 0.1 M calcium chloride (CaCl₂) for 1 day. On the following day, the slices were washed with water 3 times and sterilized through incubation in 70% ethanol (EtOH) for 30 minutes. After sterilization, AA slices were given another 3 water washes and stored in distilled water. Scaffolds for cell culture were then made using a sterile, 4 mm biopsy punch to stamp out circular pucks from the decellularized AA slices. The samples were stored in the appropriate cell culture media (i.e., α-MEM) in the refrigerator at −4° C. until used for cell seeding.

For cell culture on these AA composite scaffolds, two different hydrogels were prepared for MC3T3 cells to be resuspended within prior to cell seeding: hyaluronic acid (HA) and alginate. For the HA hydrogel pucks, HA solution was prepared in advance using Advanced BioMatrix HyStem Kit. For the alginate hydrogel pucks, a 0.5% alginate solution (saline-based and autoclaved) was prepared in advance and heated to 37° C. prior to cell culture; after cells were resuspended in the alginate solution and seeded onto pucks, the gels were then chemically crosslinked with the addition of 0.1M CaCl₂.

Cell Culture

MC 3T3 E1 Subclone 4 pre-osteoblast cells were cultured in MEM-alpha supplemented with 10% fetal bovine serum and 1% penicillin/streptomycin (100 U/mL and 100 μg/mL respectively). In order to invoke differentiation of the pre-osteoblasts, 4 mM inorganic phosphate (Sigma) and 50 μg/mL acetic acid (Sigma) were added. For subculturing, cells cultured on cell culture plates were trypsinized and resuspended in the appropriate medium. The cells were counted and centrifuged in order to separate the cells from the trypsin and the media. The supernatant was aspirated, and the cells were resuspended in the appropriate medium. 2.5×10⁴ cells were seeded onto the scaffold on day 1 and day 7. The cells were allowed to proliferate and invade the scaffold for 2 weeks prior to changing to differentiation medium for an additional 2 weeks. The culture media was replaced every second day.

Fixation, Staining, and Imaging

Alkaline Phosphatase Staining:

Prior to fixation, the scaffolds were washed with PBS. They were then fixed for 90 s with 3.5% paraformaldehyde and then washed with wash buffer (i.e. 0.05% Tween in PBS). The BCIP-NBT SigmaFast™ tablets were used; each tablet was dissolved in 10 mL of dH₂O. The BCIP concentration was 0.15 mg/mL, the NBT concentration was 0.3 mg/mL, the Tris buffer concentration was 100 mM, the MgCl₂ concentration was 5 mM, and the pH was between 9.25 and 9.75. During the staining, the samples were kept into the dark and were monitored. Once the staining was complete (5-10 min), the samples were washed and photographed. Staining and imaging were completed within one hour of making the staining solution.

Alizarin Red S Staining:

Prior to staining, the samples were fixed as outlined above, except the duration of the fixation process was 1 h. The biomaterials were then washed with PBS. Calcium staining was performed with a pre-made MilliporeSigma Alizarin Red S stain at pH 4.1±0.1. The samples were submerged in the stain and incubated for 45 min. Following the calcium staining, the samples were thoroughly washed with dH₂O until the colour ceased to run out of the samples. The samples imaged shortly afterwards.

Results

Alizarin Red S:

The samples were composite materials of decellularized apple scaffolds and either hyaluronic acid (HyStem Kit) or alginate cross-linked with CaCl₂.

Briefly, the cells were fixed and washed with PBS. Alizarin Red S was added to completely cover the samples (pH 4.2) for 45 min. The stain was then removed and the samples were washed gently but thoroughly with distilled water until the colour ceased to run.

The samples that were stained were the pre-differentiated alginate and hyaluronic acid materials as well as the differentiated materials. A strong red colour was indicative of calcium deposition. Both the differentiated samples exhibited this colour after staining. The control hyaluronic acid sample did not. The control alginate sample displayed an intermediate red colour, as calcium is the crosslinking agent in the hydrogel. Nevertheless, the alginate control was not as dark as the differentiated sample, which indicated that calcium deposition from mineralization due to differentiation occurred.

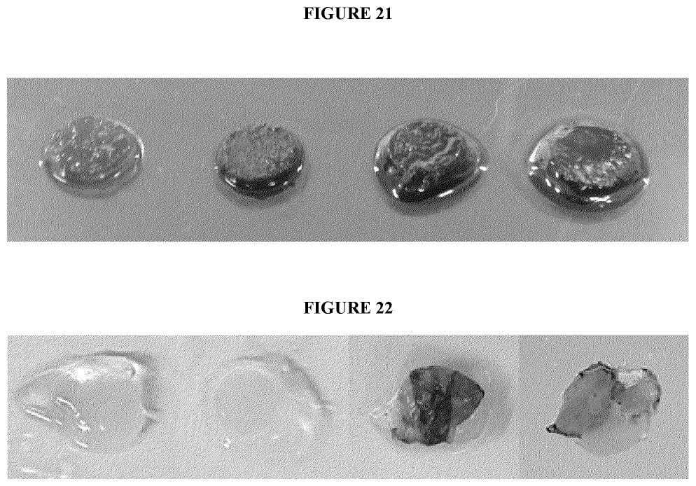

FIG. 21 shows Alizarin Red S staining for calcium deposition in MC3T3 E1 cell-laden composites. Left to right: hyaluronic acid and decellularized apple (pre-differentiation), alginate and decellularized apple (pre-differentiation), hyaluronic acid and decellularized apple (post-differentiation), alginate and decellularized apple (post-differentiation).

Alkaline Phosphatase:

A short fixation time was used for the alkaline phosphatase assays to prevent loss of enzyme activity. The samples were fixed for 90 s with 3.5% paraformaldehyde and were then washed with 0.05% Tween in PBS. The BCIP-NBT SigmaFast™ tablets were dissolved in dH₂O to create the ready-to-use staining solution. The purple colour is indicative of alkaline phosphatase activity, which is a marker for osteoblast differentiation in this context.

The samples that were stained were the pre-differentiated alginate and hyaluronic acid materials as well as the differentiated materials. Both the differentiated samples exhibited the purple colour after staining. The control hyaluronic acid and alginate samples did not.

FIG. 22 shows Alkaline phosphatase staining with BCIP NBT SigmaFast™ tablets in MC3T3 E1 cell-laden composites. Left to right: hyaluronic acid and decellularized apple (pre-differentiation), alginate and decellularized apple (pre-differentiation), hyaluronic acid and decellularized apple (post-differentiation), alginate and decellularized apple (post-differentiation).

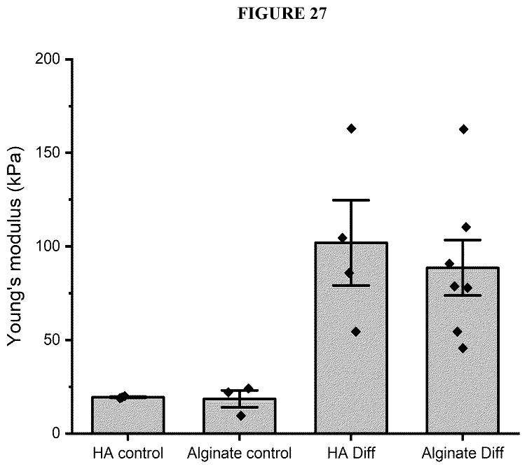

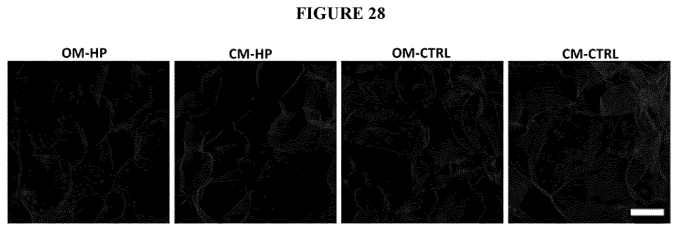

Based on the results collected, it is predicted that the stiffness of the differentiated samples is greater than the undifferentiated samples. Indeed, FIG. 27 provides results showing that decellularized apple scaffolds combined with hyaluronic acid gels or alginate gels are suitable biomaterials for osteoblast culture. The differentiation of MC3T3 E1 subclone 4 pre-osteoblasts was accomplished. Calcium deposition and alkaline phosphatase activity were detected, and an increased stiffness was attained. Mechanical testing: The Young's modulus was calculated from the linear region of the stress-strain curves. There was no statistically significant difference between the gel types, nor were there any statically significant interactions in the two-way ANOVA (p=0.05). However, there was a significant difference between the controls and the differentiated samples (p=8.9×10⁻⁴). Due to the uneven initial contact area and the composite nature of the materials, there was a toe region at the beginning of the stress-strain curves. The analysis was performed after this toe region. FIG. 27 shows Young's modulus of decellularized AA (apple) with hyaluronic acid (HA) or alginate hydrogels without cells (control) and with cells after differentiation (Diff). Descriptive statistics of the Young's moduli for the control and differentiated samples are as follows:

| Sample | Gel | N | Mean | SD | SEM | Variance |
|---|---|---|---|---|---|---|
| Control | HA | 3 | 19435.56 | 539.1605 | 311.2844 | 290694 |
| Control | Alginate | 3 | 18551.98 | 7879.218 | 4549.069 | 6.21E+07 |
| Differentiated | HA | 4 | 101935.9 | 45630.33 | 22815.16 | 2.08E+09 |
| Differentiated | Alginate | 7 | 88585.07 | 39145.76 | 14795.71 | 1.53E+09 |

The above results and those in FIG. 27 show that the composite materials made from decellularized apple scaffolds and a hydrogel cast around the material, either hyaluronic acid or alginate in this example, can act as viable scaffolds for osteoblast differentiation and bone tissue engineering.

The results of this study support that the composite materials made from decellularized scaffolds (such as those derived from apple as described herein) and a hydrogel cast around the material, either hyaluronic acid or alginate in this example, can act as viable scaffolds for osteoblast differentiation and bone tissue engineering.

### EXAMPLE 6

### Effects of Hydrostatic Compression on Native Cellulose Scaffolds for Bone Tissue Engineering

Upon injury or break, bones have the ability of self renewal. However, large defects created by either injury or disease may require graft placement to avoid non-union or malunion of the bone tissue [39]. Such grafts can be derived from the patient's own body (autologous grafts), usually the iliac crest, which is considered the “gold standard” in regenerative orthopedics [40]-[43]. However, limited size grafts, donor site morbidity and infections, cost and post-operative pain at both donor and receiver site may lead to alternative sources for the graft [41], [42]: from a cadaver donor (allograft), from animal sources (xenograft), or artificially derived (alloplastic). Such alternatives all have their own benefits and drawbacks, the later however may provide a potential alternative with lower risk of transmitted diseases and infections, as well as overcoming the size limitation barrier [41], [42]. Alloplastic graft is also considered a more ethical alternative than allografts and xenografts [44]. Physical properties are key parameters for alloplastic grafts development, such as pore size, pore interconnectivity and elastic modulus [43], [45], [46]. Fine tuning of these parameters may lead to better mechanical support, stability of the implant, and/or may lead to improved osteoconductivity and osteoinductivity. Thus, designing such materials for bone tissue engineering (BTE) applications may benefit from fine tuning according to the surrounding environment.

Long bones are highly dynamic structural tissues, with functions ranging from body support to physical locomotion. A whole spectrum of forces is acting on different areas of the skeletal system. For instance, the pressure found in femur head in human adult can reach 5 MPa during normal locomotion, and can reach up to 18 MPa for other activities [47]. On a microscopic level, these forces are transmitted to the osteocytes through Wnt/β-catenin mechano-sensing pathways in the lacuna-canaliculi network [48]. Such force-regulated mechanisms lead to formation and removal of bone tissue, trough bone remodeling processes [48]. It's been shown that the pressure inside the lacuna-canaliculi network is around 280 kPa [49]. Bioreactors are under development to apply stresses to cultured osteoblast cells (and their underlying substrate) to better replicate native bone environment. Such bioreactors can apply contact uniaxial compression/tension , contact biaxial compression/tension, flow inducing shear-stress, mechanical shear stress electrical or a combination of these stimuli [50], [51]. Also, bioreactors applying static or cyclic hydrostatic pressure by compressing the gas phase above incompressible media, or by direct compression of the medium may be used on seeded cells [52]-[58].

In addition to mechanical stimulus, three-dimensional culturing of the cells is desirable for better representing the in vivo conditions. Three-dimensional structures may support the growth and proliferation of cells and may mimic the extracellular matrix found in specific tissues. With a specific tissue-oriented scaffold structure (or biomaterial) and appropriate applied mechanical stimuli it is contemplated that better osteointegration and overall performance in vivo may be realized. Cellulose-base scaffolds derived form plants can be used as tissue engineering scaffolds [59]-[61]. These biomaterials can be sourced from plants that closely matches the microstructure of the tissue to be replicated [61]. Successful experiments in vitro and in vivo showed that these biomaterials can host various cell types , are biocompatible and supports active angiogenesis [59]-[61]. Scaffolds can be mineralized by differentiated osteoblasts [62]. Moreover, some scaffolds can be artificially mineralized by soaking them in simulated body fluid [63].

In this study, the effects of increased atmospheric pressure (by applied cyclic hydrostatic pressure) on the differentiation capacity of pre-osteoblastic cells cultured on apple-derived 3D scaffold biomaterials is examined. Cells were exposed to a cyclic pressure cycle (max 280 kPa, 1 Hz) for one hour per day, for a total of two weeks. The results generally reveal that in osteogenic media the cells pressure cycling leads to an increase in the number of cells, Alkaline phosphatase (differentiation marker) activity and mineralization over time.

Materials and Methods

Scaffold Fabrication

Samples were prepared following protocols as described herein. Briefly, MacIntosh apples (Canada Fancy) were cut with a mandolin slicer to 1 mm-thick slices. A biopsy punch (Fisher) was used to create 5 mm-diameter disks in the hypanthium tissue of the apple slices. The disks were decellularized in a 0.1% sodium dodecyl sulfate solution (SDS, Fisher Scientific, Fair Lawn, N.J.) for two days. Then, the decellularized disks were gently washed in deionized water, before incubation in 100 mM CaCl₂ for two days. The samples were sterilized with 70% ethanol for 30 min, gently washed in deionized water, and placed in a 96-well culture plate prior to cell seeding.

MC3T3-E1 Subclone 4 cells (ATCC® CRL-2593TM, Manassas, Va.) [64] were cultured and maintained in a humidified atmosphere of 95% air and 5% CO₂, at 37° C. The cells were cultured in Minimum Essential Medium (α-MEM, ThermoFisher, Waltham, Mass.), supplemented with 10% Fetal Bovine Serum (FBS, Hyclone Laboratories Inc., Logan, Utah) and 1% Penicillin/Streptomycin (Hyclone Laboratories Inc). Cells were tryspinized and suspended in culture media. Scaffolds were placed individually in 96-well plates. Prior to cell seeding, scaffolds were immersed in culture media and incubated in a humidified atmosphere of 95% air and 5% CO₂, at 37° C., for 30 min. The culture media was completely aspirated from the wells. Cells were tryspinized and suspended and a 30 μL drop of cell culture suspension, containing 5·10⁴ cells, was pipetted on each scaffold. The cells were left to adhere on the scaffolds for 2 hours before adding 200 μL of culture media to the culture wells. Culture media was changed every 3-4 days for 1 week. Cells seeded scaffolds were then either incubated in osteogenic media (OM) by adding 50 μg/mL of ascorbic acid and 10 mM β-glycerophosphate to the culture media or incubated in culture media (CM) for 2 weeks, with or without the application of hydrostatic pressure (HP).

Cyclic Hydrostatic Pressure Stimulation

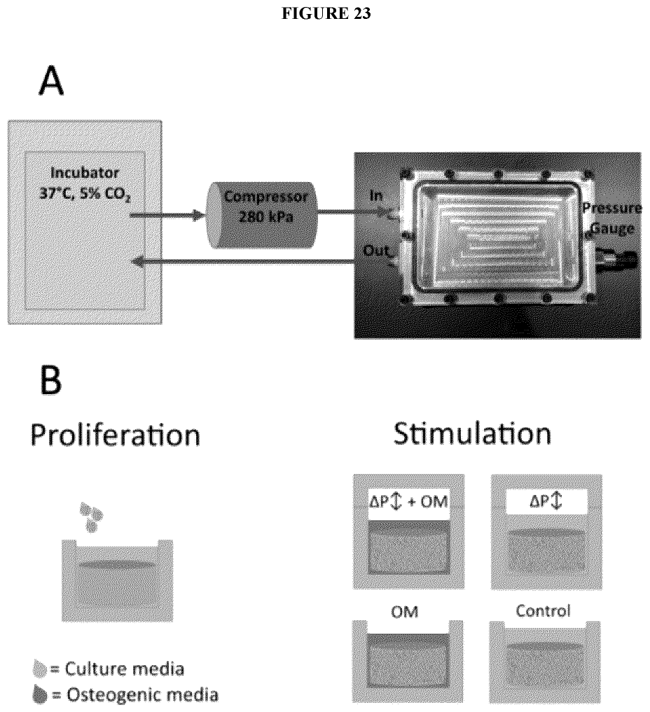

Cyclic hydrostatic pressure was applied by modulating the pressure in the gas phase above the culture wells in a custom-build pressure chamber (FIG. 23, A). Briefly, the humidified, 95% air and 5% CO₂ incubator atmosphere was compressed using a compressor (Mastercraft) and injected in the pressure chamber using solenoid valves. A microcontroller (Particle Photon) was used to control the frequency and the duration of the applied pressure remotely via a custom-made cellphone application. Cyclic hydrostatic pressure stimulation was applied during 1 hour per day, for up to 2 weeks (FIG. 23, B) at a frequency 1 Hz, oscillating between 0 and 280 kPa with respect to ambient pressure. Pressure was monitored using a pressure transducer. The samples were removed from the pressure chamber after each cycle and kept at ambient pressure between the stimulation phases.

Cell-seeded scaffolds were either stimulated with cyclic hydrostatic pressure with and without the presence of osteogenic media, leading to four experimental conditions (FIG. 23, B): Cyclic hydrostatic pressure in regular culture media (CHP), cyclic hydrostatic pressure in osteogenic culture media (CHP-OM), non-stimulated in osteogenic media (OM) and non-stimulated in regular culture media (control). The OM and control conditions were kept outside of the pressure chamber, in a humidified, 5% CO₂ incubator at 37° C.

Scaffold Imaging

After 1 week or 2 weeks, scaffolds were thoroughly washed with PBS and fixed with 10% neutral buffered formalin for 10 min. Scaffolds were washed with PBS and incubated in a 0.01% Congo Red staining solution (Sigma) for 20 min at room temperature. Scaffolds were washed 3 times with PBS. Cell nuclei were stained with 1:1000 Hoechst (ThermoFisher) for 30 min in the dark. Samples were washed 3 times with PBS and stored in wash buffer solution (5% FBS in PBS) prior to imaging. The cell-seeded surface of the scaffolds was imaged with a high-speed resonant laser scanning confocal microscope (Nikon Ti-E A1-R) equipped with a 10× objective. Maximum intensity projections of the image slices were used for cell counting with ImageJ software [65]. Cells were counted on a 1.3 by 1.3 mm² area (N=3 per experimental conditions with 3 randomly selected area per scaffold).

Alkaline Phosphatase Activity Assay

Alkaline phosphatase (ALP) activity in media was measured using an ALP assay kit (BioAssay Systems, Hayward, Calif.). Briefly, a working solution was prepared to a 5 mM magnesium acetate and 10 mM pNPP concentration in assay buffer, following manufacturer's protocol. 150 μL of working solution was pipetted in 96-well plate. 200 μL of calibrator solution and 200 μL of dH₂O were pipetted in separated well, in the same 96-well plate. At 1 week and 2 weeks, 20 μL of incubation media (either CM or OM) was pipetted into the working solution's well. All wells (samples, calibrator and dH₂O) were read at 405 nm for 10 minutes, every 30 seconds. ALP activity was calculated by taking the slope of the 405 nm readings vs time, calibrated with the calibrator solution and dH₂O. Wells were read in triplicates (N=3 per experimental conditions).

Alizarin Red S Staining and Mineral Deposit Quantification

Samples were fixed with 10% neutral buffered formalin for 10 min, after 1 week or 2 weeks. Calcium quantification was performed using established protocol (C. A. Gregory, W. G. Gunn, A. Peister, and D. J. Prockop, “An Alizarin red-based assay of mineralization by adherent cells in culture: Comparison with cetylpyridinium chloride extraction,” Anal. Biochem., vol. 329, no. 1, pp. 77-84, June 2004, which is herein incorporated by reference in its entirety, [66]). Briefly, samples were transferred to a 24-well plate and carefully washed with deionized water and incubated in 1 mL of 40 mM (pH=4.1) alizarin red s (ARS) solution for 20 minutes at room temperature, with light agitation. The samples were then washed 3× with deionized water and placed in 15 mL falcon tubes filled with 10 mL dH₂O. The tubes were placed on a rotary shaker at 120 rpm for 60 min and dH₂O was replaced every 15 min. Thereafter, samples were incubated in 800 μL of 10% acetic acid on an orbital shaker at 60 rpm for 30 min. The eluted ARS/acetic acid solution was pipettes out of the well and transferred to 1.5 mL centrifuge tubes. Tubes were centrifuged at 17 10⁴ g for 15 min. 500 μL of supernatants were transferred to new centrifuge tube and 200 μL of 10% ammonium hydroxide was pipetted into the tubes. Finally, 150 μL of the solution was pipetted into a 96-well plate and the absorption at 405 was read using a plate reader.

Wells were read in triplicates (N=3 per experimental conditions).

Young's Modulus Measurements

Young's modulus measurements of the scaffolds were performed using a custom-built uniaxial compression apparatus, following method previously described [61]. Briefly, after 1 week or 2 weeks, the scaffolds were mechanically compressed at a rate of 3 mm min' to a maximum strain of 10%. The force vs displacement curves were recorded a 500 g load cell (Honeywell, Charlotte, N.C.) and an optical ruler (Honeywell). The Young's modulus of the scaffolds under the different experimental conditions were obtained by fitting the linear portion of the resulting stress-strain curve.

Statistical Analysis

Values reported in this Example are the average value±standard error of the mean (SEM). Statistical significance was determined using one-way ANOVA and post hoc Tukey test. A value of p<0.05 was considered to be statistically significant.

Results

FIG. 23 shows (A) Cyclic hydrostatic pressure device schematics. Hydrostatic pressure was applied by modulating the pressure in the gas phase above the culture wells in a custom-build pressure chamber. Air from incubator atmosphere was compressed using a compressor and injected in the pressure chamber using solenoid valves. (B) shows experimental conditions. After 1 week of proliferation, cyclic hydrostatic pressure stimulation was applied during 1 hour per day, for up to 2 weeks at a frequency 1 Hz, oscillating between 0 and 280 kPa with respect to ambient pressure. The samples were removed from the pressure chamber after each cycle and kept at ambient pressure between the stimulation phases.

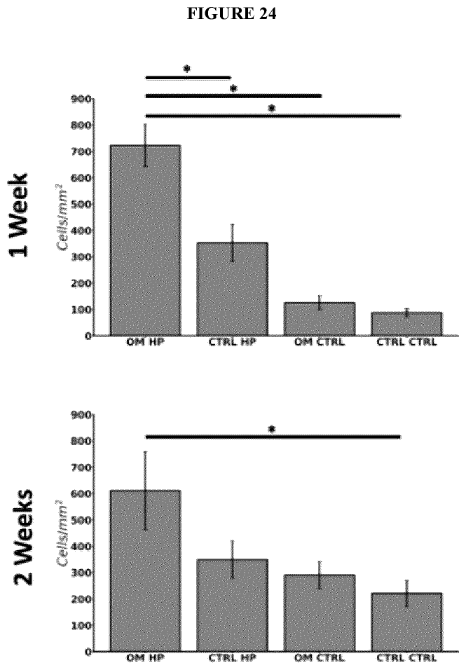

FIG. 24 shows cellular density after 1 week or 2 weeks of stimulation. Statistical significance (* indicates p<0.05) was determined using a one-way ANOVA and Tukey post-hoc tests. Data are presented as means±S.E.M. of three replicate samples per condition, with three areas per sample. The results reveal that after 2 weeks in culture, there are significantly more cells present on scaffolds which experienced cyclic pressure loading compared to controls.

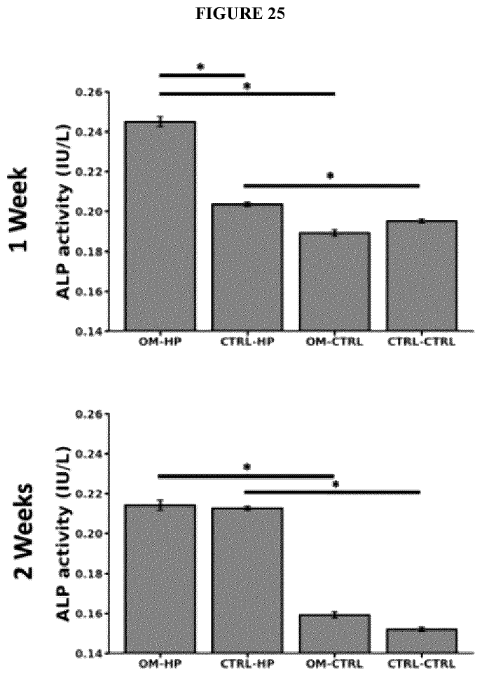

FIG. 25 shows alkaline phosphatase (ALP) activity after 1 week or 2 weeks of stimulation. Statistical significance (* indicates p<0.05) was determined using a one-way ANOVA and Tukey post-hoc tests. Data are presented as means±S.E.M. of three replicate samples per condition. The results reveal that after 2 weeks in culture, there is significantly ALP activity (a marker of differentiation) in cells present on scaffolds which experienced cyclic pressure loading compared to controls.

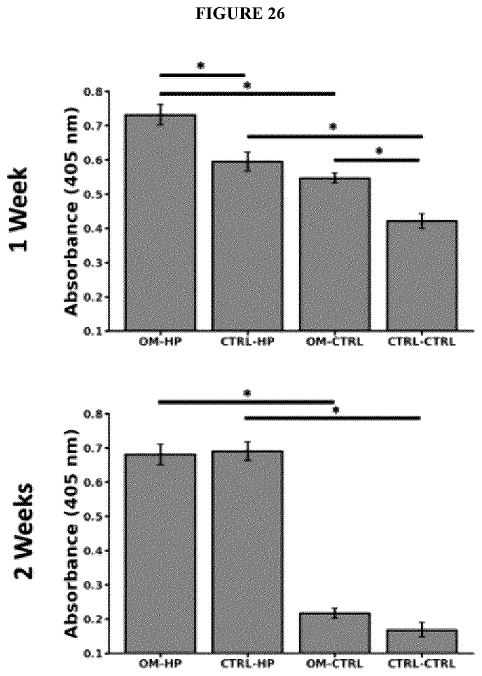

FIG. 26 shows mineral deposit quantification with Alizarin Red S (ARS) staining after 1 week or 2 weeks of stimulation. Statistical significance (* indicates p<0.05) was determined using a one-way ANOVA and Tukey post-hoc tests. Data are presented as means±S.E.M. of three replicate samples per condition. The results reveal that after 2 weeks in culture, there is significantly more mineralization of the scaffolds which experienced cyclic pressure loading compared to controls.

Scaffold Imaging and Cell Counting:

Cell counting was performed on maximum projection of confocal slices (FIG. 28, 24). Data showed (FIG. 28, 24) significant increase in cellular density in scaffolds incubated in OM compared to CM, subjected to hydrostatic pressure after 1 week (723±80 cells/mm² and 353±71 cells/mm², respectively; p=0.02) but showed a non-significant increase after 2 weeks of stimulation (611±149 cells/mm² and 350±71 cells/mm², respectively; p=0.23). Non-significant increase was also observed in cellular density in scaffolds incubated in OM compared to CM, in the static case 1 week (125±27 cells/mm² and 88±16 cells/mm², respectively; p=0.99) and 2 weeks of stimulation (291±52 cells/mm² and 221±50 cells/mm², respectively; p=0.99). The application of hydrostatic pressure significantly increases the density of cells after for scaffolds incubated in OM after 1 week of stimulation compared to the static case (723±80 cells/mm² and 125±27 cells/mm², respectively; p=10-5). An increase, non-significant, was also observed after 2 weeks of stimulation in similar conditions (611±149 cells/mm² and 291±52 cells/mm², respectively; p=0.07). Moreover, a non-significant increase in cellular density was observed by applied HP in scaffolds cultured in CM after 1 week (353±71 cells/mm² and 88±16 cells/mm², respectively; p=0.21) and 2 weeks of stimulation (350±71 cells/mm² and 221±50 cells/mm², respectively; p=0.92). In respective experimental condition, no significant change in the cell density were observed between the first and second week for scaffolds subjected to HP (723±80 cells/mm² and 611±149 cells/mm² for OM-HP scaffolds; p=0.96; 353±71 cells/mm² and 350±71 cells/mm² for CTRL-HP scaffolds; p=1). Finally, no significant change in the cell density were observed between the first and second week for scaffolds in the static case (125±27 cells/mm² and 291±52 cells/mm² for CM-HP scaffolds; p=1; 88±16 cells/mm² and 221±50 cells/mm² for CM-CTRL scaffolds; p=0.91).

Alkaline Phosphatase Activity Assay:

Alkaline phosphatase activity was assed by a pnpp kinetic reaction following manufacturer's protocol after 1 or 2 weeks (FIG. 25). A significant increase in ALP activity was observed in scaffolds with applied hydrostatic pressure compared to the static case, incubated in osteogenic media after 1 week (0.245±0.003 IU/L and 0.189±0.002 IU/L, respectively; p=4×10⁻⁸) and 2 weeks (0.214±0.002 IU/L and 0.159±0.002 IU/L, respectively; p=4×10⁻⁸) of stimulation. Moreover the application of hydrostatic pressure also significantly increased the ALP activity in culture media after 1 week (0.203±0.001 IU/L and 0.195±0.001 IU/L, respectively; p=0.03) and 2 weeks (0.213±0.001 IU/L and 0.152±0.001 IU/L, respectively; p=5×10⁻⁸). ALP activity significantly increased in samples incubated in osteogenic media compared to culture media with applied hydrostatic pressure after 1 week (0.245±0.003 IU/L and 0.203±0.001 IU/L, respectively; p<10-8) but was not significantly different after 2 weeks (0.159±0.002 IU/L and 0.152±0.001 IU/L, respectively; p=0.99). Finally, ALP dis not significantly change in the absence of hydrostatic pressure for samples incubated in osteogenic media compared to culture media either at 1 week (0.189±0.002 IU/L and 0.195±0.001IU/L, respectively; p=0.25) or 2 weeks (0.159±0.002 IU/L and 0.152±0.001 IU/L, respectively; p=0.08).

Alizarin Reds Staining and Mineral Deposit Quantification:
ARS assay for quantifying mineralization was performed after 1 or 2 weeks (FIG. 26). The application of hydrostatic pressure significantly increased the quantity of mineral deposition for both differentiation media (0.73±0.03 a.u. and 0.55±0.02 a.u., HP vs CTRL, respectively; p=2×10⁻⁷) or culture media (0.59±0.03 a.u. and 0.42±0.02 a.u., HP vs CTRL, respectively; p=1×10⁻⁶) at 1 week. The quantity of mineral deposition was also significantly increased after 2 weeks incubation in differentiation media (0.68±0.01 a.u. and 0.22±0.02 a.u., HP vs CTRL, respectively; p=2×10⁻⁸) and culture media (0.69±0.02 a.u. and 0.17±0.02 a.u., HP vs CTRL, respectively; p=2×10⁻⁸). The mineral deposition was also significantly increased by incubation in osteogenic media compared to culture media at 1 week for samples under hydrostatic pressure (0.73±0.03 a.u. and 0.59±0.03 a.u., OM vs CM; p=2×10⁻⁴) and for non-compressed experiments (0.55±0.02 a.u. and 0.42±0.02 a.u., OM vs CM; p=10⁻³). No significant change of mineral deposition was observed by incubation in osteogenic media compared to culture media at 2 weeks for samples under hydrostatic pressure (0.68±0.01 a.u. and 0.69±0.02 a.u., OM vs CM; p=0.99) and for non-compressed experiments (0.22±0.02 a.u. and 0.17±0.02 a.u., OM vs CM; p=0.75).

Young's Modulus Measurements:

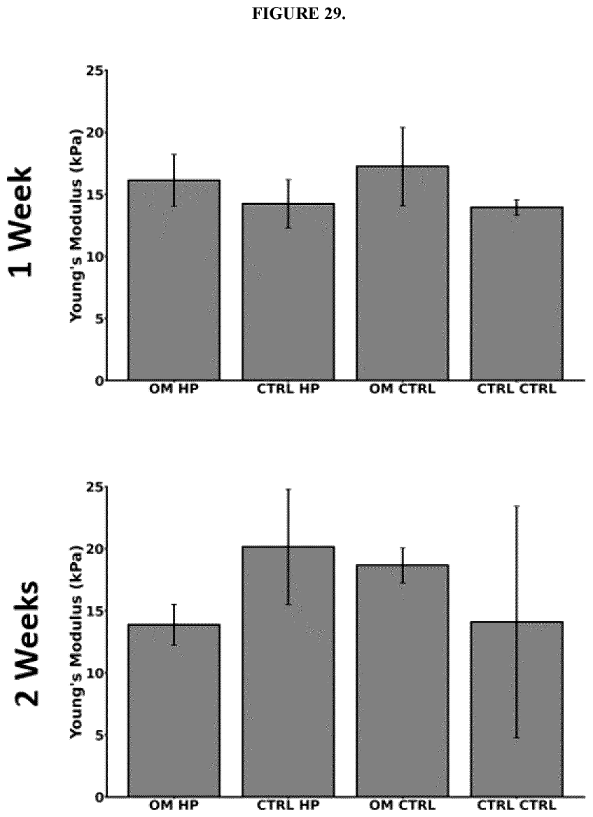

After 1 week or 2 weeks of stimulation, scaffolds were assessed for change in Young's modulus (FIG. 29). Data showed no significant changes between samples incubated in osteogenic media with applied hydrostatic pressure and without applied hydrostatic pressure after 1 week (0.016±0.002 MPa and 0.017±0.003 MPa, HP vs CTRL; p=0.99) or 2 weeks (0.014±0.001 MPa and 0.019±0.001 MPa, HP vs CTRL; p=0.85). Moreover, no significant changes in the Young's modulus was observed in samples incubated in culture media with applied hydrostatic pressure or without hydrostatic pressure, both after 1 week (0.014±0.002 MPa and 0.014±0.001 MPa, HP vs CTRL; p=1) or 2 weeks (0.020±0.002 MPa and 0.014±0.005 MPa, HP vs CTRL; p=0.64) of experiment. Furthermore, no significant change in the Young's modulus was observed between samples under applied hydrostatic pressure in osteogenic media and culture media at 1 week (0.016±0.002 MPa and 0.014±0.002 MPa, OM vs CM; p=0.99) or after 2 weeks (0.014±0.001 MPa and 0.020±0.002 MPa, OM vs CM; p=0.6). Similarly, no significant change in the Young's modulus was observed between samples at atmospheric pressure in osteogenic media and culture media at 1 week (0.017±0.003 MPa and 0.014±0.001 MPa, OM vs CM; p=0.98) or after 2 weeks (0.019±0.001 MPa and 0.014±0.005 MPa, OM vs CM; p=0.88).

Discussion

Close representation of physical environment is desirable for bone tissue recovery [41]-[43], [45], [46]. Similarly, close matching of surrounding bone tissue may be a key factor in the success of alloplastic grafts [45], [46]. Cellulose biomaterial derived from plan tissues that closely match the physical environment has shown promising results in vitro, in vivo for targeted tissue engineering [59]-[61]. In this Example, biomaterials are investigated by replicating the mechanical environment of human locomotion. External pressure was applied on the scaffolds in similar magnitude of the lacuna-canaliculi network with a frequency mimicking human locomotion (1 Hz) [49], [52]. Scaffolds were seeded with pre-osteoblasts cells (MC3T3-E1). After proliferation, scaffolds were either cultured in standard culture media (CM), or in osteogenic-inducing differentiation media (OM). These scaffolds were then either subjected to cyclic hydrostatic pressure (HP) or kept at atmospheric pressure (CTRL) for 1 or 2 weeks. The applied HP was set at 1 Hz for 1 hour per day, following a rest period at atmospheric pressure. Other groups using either similar cell line [57], [67], human or animal bone marrow skeletal stem cells (BMSCs) [54], [55], [58] or ex-vivo chick femur [52] reports the effects of cyclic HP on either 2D surfaces, random or aligning PCL meshes or ex vivo bones. This Example measured the effect of HP on Native Cellulose Scaffolds seeded with MC3T3-E1 cells. Cell counting by laser-scanning confocal microscopy revealed that the density of cells was significantly increased after 1 week and (non-significantly; p=0.07) 2 weeks of applied HP in osteogenic medium. An increase was also noted after 1 week and 2 weeks of applied HP in culture medium but was also non-significant. These results showed that the application of HP enhance MC3T3-E1 proliferation when cultured in OM. This result is corroborated by other studies [54], [55]. Using similar mechanical stimulation (270 kPa; 1 Hz stimulation for 1 h per day, for 5 days out of 2 weeks), Reinwald et al., 2018 showed that human BMSCs metabolic activity was upregulates in comparison to the non-stimulated samples [54]. Zhao et al., 2015 showed that the application of hydrostatic pressure on rat BMSCs accelerates cell proliferation through upregulated cell cycle initiation [55]. Similarly, Stavenschi et al., 2018 reported that physical stimulation of MC3T3-E1 cells induced expression of paracrine factors that leads enhancement of cell proliferation [58]. Physical stimulus thus affects the proliferation of cells in three-dimensional scaffolds. The nature of incubation media influence on cellular density was illustrated by a significant increase in OM samples compared to CM samples after 1 week of HP, but a non-significant increase after 2 weeks in HP. Non-significant increase between OM and CM was observed for non-stimulated samples after 1 and weeks. Quarles et al., 1992 also reported a time-dependent, significant increase in MC3T3 cell number cultured in media containing ascorbic acid and β-glycerophosphate [64]. More over, a time-dependent decrease in the replication rate was also reported [64]. Hong et al., 2010 reported a significant diminution of MC3T3-E1 cell incubated in similar osteogenic media compared to culture media, and no significant different at 2 weeks of culture [68]. Findings in the present Example further suggest that the application of HP influences the replication rate at early stages of stimulation for samples cultured in OM. Alkaline phosphatase is an enzyme expressed in early staged of osteoblastic differentiation [69]. The present results indicate that the application of cyclic hydrostatic pressure significantly increase the ALP activity of cell-seeded scaffolds, compared to the static case. A significant increase in ALP activity was also noted by the incubation of the scaffolds in osteogenic-inducing differentiation media, similarly to reports on 2D culture systems [64], [68]. The application of HP significantly increased the mineral content in the scaffolds after 1 week and 2 weeks of stimulation, in both type of incubation media. Stavenschi 2018 et al., showed that a cyclic 300 kPa pressure at 2 Hz frequency on human BMSCs promoted significant mineral deposition [58]. Henstock et al., 2013 also noted increase in mineral deposition in ex vivo bone samples, with similar hydrostatic pressure force application [52]. Furthermore, the incubation in osteogenic media increased the mineral content in the scaffolds, which is consistent with other studies in other systems [64], [68]. Along with ALP expression, mineral content expression further confirms the ongoing differentiation of MC3T3-E1 onto osteoblast, either by applied HP, chemically (induction in OM) or a combination of both. Finally, dynamical mechanical analysis revealed no significant change in the young's modulus between all experimental conditions, and between the first week and second week of experimentation. Young's modulus of similar scaffolds seeded with MC3T3-E1 displayed a much higher values for scaffolds incubated in osteogenic media than the ones here (OM-HP and OM-CTRL). The duration of the incubation in osteogenic media and initial seeding density were different, which may explain the difference between the values.

In this Example examine the effects of increased atmospheric pressure on the differentiation capacity of pre-osteoblastic cells cultured on apple-derived 3D scaffold biomaterials was examined. Cells were exposed to a cyclic pressure cycle (max 280 kPa, 1 Hz) for one hour per day, for a total of two weeks. The results reveal that application of hydrostatic pressure, in combination with osteogenic inducing media, leads to an increase in the number of cells, Alkaline phosphatase (differentiation marker) activity, and mineralization over time.

One or more illustrative embodiments have been described by way of example. It will be understood to persons skilled in the art that a number of variations and modifications can be made without departing from the scope of the invention as defined in the claims.

### REFERENCES

[1] J. P. Schmitz and J. O. Hollinger, “The critical size defect as an experimental model for craniomandibulofacial nonunions.,” Clin. Orthop. Relat. Res., no. 205, pp. 299-308, April 1986.

[2] X. Yu, X. Tang, S. V Gohil, and C. T. Laurencin, “Biomaterials for Bone Regenerative Engineering.,” Adv. Healthc. Mater., vol. 4, no. 9, pp. 1268-85, June 2015.

[3] S. N. Parikh, “Bone graft substitutes: past, present, future.,” J. Postgrad. Med., vol. 48, no. 2, pp. 142-8.

[4] J. S. Silber et al., “Donor site morbidity after anterior iliac crest bone harvest for single-level anterior cervical discectomy and fusion.,” Spine (Phila. Pa. 1976)., vol. 28, no. 2, pp. 134-9, January 2003.

[5] A. R. Amini, C. T. Laurencin, and S. P. Nukavarapu, “Bone tissue engineering: recent advances and challenges.,” Crit. Rev. Biomed. Eng., vol. 40, no. 5, pp. 363-408, 2012.

[6] D. L. Butler, S. A. Goldstein, and F. Guilak, “Functional tissue engineering: the role of biomechanics.,” J. Biomech. Eng., vol. 122, no. 6, pp. 570-5, December 2000.

[7] V. Karageorgiou and D. Kaplan, “Porosity of 3D biomaterial scaffolds and osteogenesis,” Biomaterials, vol. 26, no. 27, pp. 5474-5491, September 2005.

[8] S. Bose, M. Roy, and A. Bandyopadhyay, “Recent advances in bone tissue engineering scaffolds.,” Trends Biotechnol., vol. 30, no. 10, pp. 546-54, October 2012.

[9] H. Yoshikawa and A. Myoui, “Bone tissue engineering with porous hydroxyapatite ceramics,” J. Artif. Organs, vol. 8, no. 3, pp. 131-136, October 2005.

[10] Q. Fu, E. Saiz, M. N. Rahaman, and A. P. Tomsia, “Bioactive glass scaffolds for bone tissue engineering: state of the art and future perspectives.,” Mater. Sci. Eng. C. Mater. Biol. Appl., vol. 31, no. 7, pp. 1245-1256, October 2011.

[11] I. D. Xynos, A. J. Edgar, L. D. K. Buttery, L. L. Hench, and J. M. Polak, “Ionic Products of Bioactive Glass Dissolution Increase Proliferation of Human Osteoblasts and Induce Insulin-like Growth Factor II mRNA Expression and Protein Synthesis,” Biochem. Biophys. Res. Commun., vol. 276, no. 2, pp. 461-465, September 2000.

[12] R. Kroeze, M. Helder, L. Govaert, and T. Smit, “Biodegradable Polymers in Bone Tissue Engineering,” Materials (Basel)., vol. 2, no. 3, pp. 833-856, July 2009.

[13] J. Venkatesan, P. A. Vinodhini, and P. N. Sudha, “Chitin and Chitosan Composites for Bone Tissue Regeneration,” Adv. Food Nutr. Res., vol. 73, pp. 59-81, January 2014.

[14] D. J. Modulevsky, C. Lefebvre, K. Haase, Z. Al-Rekabi, and A. E. Pelling, “Apple Derived Cellulose Scaffolds for 3D Mammalian Cell Culture,” PLoS One, vol. 9, no. 5, p. e97835, May 2014.

[15] D. J. Modulevsky, C. M. Cuerrier, and A. E. Pelling, “Biocompatibility of Subcutaneously Implanted Plant-Derived Cellulose Biomaterials,” PLoS One, vol. 11, no. 6, p. e0157894, June 2016.

[16] R. J. Hickey, D. J. Modulevsky, C. M. Cuerrier, and A. E. Pelling, “Customizing the Shape and Microenvironment Biochemistry of Biocompatible Macroscopic Plant-Derived Cellulose Scaffolds,” ACS Biomater. Sci. Eng., p. acsbiomaterials.8b00178, March 2018.

[17] G. Fontana et al., “Biofunctionalized Plants as Diverse Biomaterials for Human Cell Culture,” Adv. Healthc. Mater., vol. 6, no. 8, p. 1601225, April 2017.

[18] J. R. Gershlak et al., “Crossing kingdoms: Using decellularized plants as perfusable tissue engineering scaffolds,” Biomaterials, vol. 125, pp. 13-22, May 2017.

[19] S. Torgbo and P. Sukyai, “Bacterial cellulose-based scaffold materials for bone tissue engineering,” Appl. Mater. Today, vol. 11, pp. 34-49, June 2018.

[20] J. Schindelin et al., “Fiji: an open-source platform for biological-image analysis,” Nat. Methods, vol. 9, no. 7, pp. 676-682, July 2012.

[21] M. Das Murtey and P. Ramasamy, “Sample Preparations for Scanning Electron Microscopy—Life Sciences,” in Modern Electron Microscopy in Physical and Life Sciences, InTech, 2016.

[22] W. N. Addison et al., “Extracellular matrix mineralization in murine MC3T3-E1 osteoblast cultures: An ultrastructural, compositional and comparative analysis with mouse bone,” Bone, vol. 71, pp. 244-256, February 2015.

[23] J. Lee, H. Jung, N. Park, S. H. Park, and J. H. Ju, “Induced Osteogenesis in Plants Decellularized Scaffolds,” Sci. Rep., vol. 9, no. 1, pp. 1-10, December 2019.

[24] J. Zhao et al., “Enhanced healing of rat calvarial defects with sulfated chitosan-coated calcium-deficient hydroxyapatite/bone morphogenetic protein 2 scaffolds.,” Tissue Eng. Part A, vol. 18, no. 1-2, pp. 185-97, January 2012.

[25] T. Zhang et al., “Effect of matrix stiffness on osteoblast functionalization,” Cell Prolif, vol. 50, no. 3, p. e12338, June 2017.

[26] H. Lv et al., “Mechanism of regulation of stem cell differentiation by matrix stiffness,” Stem Cell Research and Therapy, vol. 6, no. 1. BioMed Central Ltd., 27 May 2015.

[27] F. Han, C. Zhu, Q. Guo, H. Yang, and B. Li, “Cellular modulation by the elasticity of biomaterials,” Journal of Materials Chemistry B, vol. 4, no. 1. Royal Society of Chemistry, pp. 9-26, 17 Dec. 2016.

[28] J. Zhong, Y. Yang, L. Liao, and C. Zhang, “Matrix stiffness-regulated cellular functions under different dimensionalities,” Biomaterials Science, vol. 8, no. 10. Royal Society of Chemistry, pp. 2734-2755, 21 May 2020.

[29] X. Gao, M. Fraulob, and G. Haïat, “Biomechanical behaviours of the bone-implant interface: A review,” Journal of the Royal Society Interface, vol. 16, no. 156. Royal Society Publishing, 2019.

[30] S. P. Nukavarapu, J. W. Freeman, and C. T. Laurencin, Regenerative Engineering of Musculoskeletal Tissues and Interfaces. Elsevier Inc., 2015.

[31] J. Schindelin et al., “Fiji: an open-source platform for biological-image analysis,” Nat. Methods, vol. 9, no. 7, pp. 676-682, July 2012.

[32] M. Das Murtey and P. Ramasamy, “Sample Preparations for Scanning Electron Microscopy—Life Sciences,” in Modern Electron Microscopy in Physical and Life Sciences, InTech, 2016.

[33] P. P. Spicer, J. D. Kretlow, S. Young, J. A. Jansen, F. K. Kasper, and A. G. Mikos, “Evaluation of bone regeneration using the rat critical size calvarial defect,” Nat. Protoc., vol. 7, no. 10, pp. 1918-1929, September 2012.

[34] R. F. Heary, N. Parvathreddy, S. Sampath, and N. Agarwal, “Elastic modulus in the selection of interbody implants,” J. Spine Surg., vol. 3, no. 2, pp. 163-167, June 2017.

[35] W. N. Addison et al., “Extracellular matrix mineralization in murine MC3T3-E1 osteoblast cultures: An ultrastructural, compositional and comparative analysis with mouse bone,” Bone, vol. 71, pp. 244-256, February 2015.

[36] J. Zhao et al., “Enhanced Healing of Rat Calvarial Defects with Sulfated Chitosan-Coated Calcium-Deficient Hydroxyapatite/Bone Morphogenetic Protein 2 Scaffolds,” Tissue Eng. Part A, vol. 18, no. 1-2, pp. 185-197, January 2012.

[37] Rutkovskiy, A., Stensløkken, K. O., & Vaage, I. J. (2016). Osteoblast Differentiation at a Glance. Medical science monitor basic research, 22, 95-106. https://doi.org/10.12659/msmbr.901142.

[38] C. A. Gregory, W. G. Gunn, A. Peister, and D. J. Prockop, “An Alizarin red-based assay of mineralization by adherent cells in culture: Comparison with cetylpyridinium chloride extraction,” Anal. Biochem., vol. 329, no. 1, pp. 77-84, June 2004.

[39] P. Andrzejowski and P. V. Giannoudis, “The ‘diamond concept’ for long bone non-union management,” Journal of Orthopaedics and Traumatology, vol. 20, no. 1. Springer-Verlag Italia s.r.l., 1 Dec. 2019.

[40] A. Sakkas, F. Wilde, M. Heufelder, K. Winter, and A. Schramm, “Autogenous bone grafts in oral implantology—is it still a ‘gold standard’? A consecutive review of 279 patients with 456 clinical procedures,” Int. J. Implant Dent., vol. 3, no. 1, December 2017.

[41] S. N. Parikh, “Bone graft substitutes: past, present, future.,” J. Postgrad. Med., vol. 48, no. 2, pp. 142-8.

[42] W. Wang and K. W. K. Yeung, “Bone grafts and biomaterials substitutes for bone defect repair: A review,” Bioactive Materials, vol. 2, no. 4. KeAi Communications Co., pp. 224-247, 1 Dec. 2017.

[43] V. Campana et al., “Bone substitutes in orthopaedic surgery: from basic science to clinical practice,” J. Mater. Sci. Mater. Med., vol. 25, no. 10, pp. 2445-2461, September 2014.

[44] R. F. Fernandez, C. Bucchi, P. Navarro, V. Beltrán, and E. Borie, “Bone grafts utilized in dentistry: an analysis of patients' preferences,” BMC Med. Ethics, vol. 16, no. 1, p. 71, October 2015.

[45] X. Gao, M. Fraulob, and G. Haïat, “Biomechanical behaviours of the bone-implant interface: A review,” Journal of the Royal Society Interface, vol. 16, no. 156. Royal Society Publishing, 2019.

[46] S. P. Nukavarapu, J. W. Freeman, and C. T. Laurencin, Regenerative Engineering of Musculoskeletal Tissues and Interfaces. Elsevier Inc., 2015.

[47] K. C. Morrell, W. A. Hodge, D. E. Krebs, and R. W. Mann, “Corroboration of in vivo cartilage pressures with implications for synovial joint tribology and osteoarthritis causation,” Proc. Natl. Acad. Sci. U. S. A., vol. 102, no. 41, pp. 14819-14824, October 2005.

[48] L. F. Bonewald and M. L. Johnson, “Osteocytes, mechanosensing and Wnt signaling,” Bone, vol. 42, no. 4. NIH Public Access, pp. 606-615, April 2008.

[49] D. Zhang, S. Weinbaum, and S. C. Cowin, “Estimates of the peak pressures in bone pore water,” J. Biomech. Eng., vol. 120, no. 6, pp. 697-703, December 1998.

[50] M. Brunelli, C. Perrault, and D. Lacroix, “A Review of Bioreactors and Mechanical Stimuli,” Springer, Singapore, 2019, pp. 1-22.

[51] R. Pörtner, S. Nagel-Heyer, C. Goepfert, P. Adamietz, and N. M. Meenen, “Bioreactor design for tissue engineering,” Journal of Bioscience and Bioengineering, vol. 100, no. 3. Elsevier, pp. 235-245, 1 Sep. 2005.

[52] J. R. Henstock, M. Rotherham, J. B. Rose, and A. J. El Haj, “Cyclic hydrostatic pressure stimulates enhanced bone development in the foetal chick femur in vitro,” Bone, vol. 53, no. 2, pp. 468-477, April 2013.

[53] Y. Reinwald et al., “Evaluation of the growth environment of a hydrostatic force bioreactor for preconditioning of tissue-engineered constructs,” Tissue Eng.—Part C Methods, vol. 21, no. 1, pp. 1-14, January 2015.

[54] Y. Reinwald and A. J. El Haj, “Hydrostatic pressure in combination with topographical cues affects the fate of bone marrow-derived human mesenchymal stem cells for bone tissue regeneration,” J. Biomed. Mater. Res.—Part A, vol. 106, no. 3, pp. 629-640, Mar. 2018.

[55] Y. H. Zhao et al., “Hydrostatic pressure promotes the proliferation and osteogenic/chondrogenic differentiation of mesenchymal stem cells: The roles of RhoA and Racl,” Stem Cell Res., vol. 14, no. 3, pp. 283-296, May 2015.

[56] C. Liu, Y. Zhao, W. Y. Cheung, R. Gandhi, L. Wang, and L. You, “Effects of cyclic hydraulic pressure on osteocytes,” Bone, vol. 46, no. 5, pp. 1449-1456, May 2010.

[57] J. D. Gardinier, S. Majumdar, R. L. Duncan, and L. Wang, “Cyclic hydraulic pressure and fluid flow differentially modulate cytoskeleton re-organization in MC3T3 osteoblasts,” Cell. Mol. Bioeng., vol. 2, no. 1, pp. 133-143, Mar. 2009.

[58] E. Stavenschi, M. A. Corrigan, G. P. Johnson, M. Riffault, and D. A. Hoey, “Physiological cyclic hydrostatic pressure induces osteogenic lineage commitment of human bone marrow stem cells: A systematic study,” Stem Cell Res. Ther., vol. 9, no. 1, October 2018.

[59] D. J. Modulevsky, C. Lefebvre, K. Haase, Z. Al-Rekabi, and A. E. Pelling, “Apple derived cellulose scaffolds for 3D mammalian cell culture.,” PLoS One, vol. 9, no. 5, p. e97835, January 2014.

[60] D. J. Modulevsky, C. M. Cuerrier, and A. E. Pelling, “Biocompatibility of Subcutaneously Implanted Plant-Derived Cellulose Biomaterials.,” PLoS One, vol. 11, no. 6, p. e0157894, 2016.

[61] R. J. Hickey, D. J. Modulevsky, C. M. Cuerrier, and A. E. Pelling, “Customizing the Shape and Microenvironment Biochemistry of Biocompatible Macroscopic Plant-Derived Cellulose Scaffolds,” ACS Biomater. Sci. Eng., p. acsbiomaterials.8b00178, March 2018.

[62] J. Lee, H. Jung, N. Park, S. H. Park, and J. H. Ju, “Induced Osteogenesis in Plants Decellularized Scaffolds,” Sci. Rep., vol. 9, no. 1, pp. 1-10, December 2019.

[63] G. Fontana et al., “Biofunctionalized Plants as Diverse Biomaterials for Human Cell Culture,” Adv. Healthc. Mater., vol. 6, no. 8, p. 1601225, April 2017.

[64] L. D. Quarles, D. A. Yohay, L. W. Lever, R. Caton, and R. J. Wenstrup, “Distinct proliferative and differentiated stages of murine MC3T3-E1 cells in culture: An in vitro model of osteoblast development,” J. Bone Miner. Res., vol. 7, no. 6, pp. 683-692, June 1992.

[65] J. Schindelin et al., “Fiji: an open-source platform for biological-image analysis,” Nat. Methods, vol. 9, no. 7, pp. 676-682, July 2012.

[66] C. A. Gregory, W. G. Gunn, A. Peister, and D. J. Prockop, “An Alizarin red-based assay of mineralization by adherent cells in culture: Comparison with cetylpyridinium chloride extraction,” Anal. Biochem., vol. 329, no. 1, pp. 77-84, June 2004.

[67] J. D. Gardinier, V. Gangadharan, L. Wang, and R. L. Duncan, “Hydraulic pressure during fluid flow regulates purinergic signaling and cytoskeleton organization of osteoblasts,” Cell. Mol. Bioeng., vol. 7, no. 2, pp. 266-277, April 2014.

[68] D. Hong et al., “Morphological and proteomic analysis of early stage of osteoblast differentiation in osteoblastic progenitor cells,” Exp. Cell Res., vol. 316, no. 14, pp. 2291-2300, 2010.

[69] E. E. Golub and K. Boesze-Battaglia, “The role of alkaline phosphatase in mineralization,” Curr. Opin. Orthop., vol. 18, no. 5, pp. 444-448, September 2007.

All references cited here and elsewhere in the specification are herein incorporated by reference in their entireties.

What is claimed is:

1. A scaffold biomaterial comprising:
a decellularized plant or fungal tissue from which cellular materials and nucleic acids of the tissue are removed, the decellularized plant or fungal tissue comprising a 3-dimensional porous structure; and
a protein-based hydrogel, a polysaccharide-based hydrogel, or both.

2. The scaffold biomaterial of claim 1, wherein the protein-based hydrogel comprises collagen, osteonectin, osteopontin, bone sialoprotein, osteocalcin, fibronectin, laminin, a proteoglycan, bone morphogenetic protein, other matrix protein(s), or any combinations thereof; the polysaccharide-based hydrogel comprises agarose, alginate, hyaluronic acid, or another carbohydrate or a combination thereof; or both.

3. The scaffold biomaterial of claim 1 or 2, wherein the protein-based hydrogel comprises a collagen hydrogel.

4. The scaffold biomaterial of any one of claims 1-3, wherein the protein-based hydrogel comprises collagen I.

5. The scaffold biomaterial of any one of claims 1-4, wherein the decellularized plant or fungal tissue comprises a pore size of about 100 to about 200 μm, or of about 150 to about 200 μm.

6. The scaffold biomaterial of any one of claims 1-5, wherein the decellularized plant or fungal tissue comprises decellularized apple hypanthium tissue.

7. The scaffold biomaterial of any one of claims 1-6, wherein the scaffold biomaterial further comprises one or more bone-relevant cell types such as preosteoblasts, osteoblasts, osteoclasts, mesenchymal stem cells, differentiated bone and/or calvaria tissue cells, or any combinations thereof.

8. The scaffold biomaterial of any one of claims 1-7, having a Young's moduli between about 20 kPa to about 1 MPa.

9. The scaffold biomaterial of claim 7, wherein pore walls of the decellularized plant or fungal tissue are mineralized by the osteoblasts.

10. The scaffold biomaterial of any one of claims 1-9, wherein the decellularized plant or fungal tissue is at least partially coated or mineralized.

11. The scaffold biomaterial of claim 10, wherein the decellularized plant or fungal tissue is at least partially coated or mineralized with apatite, osteocalcium phosphate, a biocompatible ceramic, a biocompatible glass, a biocompatible metal nanoparticle, nanocrystalline cellulose, or any combinations thereof.

12. The scaffold biomaterial of claim 10 or 11, wherein the decellularized plant or fungal tissue is at least partially coated or mineralized with apatite.

13. The scaffold biomaterial of claim 12, wherein the apatite comprises hydroxyapatite.

14. A scaffold biomaterial comprising:
a decellularized plant or fungal tissue from which cellular materials and nucleic acids of the tissue are removed, the decellularized plant or fungal tissue comprising a 3-dimensional porous structure;
the decellularized plant or fungal tissue being at least partially coated or mineralized.

15. The scaffold biomaterial of claim 14, wherein the decellularized plant or fungal tissue is at least partially coated or mineralized with apatite, osteocalcium phosphate, a biocompatible ceramic, a biocompatible glass, a biocompatible metal nanoparticle, nanocrystalline cellulose, or any combinations thereof.

16. The scaffold biomaterial of claim 14 or 15, wherein the decellularized plant or fungal tissue is at least partially coated or mineralized with apatite.

17. The scaffold biomaterial of claim 16, wherein the apatite comprises hydroxyapatite.

18. The scaffold biomaterial of any one of claims 14-17, wherein the decellularized plant or fungal tissue comprises apple.

19. The scaffold biomaterial of any one of claims 14-18, wherein the decellularized plant or fungal tissue is at least partially coated or mineralized with apatite by alternating exposure to solutions of calcium chloride and disodium phosphate.

20. The scaffold biomaterial of any one of claims 14-19, wherein the scaffold biomaterial further comprises a protein-based hydrogel, a polysaccharide-based hydrogel, or both.

21. The scaffold biomaterial of claim 20, wherein the protein-based hydrogel comprises collagen, osteonectin, osteopontin, bone sialoprotein, osteocalcin, fibronectin, laminin, a proteoglycan, bone morphogenetic protein, other matrix protein(s), or any combinations thereof; the polysaccharide-based hydrogel comprises agarose, alginate, hyaluronic acid, or another carbohydrate or a combination thereof; or both.

22. The scaffold biomaterial of claim 20 or 21, wherein the protein-based hydrogel comprises a collagen hydrogel.

23. The scaffold biomaterial of any one of claims 20-22, wherein the protein-based hydrogel comprises collagen I.

24. The scaffold biomaterial of any one of claims 1-23, wherein the decellularized plant or fungal tissue is cellulose-based, chitin-based, chitosan-based, lignin-based, hemicellulose-based, or pectin-based, or any combination thereof.

25. The scaffold biomaterial of any one of claims 1-24, wherein the plant or fungal tissue comprises a tissue from apple hypanthium (Malus pumila) tissue, a fern (Monilophytes) tissue, a turnip (Brassica rapa) root tissue, a gingko branch tissue, a horsetail (equisetum) tissue, a hermocallis hybrid leaf tissue, a kale (Brassica oleracea) stem tissue, a conifers Douglas Fir (Pseudotsuga menziesii) tissue, a cactus fruit (pitaya) flesh tissue, a Maculata Vinca tissue, an Aquatic Lotus (Nelumbo nucifera) tissue, a Tulip (Tulipa gesneriana) petal tissue, a Plantain (Musa paradisiaca) tissue, a broccoli (Brassica oleracea) stem tissue, a maple leaf (Acer psuedoplatanus) stem tissue, a beet (Beta vulgaris) primary root tissue, a green onion (Allium cepa) tissue, a orchid (Orchidaceae) tissue, turnip (Brassica rapa) stem tissue, a leek (Allium ampeloprasum) tissue, a maple (Acer) tree branch tissue, a celery (Apium graveolens) tissue, a green onion (Allium cepa) stem tissue, a pine tissue, an aloe vera tissue, a watermelon (Citrullus lanatus var. lanatus) tissue, a Creeping Jenny (Lysimachia nummularia) tissue, a cactae tissue, a Lychnis Alpina tissue, a rhubarb (Rheum rhabarbarum) tissue, a pumpkin flesh (Cucurbita pepo) tissue, a Dracena (Asparagaceae) stem tissue, a Spiderwort (Tradescantia virginiana) stem tissue, an Asparagus (Asparagus officinalis) stem tissue, a mushroom (Fungi) tissue, a fennel (Foeniculum vulgare) tissue, a rose (Rosa) tissue, a carrot (Daucus carota) tissue, or a pear (Pomaceous) tissue, or a genetically altered tissue produced via direct genome modification or through selective breeding, or any combinations thereof.

26. The scaffold biomaterial of any one of claims 1-25, further comprising living cells, in particular non-native cells, on and/or within the decellularized plant or fungal tissue.

27. The scaffold biomaterial of claim 26, wherein the living cells are animal cells.

28. The scaffold biomaterial of claim 27, wherein the living cells are mammalian cells.

29. The scaffold biomaterial of claim 28, wherein the living cells are human cells.

30. The scaffold biomaterial of any one of claims 1-29, comprising two or more subunits which are glued, cross-linked, or interlocked together.

31. The scaffold biomaterial of any one of claims 1-30, wherein the decellularized plant or fungal tissue comprises two or more different decellularized plant or fungal tissues derived from different tissues or different sources.

32. The scaffold biomaterial of claim 31, wherein the two or more different decellularized plant or fungal tissues are glued, cross-linked, or interlocked together.

33. The scaffold biomaterial of any one of claims 1-32, for use in bone tissue engineering.

34. A bone graft comprising the scaffold biomaterial of any one of claims 1-33.

35. Use of the scaffold biomaterial of any one of claims 1-32 for bone tissue engineering, for bone grafting, for repair or regeneration of bone, for osteoblast differentiation, or any combination thereof.

36. Use of the scaffold biomaterial of any one of claims 1-32 for any one or more of: craniofacial reconstructive surgery; dental and/or maxillofacial reconstructive surgery; major bone defect and/or trauma reconstruction; bone filler applications; implant stabilization; and/or drug delivery; or any combinations thereof.

37. Use of the scaffold biomaterial of any one of claims 1-32 in a dental bone filler application.

38. Use of the scaffold biomaterial of any one of claims 1-32 as stress shielding reducers for large implants.

39. Use of the scaffold biomaterial of any one of claims 1-32 for promoting active osteogenesis; for implanting to repair critical and/or non-critical size defects; to provide mechanical support during bone repair; to substitute in loss or injury of long bones, calvarial bones, maxillofacial bones, dental, and/or jaw bones; for orthodontal and/or peri dental grafts, such as alveolar ridge augmentation, tooth loss, tooth implants and/or reconstructive surgery; for grafting at specific site(s) to augment bone volume due to loss from osteoporosis, bone loss due to age, previous implant, and/or injuries; or to improve bone-implant tissue integration; or any combinations thereof.

40. A method for engineering bone tissue; for bone grafting; for repair or regeneration of bone; for craniofacial reconstructive surgery; for dental and/or maxillofacial reconstructive surgery; for major bone defect and/or trauma reconstruction; for dental or other bone filler application; for implant stabilization; for stress shielding of a large implant; for promoting active osteogenesis; for repairing critical and/or non-critical size defects; for provide mechanical support during bone repair; for substituting for loss or injury of long bones, calvarial bones, maxillofacial bones, dental, and/or jaw bones; for orthodontal and/or peri dental grafting such as alveolar ridge augmentation, tooth loss, tooth implants and/or reconstructive surgery; for grafting at a specific site to augment bone volume due to loss from osteoporosis, bone loss due to age, previous implant, and/or injuries; for improving bone-implant tissue integration; or for drug delivery; or for any combinations thereof; said method comprising:
providing a scaffold biomaterial as defined in any one of claims 1-32; and
implanting the scaffold biomaterial into a subject in need thereof at a site or region in need thereof.

41. A method for producing a scaffold biomaterial, said method comprising:
providing a decellularized plant or fungal tissue from which cellular materials and nucleic acids of the tissue are removed, the decellularized plant or fungal tissue comprising a 3-dimensional porous structure; and
introducing a protein-based hydrogel, a polysaccharide-based hydrogel, or both into the decellularized plant or fungal tissue.

42. The method of claim 41, wherein the protein-based hydrogel comprises collagen, osteonectin, osteopontin, bone sialoprotein, osteocalcin, fibronectin, laminin, a proteoglycan, bone morphogenetic protein, other matrix protein(s), or any combinations thereof; the polysaccharide-based hydrogel comprises agarose, alginate, hyaluronic acid, or another carbohydrate or a combination thereof; or both.

43. The method of claim 41 or 42, wherein the protein-based hydrogel comprises a collagen hydrogel.

44. The method of any one of claims 41-43, wherein the protein-based hydrogel comprises collagen I.

45. A method for producing a scaffold biomaterial, said method comprising:
providing a decellularized plant or fungal tissue from which cellular materials and nucleic acids of the tissue are removed, the decellularized plant or fungal tissue comprising a 3-dimensional porous structure; and
at least partially coating or mineralizing the decellularized plant or fungal tissue.

46. The method of claim 45, wherein the decellularized plant or fungal tissue is at least partially coated or mineralized with apatite, osteocalcium phosphate, a biocompatible ceramic, a biocompatible glass, a biocompatible metal nanoparticle, nanocrystalline cellulose, or any combinations thereof.

47. The method of claim 45 or 46, wherein the decellularized plant or fungal tissue is at least partially coated or mineralized with apatite.

48. The method of claim 46 or 47, wherein the apatite comprises hydroxyapatite.

49. The method of any one of claims 45-48, wherein the step of coating or mineralizing the decellularized plant or fungal tissue comprises subjecting the decellularized plant or fungal tissue to alternating exposures to solutions of calcium chloride and disodium phosphate.

50. The method of any one of claims 45-49, wherein the method further comprises introducing a protein-based hydrogel, a polysaccharide-based hydrogel, or both, to the scaffold biomaterial.

51. The method of claim 50, wherein the protein-based hydrogel comprises collagen, osteonectin, osteopontin, bone sialoprotein, osteocalcin, fibronectin, laminin, a proteoglycan, bone morphogenetic protein, other matrix protein(s), or any combinations thereof; the polysaccharide-based hydrogel comprises agarose, alginate, hyaluronic acid, or another carbohydrate or a combination thereof; or both.

52. The method of claim 50 or 51, wherein the protein-based hydrogel comprises a collagen hydrogel.

53. The method of any one of claims 50-52, wherein the protein-based hydrogel comprises collagen I.

54. The method of any one of claims 41-53, further comprising a step of introducing living cells, in particular non-native cells, on and/or within the decellularized plant or fungal tissue.

55. The method of claim 54, wherein the living cells are animal cells.

56. The method of claim 55, wherein the living cells are mammalian cells.

57. The method of claim 56, wherein the living cells are human cells.

58. The method of claim 57, wherein the cells are preosteoblasts, osteoblasts, osteoclasts, mesenchymal stem cells, differentiated bone and/or calvaria tissue cells, or any combinations thereof.

59. A kit comprising any one or more of:
a decellularized plant or fungal tissue from which cellular materials and nucleic acids of the tissue are removed, the decellularized plant or fungal tissue comprising a 3-dimensional porous structure;
a protein-based hydrogel;
a polysaccharide-based hydrogel;
apatite;
calcium chloride;
disodium phosphate;
osteocalcium phosphate;
a biocompatible ceramic;
a biocompatible glass;
a biocompatible metal nanoparticle;
nanocrystalline cellulose;
mammalian cells, such as one or more bone-relevant cell types such as preosteoblasts, osteoblasts, osteoclasts, mesenchymal stem cells, differentiated bone and/or calvaria tissue cells, or any combinations thereof;
plant or fungal tissue, decellularization reagents, or both;
a buffer; and/or
instructions for performing a method as defined in any one of claims 40-58.

60. The kit of claim 59, wherein the protein-based hydrogel comprises collagen, osteonectin, osteopontin, bone sialoprotein, osteocalcin, fibronectin, laminin, a proteoglycan, bone morphogenetic protein, other matrix protein(s), or any combinations thereof; the polysaccharide-based hydrogel comprises agarose, alginate, hyaluronic acid, or another carbohydrate or a combination thereof; or both.

61. The kit of claim 59 or 60, wherein the protein-based hydrogel comprises a collagen hydrogel.

62. The kit of any one of claims 59-61, wherein the protein-based hydrogel comprises collagen I.

63. The kit of any one of claims 59-62, wherein the apatite comprises hydroxyapatite.

64. A method for differentiating cartilage or bone precursor cells to become cartilage or bone tissue cells, said method comprising:
culturing the cartilage or bone precursor cells on a scaffold biomaterial as defined in any one of claims 1-33 in a differentiation media;
wherein the culturing includes exposing the cultured cells to an increased atmospheric pressure above ambient pressure at least once.

65. Use of a scaffold biomaterial according to any one of claims 1-33 for differentiating cartilage or bone precursor cells to become cartilage or bone tissue cells, wherein the scaffold biomaterial is for use in culturing the cartilage or bone precursor cells in a differentiation media, the culturing including exposing the cells to an increased atmospheric pressure above ambient pressure at least once.

66. A method for differentiating cartilage or bone precursor cells to become cartilage or bone tissue cells, said method comprising:
culturing the cartilage or bone precursor cells on a scaffold biomaterial as defined in any one of claims 1-33 in a differentiation media;
wherein the culturing includes at least one treatment period during which the cultured cells are exposed to an increased atmospheric pressure above ambient pressure for at least part of the treatment period, wherein the treatment period is at least about 10 minutes in duration and is performed at least once per week;
thereby differentiating the cartilage or bone precursor cells into cartilage or bone tissue cells.

67. The method of claim 66, wherein the cultured cells are returned to a low or ambient pressure condition after each exposure to the increased atmospheric pressure.

68. The method of claim 66 or 67, wherein the treatment period comprises alternating the cultured cells between a low or ambient pressure condition, and an increased atmospheric pressure condition.

69. The method of any one of claims 66-68, wherein the treatment period comprises oscillating atmospheric pressure to which the cells are exposed between a low or ambient pressure and an increased atmospheric pressure.

70. The method of any one of claims 66-68, wherein the treatment period comprises oscillating atmospheric pressure to which the cells are exposed between a low or ambient pressure and an increased atmospheric pressure at a frequency of about 1-10 Hz.

71. The method of any one of claims 66-70, wherein the treatment period comprises oscillating atmospheric pressure to which the cells are exposed between a low or ambient pressure and an increased atmospheric pressure, wherein the low or ambient pressure is ambient pressure, such as about 101 kPa, and the increased atmospheric pressure is about +280 kPa above ambient pressure, such as about 381 kPa, and optionally wherein the oscillating is at a frequency of about 1-10 Hz.

72. The method of claim 66 or 67, wherein the treatment period comprises exposing the cultured cells to increased atmospheric pressure for a sustained duration.

73. The method of any one of claim 66, 67, or 72, wherein the treatment period comprises exposing the cultured cells to a substantially constant increased atmospheric pressure for a sustained duration.

74. The method of any one of claims 66-73, wherein the treatment period is about 1 hour in duration, or longer.

75. The method of any one of claims 66-74, wherein the treatment period is performed once daily, or more than once daily.

76. The method of any one of claims 66-75, wherein the culturing is performed for at least about 1 week.

77. The method of any one of claims 66-76, wherein the culturing is performed for about 2 weeks, or longer.

78. The method of any one of claims 66-77, wherein the increased atmospheric pressure is applied as hydrostatic pressure.

79. The method of any one of claims 66-78, wherein the increased atmospheric pressure is applied by modulating the pressure of a gas phase above the cultured cells.

80. The method of any one of claims 66-79, wherein the increased atmospheric pressure is about +280 kPa above ambient pressure, such as about 381 kPa.
